(19) United States
(12) Patent Application Publication
(10) Pub. No.: US 2002/0022257 A1
(43) Pub. Date: Feb. 21, 2002

(54) Three-dimensional structure and crystallization method of ribosome recycling factor (RRF)

(76) Inventors: Se Won Suh, Seoul (KR); Kyeong Kyu Kim, Seoul (KR); Kyeong Sik Min, Kyeongsangnam-do (KR); Sung Chul Ha, Kyeongsangnam-do (KR); Soo Hyun Eom, Seoul (KR); Wook Hyun Kim, Seoul (KR); Jung Min Yun, Seoul (KR)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/731,487

(22) Filed: Dec. 7, 2000

(30) Foreign Application Priority Data
Jul. 1, 2000 (KR) 2000.37527

(51) Int. Cl. C12N 9/22; G06F 19/00

(52) U.S. Cl. 435/199; 702/19

(57) ABSTRACT

A crystal of E. coli RRF complexed with a detergent is disclosed as well as the three-dimensional structure of the complex as analyzed by X-ray diffraction. The crystallization method and a method for identifying an inhibitor of RRF based on the identified three-dimensional structure is also disclosed. The RRF crystal was obtained in the presence of a detergent, and the three-dimensional structure of RRF was analyzed from this crystal. The active site forming a hydrophobic cleft and the binding site of RRF to the A-site of the ribosome was identified in the three-dimensional structure of RRF. The three-dimensional structure of RRF, and particularly, the active site and binding site can aid in the identification or modeling of an inhibitor for RRF. Because RRF is essential for viability in prokaryotes, but is not essential in eukaryotes the inhibitor can be used as an antibiotic.

### FIELD OF THE INVENTION

The present invention relates to a crystal of ribosome recycling factor (RRF) and a method of crystallization. More particularly, the present invention relates to an RRF crystal formed by binding with a detergent and to the method of crystallization using the detergent. Further, the present invention relates to a protein complex comprising RRF or an RRF homolog and a molecule or material binding to the hydrophobic cleft formed between the two domains of RRF. The present invention further relates to a polypeptide comprising the structure of one domain of RRF which binds to the ribosome. The present invention further relates to a method for screening an RRF inhibitor using the three-dimensional structure of RRF. The present invention further relates to an RRF inhibitor comprising alkyl-β-D-maltopyranoside or its derivatives.

### BACKGROUND OF THE INVENTION

After the discovery and use of penicillin as an antibiotic, many antibiotic materials were developed to control pathogens. However, the appearance of new pathogens and antibiotic-resistant pathogens has continuously required the development of new antibiotics. Antibiotics for the purpose of suppressing the metabolism of bacteria such as the synthesis of the cell wall or protein biosynthesis have been the main type developed from natural compounds or their derivatives.

Recently, advances in protein crystallography and computational chemistry have introduced a new method of structure-based drug design into the field of drug development. The new method typically involves finding inhibitors by fitting inhibitor candidates to the active site of target proteins based on the high resolution crystal structure and computing the binding intensities or affinities in the complexes. This approach is useful for developing new antibiotics.

Protein biosynthesis consists of three steps: initiation, elongation and termination. In the termination step, the protein-releasing factor (RF1 and RF2 in prokaryotes and eRF1 in eukaryotes) helps to release the synthesized protein at the stop codon, which is then released from the A-site of the ribosome by releasing factor 3 (RF3) (Freistroffer et al., 1997). At this point, other factors remaining in the termination complex must be recycled so that the protein biosynthesis can proceed continuously (Kaji et al. 1998). Ribosome recycling factor (RRF) plays a necessary role in termination by catalyzing the hydrolysis of GTP and the release of the mRNA and tRNA from the ribosome. The process is aided by elongation factor G (EF-G) or release factor 3 (RF3) (Pavlov et al., 1997a; Kaji et al., 1998; Grentzmann et al., 1998; FIG. 1). RRF has been found to directly dissociate peptidyl-tRNA as well as deacylated tRNA from the ribosome (Heurgue-Hamard et al., 1998; Grentzmann et al., 1998). RRF has also been found to play a role in the prevention of errors generated in the elongation step of protein synthesis (Janosi et al., 1996). The importance of recycling the ribosome in the process of protein synthesis is evidenced by the fact that the growth of bacteria is suppressed when the activity of RRF is inhibited (Janosi et al., 1998). The absence of RRF caused cell death in Escherichia coli (Janosi et al., 1994), which may be due to increased translation errors (Janosi et al., 1996) and unscheduled reinitiation (Ryoji et al., 1981; Janosi et al., 1998).

The mechanism for the prevention of errors in the translation process or in the process of recycling of the ribosome is still unknown. RRF has been found to bind to the A-site of the ribosome and its binding is competitive with the binding of releasing factor 1 (RF1) (Pavlov et al., 1997b). Therefore, it is predicted, based on the binding affinity to the ribosome, that RRF will have structural similarity to other translation factors which bind to the A-site of the ribosome. Therefore, RRF is thought to also mimic the tRNA-like structure proposed in other translation factors (Nakamura et al., 1996; Brock et al., 1998). According to one model for recycling of the ribosome, RRF moves to the P-site after binding to the A-site of the ribosome (Nakamura et al., 1996) and deacylated tRNA is moved to the E-site (Janosi et al., 1996; Pavolv et al., 1997a). At this point, the complex is dissociated into mRNA, tRNA and the ribosome (Kaji et al., 1998) or it may dissociate into two units: 1) the tRNA and 2) the ribosome bound to the mRNA (Pavolv et al., 1997a). A mechanism proposed by more recent research differs from this in that RRF, EF-G and GTP only catalyze the dissociation of the 50S subunit from the termination complex, and tRNA is removed from the 30S:mRNA:tRNA complex by an initiation factor, IF3(Karimi et al., 1999).

Genes encoding RRFs are widely distributed in prokaryotes and are found to have very high homologies (Janosi et al., 1996). As a result of sequence analysis, it was found that the genes encoding RRFs also exist in eukaryotes. However, in eukaryotes they are mostly present in organelles (Janosi et al., 1996). The RRF homolog in yeast participates in protein synthesis in mitochondria (Janai et al., 1998), but is not essential for cell growth (Kaji et al., 1998). An RRF homolog located in the chloroplast of spinach exerts an inhibitory action on a bacterial temperature-sensitive mutant (Rolland et al., 1999). Therefore, it may be concluded that eukaryotic RRF is not important for cell viability and that the inhibition of the RRFs located in the organelles does not influence the synthesis of cytoplasmic proteins. This fact is also evidenced by the fact that RRF does not exist in Methanococcus jannashchii, an ancient microbe having a translation system similar to a eukaryote. Because of this, RRF is thought to be an excellent target for developing new antibacterial drugs (Kaji et al., 1998), since it is essential for prokaryotic cell viability, but not essential in eukaryotes. Although there is a chance that such an inhibitor may act on the mitochondria of eukaryotes, it can still be utilized in view of the fact that widely used antibiotics such as tetracycline and erythromycin also target protein synthesis in mitochondria. Further, from the results of recent studies on the RRF of P. aeruginosa as well as E. coli, it has been found that RRF is exchangeable between the two microbes. This fact suggests that an inhibitor with broad antibiotic specificity for prokaryotes can be developed from studies on E. coli RRF.

### SUMMARY OF THE INVENTION

It is, therefore, an object of the present invention to provide an RRF crystal and its crystallization method. The RRF crystal is the starting point for analyzing the three-dimensional structure of RRF, which is key to the design, screening and identification of RRF inhibitors.

It is another object of the present invention to provide a method for identifying RRF inhibitors by means of the three-dimensional structure of RRF. By the present invention, RRF inhibitor candidates can be screened easily, and RRF inhibitors with optimum activity can be designed and identified.

It is a further object of the present invention to provide RRF inhibitors including maltopyranosides and their derivatives, which are identified as inhibiting RRF.

It is still a further object of the present invention to provide a polypeptide having a structure comprising part or all of the coiled coil domain of RRF. Such a polypeptide may inhibit the activity of RRF by competitively binding to the RRF binding site of the ribosome, thereby preventing RRF from binding to the ribosome.

In accordance with one embodiment of the present invention, there is provided a crystal and a method for crystallization of RRF or an RRF homolog. The RRF crystal or an RRF homolog according to the present invention can be obtained by crystallizing it with a material or compound or molecule which binds to a specific site of the RRF. The RRF crystal according to the present invention includes RRF and the material which binds to the specific site of the RRF. The specific site of RRF has been found to be a hydrophobic cleft formed between two domains in the three-dimensional structure of RRF identified in the present invention. In the three-dimensional structure of RRF according to the present invention, the two domains are formed with an L-shape and the hydrophobic cleft is formed between the two domains. One domain of RRF can bind to the ribosome and the activity of RRF may be exerted within the hydrophobic cleft.

In accordance with a further embodiment of the present invention, there is provided a method for identifying RRF inhibitors based on the three-dimensional structure of RRF. The inhibitors may include maltopyranosides or their derivatives identified as having RRF inhibition according to the present invention.

In accordance with a further embodiment of the present invention, there is provided a polypeptide comprising a part or all of the coiled coil domain of RRF. The polypeptide can inhibit the activity of RRF by competitively binding to the RRF binding site of the ribosome, thereby preventing RRF from binding to the ribosome.

### BRIEF DESCRIPTION OF THE DRAWINGS

The above and other objects and features of the present invention will become apparent from the following description of preferred embodiments given in conjunction with the accompanying drawings, in which:

FIG. 1 is a ribbon diagram of the three-dimensional structure of RRF analyzed with the RRF crystal according to the present invention.

FIG. 2 provides a stick model showing a detergent molecule located in a cleft of RRF drawn from a surface model of the three-dimensional structure of RRF analyzed with the RRF crystal according to the present invention.

FIG. 3 shows the multiple sequence alignment of RRFs derived from prokaryotes. The E. coli sequence is SEQ ID NO:1, the H. influenza sequence is SEQ ID NO:2, the P. aeruginosa sequence is SEQ ID NO:3, and the B. subtilis sequence is SEQ ID NO:4.

FIG. 4 shows relative movement between the two domains of the RRF in the three-dimensional structure of RRF analyzed with the RRF crystal according to the present invention.

FIG. 5 provides the three-dimensional structural surface models of RRF with molecules binding to the A-site of the ribosome. The molecules shown are tRNA and EF-G and also shown is the structural similarities therebetween.

### DETAILED DESCRIPTION OF THE PREFERRED EMBODIMENTS

The invention will be illustrated by the following preferred embodiments with reference to the accompanying drawings.

Abbreviations and Definitions

1) Abbreviations
RRF: ribosome recycling factor
Amino acids

| Amino acids | One Letter Symbol | Three Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

2) Definitions

The definitions used in this specification are as follows:

A Crystal refers to a protein of crystalline form. A crystal is to be piled up with several unit cells.

The word Complex refers to the form of protein when the protein is bound with one or more materials, molecules or compounds.

A Unit cell is the simplest and smallest volume of unit in a crystal. It is defined with a, b, c, alpha, beta and gamma (Blundell et al., 1976).

A Space group is an arrangement of symmetric elements in a crystal. In the definition of space group, English capital letters refer to the lattice type and other numbers refer to symmetric operators.

An Asymmetric Unit refers to the largest unit that does not have a symmetric element in the unit cell, but is superimposed with the same shape by the symmetric operation of a space group.

Multiple isomorphous replacement is a method for obtaining information about the phase for three-dimensional structures by using heavy metal derivatives made from infiltrating the heavy metal solution into a crystal (Blundell et al., 1976).

A Molecular replacement is a method for obtaining a preliminary structure from a crystal of unknown structure by changing the locations of molecules having known structural coordinates.

The RRF homologs referred to in this application are meant to have at least 30% amino acid similarity to RRF and to include RRF mutants.

Crystallization of RRF and Structure of the Crystal

One object of the present invention is to obtain an RRF crystal from a solution containing a precipitant such as polyethylene glycol with a detergent having a hydrophobic part such as decyl-β-D-maltopyranoside. It is hypothesized that the RRF crystal may not be obtained free of the detergent because it has a hydrophobic part. As described below, the three-dimensional structure of RRF consists of two domains, which move relative to each other, and thus it is difficult to crystallize RRF without a means for fixing the two domains. Prior to this invention, other research groups had tried to crystallize the isolated RRF from E. coli with no success. Various methods were used to crystallize the isolated RRF from E. coli in solutions with various concentrations of the protein under various crystallization conditions with no success. The failure was likely caused due to the fact that the crystallization was not performed with a detergent having a hydrophobic part. It is likely that the detergent is necessary because it binds to the cleft formed between the two domains of RRF, thereby eliminating the relative movement of the two domains. Therefore, in order to crystallize RRF or an RRF mutant (or RRF homologue) isolated from E. coli or other bacteria, it is likely to be necessary to use a material which binds to the hydrophobic cleft of RRF as described below, preferably a detergent having a hydrophobic part.

Structure of RRF Crystal

The RRF crystal obtained by the method described above includes a detergent having a hydrophobic part, decyl-β-D-maltopyranoside. The RRF crystal of the present invention may be analyzed by X-ray diffraction to identify the three-dimensional structure of RRF. The X-ray diffraction data suggests that the RRF crystal of the present invention has a long stick shape with a unit cell of a=b=48.06 Å and c=142.27 Å, and a space group of P3₁21.

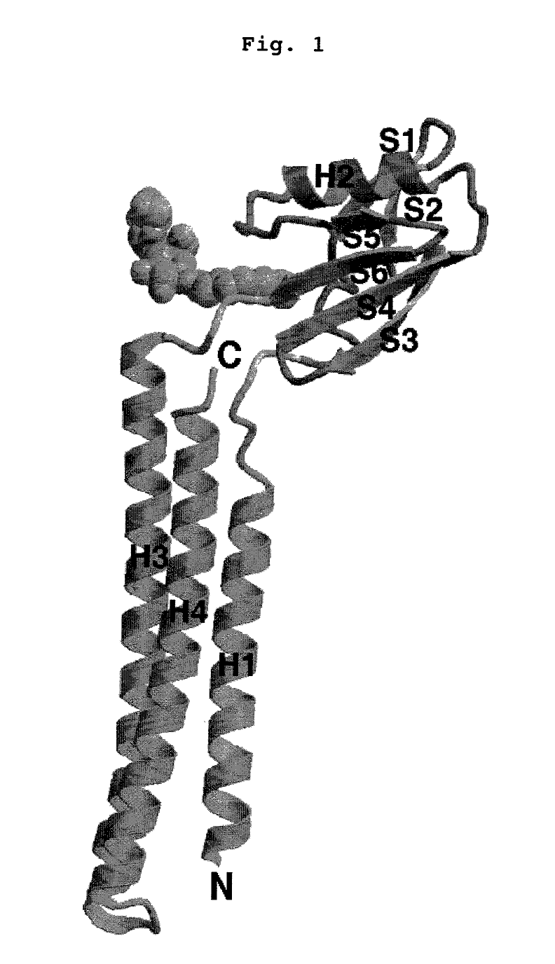

The three-dimensional structure of RRF is depicted in FIG. 1, which provides a ribbon diagram of the three-dimensional structure of RRF analyzed using the RRF crystal of the present invention. In FIG. 1, the two domains are linked with linkers, the arrows indicate strands and the cylinders indicate helices. The ends of amino acid and carboxylic acid are indicated respectively, and the detergent is depicted using a space filling model.

As shown in table 1, the three-dimensional structure of RRF was determined with 0.23 nm resolution to form a three-dimensional model for 1437 of the protein atoms, 230 of the water molecules and 16 atoms of decyl-β-D-maltopyronoside. The three-dimensional coordinate of each atom in the three-dimensional structure of RRF is shown in table 2. As shown in FIG. 1, RRF is an open L-shaped molecule comprising two domains. The N-terminal helix H1 of the first domain is linked with the second domain. Two antiparallel C-terminal α-helices H3 and H4 connected by a short U-turn are extended from the second domain and folded back onto the N-terminal helix H1. Thus, the first domain (coil domain) consists of a three-stranded antiparallel coiled-coil about 65 Å long, in which the seven-turn helix H1 packs into the parallel eleven-turn helix H3 and the antiparallel ten-turn helix H4. The two-turn overhang helices on the C-terminal end of H3 and the N-terminal end of H4 near the U-turn form a short two-stranded coiled-coil. The second domain (α/β domain) with approximate dimensions of 23×26×36 Å has an α/β topology containing a short β-hairpin (S1 and S2) and a single a-helix (H2) packed against one face of an antiparallel four-stranded β-sheet (S3-S6). Two domains are connected by two hinge residues (Arg31 and Pro103) with an approximate angle of 1100 between the lines along helix H3 and strand S6.

TABLE 1
X-ray diffraction data for RRF

| Data collection | |
|---|---|
| Resolution (Å) | 20.0-2.3 (2.38-2.30) |
| R_{merge} (%) | 4.8 (23.0) |
| Completeness (%) | 96.2 (97.7) |
| No. of unique reflections | 8,691 (858) |
| Redundancy | 3.34 |
| Refinement | |
| Resolution (Å) | 20.0-0.3 |
| R_{factor}/R_{free} (%) | 22.8/29.7 |
| No. of reflections (2 α cut off) | 8,391 |
| No. of solvent atoms | 230 |
| No. of protein atoms | 1437 |
| No. of Heteroatoms | 16 |
| Mean B factor (Å²) | 53.9 |
| R.m.s.d. in binding length (Å) | 0.010 |
| R.m.s.d in binding angle (°) | 1.63 |

The terms in the table are as follows:

Resolution: the distance between two plates when the diffraction conditions of Bragg are satisfied.

R_{merge}: the degree of accordance between measurements of diffraction intensities when they are measured repeatedly.

R_{factor}: the difference between the diffraction intensities obtained by experiment and calculated in the model.

R_{free}: R_{factor} calculated by using diffraction points excluded during the optimum process in order to measure the usefulness of the obtained model.

Hydrophobic Cleft and Movement of Domains

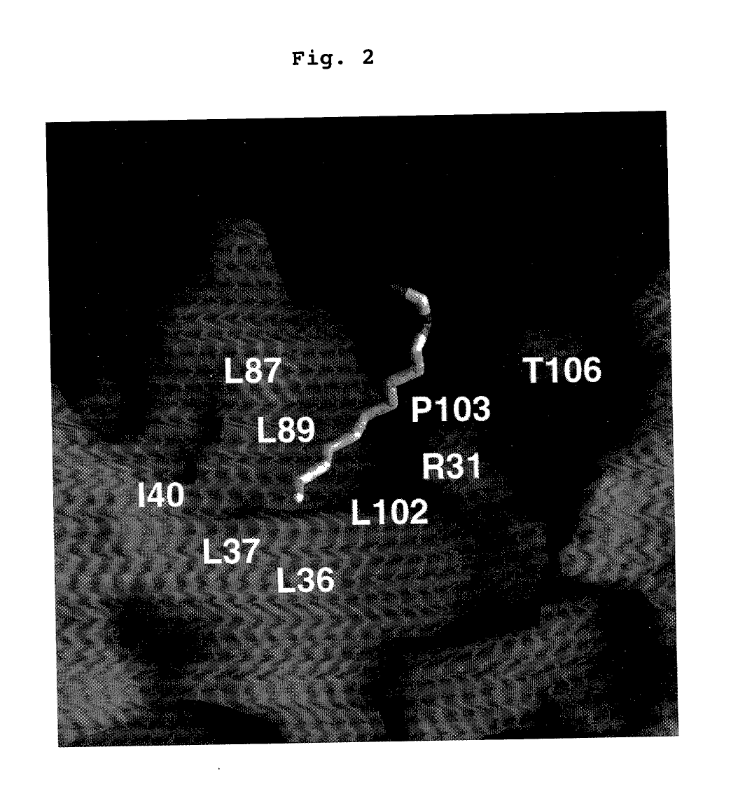

FIG. 2 provides a stick model showing a detergent located in a cleft of RRF drawn by a surface model of the RRF crystal. Amino acid residues are depicted which are involved in hydrophobic bonding with their names and location numbers (Thr-106, Arg-31, Pro-103, Leu-36, Leu-37, Ile-40, Leu-87, Leu-89, Leu-102) in the drawing. The head part of the detergent is not depicted, and is not involved in the hydrophobic bonding.

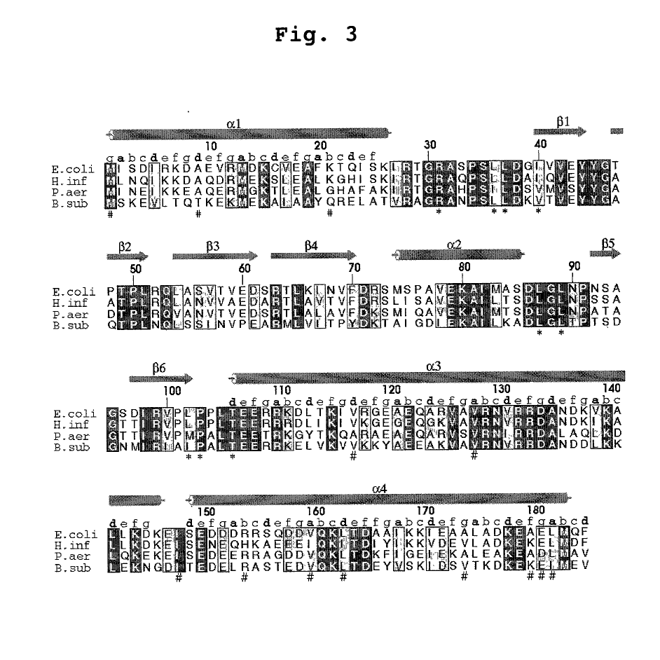

FIG. 3 shows multiple sequence alignments of RRFs derived from prokaryotes. The sequences are from the following prokaryotes: E. coli, Haemophilus influenzae (H. inf), Psuedomonas aeuruginosa (P. aeu) and Bacillus subtillis (B. sub). The secondary structure of the E. coli RRF is indicated by an arrow for a β-strand and by a cylinder for an α-helix. The amino acid residues that make contact with the alkyl chain of the detergent are marked with asterisks (*). The mutated residues identified in temperature-sensitive mutants of RRF (Janosi et al., 1998) are indicated by # marks. The heptad pattern (abc defg) of each residue in the coiled-coil is also shown. In the four sequences, identical residues are boxed in black and homologous residues are boxed in gray.

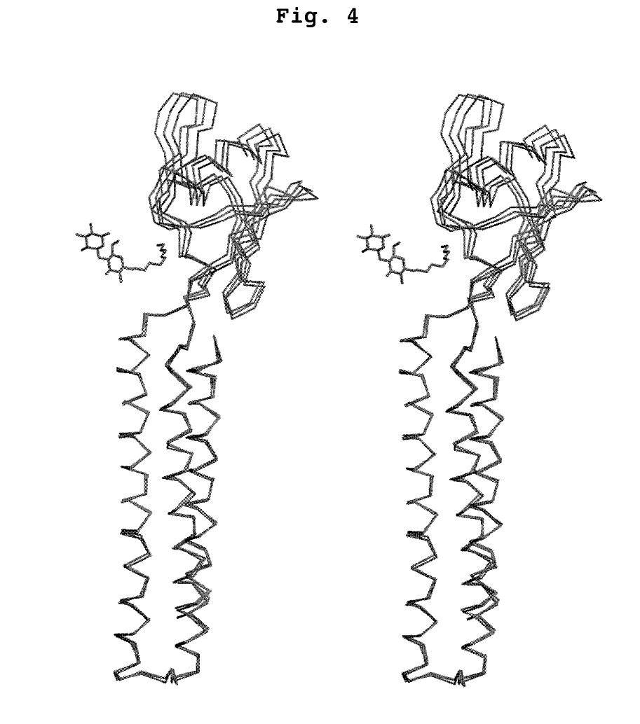

As shown in FIG. 2, the alkyl chain of the detergent, n-decyl-β-D-pyranomaltoside is located in the hydrophobic cleft formed at the joint between the two domains. Several hydrophobic amino acid residues form the hydrophobic cleft and interact hydrophobically with the alkyl chain of the detergent. As shown in FIG. 3, from the comparisons of the protein sequences of several RRFs it is clear that the amino acid residues involved in binding the detergent are very well conserved. Therefore, these amino acid residues are likely involved in binding other proteins or hydrophobic materials, and this binding is likely to be coupled with the activity of RRF. The contact of detergent in the hydrophobic cleft will decrease the relative movement between the two domains, which are linked through the cleft. In actuality, the relative positions of the two domains are not the same in different crystals, because they are movable. This is shown in FIG. 4.

In FIG. 4, the relative movement between the two domains of the RRF in the three-dimensional structure of RRF are analyzed using the RRF crystal according to the present invention. When superimposing the lower domains in the structures of RRFs obtained from the three different crystals of RRFs, the movement of upper domains is observed. The three different crystals of RRFs are named PT1 (left side), Native1 (center) and Native2 (right side). The detergent of Native1 is depicted in FIG. 4. It is believed that the movement would be more pronounced without the detergent. The relative movement of the two domains is a troublesome factor in crystallization of RRF itself, as described above.

It is predicted that RRF is likely to bind to other molecules or their parts at the hydrophobic cleft formed between the movable domains. In other words, the hydrophobic cleft may act as a binding site for other molecules or their parts. This means that it is likely that the hydrophobic cleft influences the function of RRF in protein synthesis.

The relative movement between the two domains of RRF and the hydrophobic cleft formed by the two domains of RRF are common phenomena discovered in molecules which bind to the A-site of the ribosome such as tRNA and EF-G. This may support the fact that the relative movement between the two domains of RRF and the hydrophobic cleft are very important in the role of RRF in protein synthesis.

Therefore, since RRF is an essential protein for viability of bacteria(Janosi et al., 1994), RRF is an excellent target for the development of antibacterial drugs (Kaji et al., 1998) and the hydrophobic cleft of RRF is an excellent target for this purpose. Since the material binding to the hydrophobic cleft of RRF blocks the movement between the two domains of RRF, it likely blocks the ability of RRF to function in translation. Possible inhibitors can be easily screened and designed by using the three-dimensional structure of RRF according to the present invention. Ultimately, the material can be used as an RRF inhibitor and as an antibacterial agent.

The RRF crystal according to the present invention is obtained from the complex of RRF with decyl-β-D-maltopyranoside. This suggests that decyl-β-D-maltopyranoside is strongly bound to the hydrophobic cleft of RRF and inhibits its movement. Therefore, since decyl-β-D-maltopyranoside can inhibit the function of RRF, it can be used as an RRF inhibitor.

According to the three-dimensional structure of RRF analyzed from the RRF crystal with the detergent, seven carbon atoms on the alkyl chain of the detergent were found to be bound to the hydrophobic cleft of RRF. Seven of the carbon atoms from the terminus of the alkyl group are inserted into the hydrophobic cleft of RRF and the remaining three carbon atoms are outside of the cleft. The maltose group (disaccharide of 6 carbon) does not interact with the non-hydrophobic residues in the circumference of the hydrophobic cleft. Therefore, the carbon number in the hydrophobic alkyl group of the detergent binding to the cleft of RRF is preferably about 3 to 12. When the carbon number is smaller, the hydrophobic bonding is very weak. When the carbon number is larger, the part of alkyl group not binding to the cleft of RRF may act to repulse the other part of RRF. It is preferable for the detergent to have at least one group that can participate in non-hydrophobic bonding (hydrogen bonding, dipole-dipole bonding, charge-charge bonding and so on) with non-hydrophobic residues in the circumference of or near to the hydrophobic cleft of RRF. In this way, the hydrophobic alkyl group can play a role in binding to the cleft of RRF, and the non-hydrophobic group can provide the specific bonding with the non-hydrophobic amino acid residues in the circumference of or near to the cleft of RRF. In this way, the detergent can maintain high affinity binding with RRF.

Therefore, preferably the structure of a detergent for inhibiting the function of RRF includes an alkyl group with 3-12 carbon atoms in a straight chain and a non-hydrophobic group. The alkyl group of the detergent used in the present invention is preferably n-alkyl, but may be substituted with C1-C3 alkyl groups on at least one carbon of the straight chain.

Coiled Coil Domain of RRF binding to A-site of Ribosome

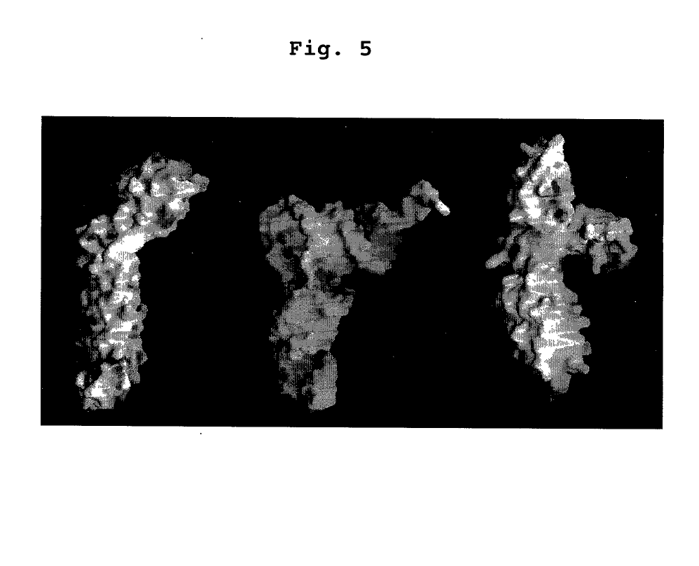

FIG. 5 provides the three-dimensional structural surface models of RRF and other molecules binding to the A-site of the ribosome (such as tRNA and EF-G). This shows the structural similarities therebetween. In the drawing, the model on the left is RRF, the center model is tRNA, and the right model is the 3,4,and 5 domains of EF-G. The white color indicates a hydrophobic surface. The red and blue colors indicate negatively and positively charged surfaces, respectively.

As shown in FIG. 5, the structure of RRF is similar to the structures of tRNA and EF-G, which bind to the A-site of the ribosome. In particular, the domain corresponding to the coiled coil of RRF has high structural similarity to the anticodon stem of tRNA and the domain IV of EF-G. From these and other results from several experiments, it is believed likely that the site directly binding to the A-site of the ribosome is on the coiled coil domain of RRF.

Therefore, the specific site binding to the A-site of the ribosome on the coiled coil domain can be identified using the three-dimensional structure of RRF according to the present invention and a material which binds to this specific site can be identified and designed. Such molecules may prevent RRF from binding to the A-site of the ribosome and can be used as an RRF inhibitor.

Identifying and Designing RRF inhibitors by Using the Three-Dimensional Structure of RRF

1) Identifying and Designing RRF inhibitors by Using the Hydrophobic Cleft of RRF

The present invention relates to a method for identifying and designing complementary molecules which bind to the hydrophobic cleft of RRF by using the three-dimensional structure, and synthesizing the molecule to use as an antibiotic. The complementary molecule can be predicted to bind to the cleft of RRF physically and structurally. The binding is not likely to be covalent, but can include hydrogen bonds, ionic bonds, van der Waals and hydrophobic interactions. The processes for identifying and designing RRF inhibitors are exemplified below.

a. The first step is to retrieve from a database, a molecule or its part which is predicted to bind to the hydrophobic cleft of RRF in the three-dimensional structure. Alternatively a molecule can be designed which, based on its three-dimensional structure, will bind to the cleft. The binding is not likely to be covalent, but may include hydrogen bonds, ion bonds, van der Waals and hydrophobic interactions. The compound or its part may also have a special conformation which allows it to bind the cleft. The conformation is not considered in the compound itself, but in the assumption of the compound binding with the cleft. Computer modeling can perform the selection and design of the compound or fragment. Further, prior to preparing the compound, the computer modeling technique can analyze or calculate the affinity between the compound or fragment and the cleft. If the computer modeling determines that the material or its part selected from the database or designed, does not have sufficient affinity, it is less likely to have RRF inhibitory ability and will not be used subsequently. The next processes include molecule remodeling for optimizing the affinity of the molecule to the cleft. This includes modifying the structure of the compound and exchanging substituents, preparing the remodeled compound and measuring the activity of the prepared compound. In this way, the efficiency of the compound as an RRF inhibitor may be increased, because it will exclude compounds which are not likely to have inhibitor activity. If computer modeling determines that the compound has sufficient affinity for the cleft of RRF, it will be prepared and the affinity will be measured in vitro as will its activity as an inhibitor. At this point, if the dissociation constant of the complex of the compound and RRF is 100 micromoles or less, the compound may be considered as a good inhibitor candidate. When the dissociation constant is 100 micromoles, 99% of the inhibitor exists in the complex and 1% of the remainder is dissociated from the complex. The less the dissociation constant is, the more the affinity of the compound to RRF is. In paragraph b below, the design of a stronger inhibitor based on the inhibitor candidate is described. The process for calculating the affinity of the compound for RRF by the use of a computer modeling technique is to bind the compound to the hydrophobic cleft of RRF by rotating it along several planes and to fit it to the cleft by visual inspection on the computer screen. This work may be performed with modeling programs such as Insight II, Sybyl, O, Chain and Frodo. The method for inserting the compound into the hydrophobic cleft of RRF may also be performed by docking, which may be performed using modeling programs such as Quanta, Autodock, Dock, Insight II, and Sybyl. The position of the compound in the binding site may be made to be more accurate by using energy minimization or molecular dynamics with standard molecular mechanics force fields such as CHARMM and AMBER.

Specialized computer programs may also perform the process of selecting the compound. These include:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method” Proteins: Structure, Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substates to Proteins by Simulated Annealing”, Proteins: Structure, Function and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982). DOCK is available from University of California, San Francisco, Calif.

When suitable chemical compounds or fragments are selected or designed and positioned in the binding site of the hydrophobic cleft, it may be possible and desirable to assemble them into a single compound. This may be accomplished using computer modeling programs as described above as well as the following programs:

1. CAVEAT (Bartlett, P. A. et al, “CAVEAT: A Program to Facilitate the Structure-Derived Design of Biologically Active Molecules” In “Molecular Recognition in Chemical and Biological Problems”, Special Pub., Royal Chem. Soc., 78, pp. 182-196 (1989). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

Once the material is designed or selected, the affinity of the material to RRF may be calculated. For the inhibitor to be effective, it should have a low energy difference between that energy calculated before and after binding. Thus, the most efficient inhibitor should preferably be designed with a deformation energy of binding of not greater than about 10 kcal/mole. The affinity of the inhibitor may also be measured by calculating the dissociation constant of the complex of RRF and the inhibitor. The dissociation constant is preferably 100 micromoles or less. The inhibitor preferably also maintains the bonding with RRF stably after binding. In order to do this, electrostatic repulsion such as charge-charge interactions, dipole-dipole and charge-dipole interactions between the inhibitor and RRF should not occur. The sum of electrostatic interaction should be neutral or give a positive effect to the enthalpy of the bonding. Examples of programs designed for calculating such affinity are as follows: Gaussian 92, revision C [M. J. Frisch, Gaussian, Inc., Pittsburgh, Pa. © 1992]; AMBER, version 4.0 [P. A. Kollman, University of California at San Fransisco, © 1994]; QUANTA/CHARMM [Molecular Simulations, Inc., Burlington, Mass. © 1994]; and Insight II/Discover (Biosysm Technologies Inc., San Diego, Calif., © 1994). Using the lead compound selected by the method, a stronger inhibitor can be made or designed. This process will be described below.

b. The compound (lead compound) selected or designed as above can be changed or modified. Atoms, substituents or a part of the structure may be altered to increase the binding affinity to RRF. Generally, initial substitutions are conservative, i.e., the replacement group will have approximately the same size, shape, hydrophobicity and charge as the original group. It should, of course, be understood that components known in the art to alter conformation should be avoided. The substituted chemical compounds may then be analyzed for fit with RRF by the same computer methods described in detail, above.

The present invention has identified a detergent, decyl-β-D-maltopyranoside, which was used for producing an RRF crystal by binding to the hydrophobic cleft of RRF. Therefore, decyl-β-D-maltopyranoside can be used as a lead compound for the design of RRF inhibitors by performing the same computer methods described above. If needed, an appropriate substituent may be introduced into the hydrophobic chain or alkyl chain of decyl-β-D-maltopyranoside. Alternatively, the maltose group may be changed to other appropriate groups.

c. After the material designed by the computer method described above is prepared and bound to RRF to produce a crystal, the three-dimensional structure of the complex may be determined at high resolution (over 0.28 nm) using X-ray crystallographic methods. The information gained about the interaction between RRF and the inhibitor obtained from this study may be used to modify the inhibitor and to increase the affinity of the inhibitor to RRF.

d. Once the inhibitor candidate for RRF optimized by this method has been selected or designed, the actual inhibitory activity of the candidate toward RRF may be tested in vitro and in vivo. When the inhibitor candidate is determined to have inhibitory activity toward RRF, it may then be used as an inhibitor. Alternatively, it may be used for developing a more optimal inhibitor based on its structure. For example, by improving properties such as its affinity to RRF and its solubility using the computer modeling method, the inhibitory activity may be optimized. In other words, the compound may be used as a lead compound for computer remodeling to increase the properties of RRF inhibition, solubility of the compound and so on. Before the compound is tested for inhibitory activity, the dissociation constant of the complex of the inhibitor and RRF may be measured experimentally in order to confirm the affinity calculated by the computer modeling method.

2) Identifying and Designing an RRF Inhibitor Based on the Three-Dimensional Structure of the RRF Domain While Binding to the A-Site of the Ribosome

a. Identifying the essential amino acid residues involved in binding to the ribosome. More specifically the amino acid residues in the coiled coil domain of RRF which bind to the A-site of the ribosome

The locations of amino acid residues in the surface of the coiled coil domain are as follows: 1, 3, 4, 6, 7, 8, 10, 11, 12, 14, 15, 17, 18, 19, 107, 108, 109, 111, 112, 114, 115, 116, 118, 119, 120, 122, 123, 125, 126, 127, 129, 130, 132, 133, 134, 136, 137, 138, 140, 141, 143, 144, 145, 146, 147, 148, 150, 151, 152, 153, 155, 156, 158, 159, 161, 162, 164, 165, 166, 167, 169, 170, 172, 173, 174, 176, 177, 179, 180, and 181. The amino acid residues are identified in the three-dimensional structure of RRF in FIG. 1 and the coordinates of the atoms in the structure of RRF are presented in table 2.

These atoms are considered to be involved in binding to the ribosome. RRF can be mutated by exchanging one or more of the amino acid residues on the coiled coil domain. If an E. coli possessing the mutated RRF does not grow well, grows slowly or not at all, the mutation has very likely affected the function of RRF. Specifically, the mutation has affected the ability of RRF to bind to the ribosome due to the substitution of the amino acid residue, and thus RRF cannot function in translation. This method can be used to identify amino acid residues in the original RRF which are important in the binding of RRF to the ribosome, more specifically to the A-site of the ribosome.

b. Identifying and designing an RRF inhibitor using the structure of the binding site in the coiled coil domain of RRF

Once the amino acid residues in the coiled coil domain of RRF have been identified as involved in the binding of RRF to the A-site of the ribosome, the structure of the binding site can be identified based on the three-dimensional structure of RRF. Based on the structure of the binding site, a compound such as a peptide or other compound can be screened and designed which will fit into the three-dimensional model of the binding site. For example, If a mutant which has the following residues substituted: Met-1, Ser-3 and Asp-4 has a very low activity, then these three residues are likely to be involved in the binding of RRF to the ribosome. A compound which is complementary to the structure of the binding site of RRF but includes these three residues would prevent RRF from binding to the ribosome. The complementary compound could be identified or designed from the structure of the binding site of RRF including the three residues.

The computer modeling method as described above can perform the identification and design of the complementary compounds. It can also be used to remodel the compound to improve the affinity or solubility, and produce an optimized compound.

The optimized compound may then be prepared and the inhibitory activity for RRF can be tested in vitro and in vivo. If the test confirms that the material does indeed inhibit RRF, then the material or a derivative can be used as an antibiotic. Using the method as described above, the compound identified to have inhibitory activity may be used as a lead compound to obtain an improved inhibitor.

In order to confirm the affinity predicted by the computer modeling method, the dissociation constant of the complex may be experimentally measured.

Identifying the Structure of the Complex of Inhibitor and RRF or Inhibitor and an RRF Homolog Using the Three-Dimensional Structure of RRF

The three-dimensional structure of RRF or one of its domains can be used to identify other crystal forms of RRF, the crystal structures of RRF homologs (those compounds having 30% amino acid similarity or more to RRF), the crystal structures of mutants of RRF or the crystal structure of a complex of RRF with another compound. Molecular replacement can be used for this. In order to identify the structure of the proteins as described above by this method, the coordinates of E. coli RRF (table 2) should be used. This method can quickly identify the unknown crystal structure. The programs used in the method are as follows:

CNS (Brger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. and Warren, G. L. (1998), Crystallography & NMR system: A New software suite for macromolecular structure determination, Acta Crystallogr., D 54, 905-921).

CCP4 program package (Collaborative Computational Project Number 4 (1994), The CCP4 suite: programs for protein crystallography, Acta crystallogr., D 50, 760-763); It includes molecular replacement program of Amore, Polarrfn et al.

EPMR (Kissinger, C. R., Gehlhaar, D. K. and Fogel, D. B. (1999), Rapid automated molecular replacement by evolutionary search, Acta Crystallogr., D 55, 484-491).

Development of an inhibitor which mimics the coiled coil domain of RRF

Since the coiled coil domain of RRF is involved in the binding with the A-site of the ribosome, a polypeptide which mimics a part or all of the coiled coil domain of RRF would also bind to the A-site of the ribosome. This polypeptide will compete with RRF for binding to the A-site—making it a competitive inhibitor. As a result, it would prevent RRF from binding to the ribosome. The preparation of a polypeptide which includes the entire coiled coil domain of RRF may be performed using general biological techniques or chemical synthesis based on the secondary structure and the three-dimensional structure of the amino acid sequence.

Although a peptide which includes the entire coiled coil domain of RRF can be used as an inhibitor for RRF, it is not likely to be efficient due to its large size. Therefore, it will be preferable to identify an inhibitor which is an amino acid or small polypeptide which has a structure which mimics the three-dimensional coiled coil structure of the binding site of RRF to the ribosome. As described above, the binding site can be identified by testing the viability of E. coli containing mutations in the coiled coil domain of RRF. Identification and design of the compound can be performed by computer modeling as described above. Further, for example, if a three-dimensional structure formed with two amino acids is identified as binding and a second three-dimensional structure with three-amino acids is also identified as binding near the first two-amino acid compound, then each of the peptides might be used as an inhibitor for RRF. In addition, a compound formed by appropriately linking the two peptides together might also be used as an inhibitor for RRF. The latter compound would be likely to have a higher affinity to RRF than the former peptides.

An alternative method for developing a compound such as an amino acid or polypeptide which has the identified structure of the binding domain is to identify the compound as above, and design or synthesize it. At this point, it would not be known if this compound will bind. The inhibitory activity of this compound against RRF will then be tested in vitro or in vivo. If the compound does have inhibitory activity against RRF, it can then be used as an inhibitor. This, then is another way of identifying the structure of the binding site in the coiled coil domain. Furthermore, when two or more compounds are identified which are located close to each other on the surface of the coiled coil domain, a new compound may be designed or synthesized by linking the two compounds. The new compound may then be tested as an inhibitor of RRF. A compound composed of two or more of these compounds will have a stronger inhibitory activity than each separate compound, because it can bind more tightly to the ribosome.

### Example 1

Expression and purification of E. coli RRF

The E. coli RRF gene was amplified by PCR using E. coli genomic DNA as a template. The PCR product was restricted and ligated into an NdeI/XhoI-restricted pET22b vector (Novagen). E. coli cells B834(DE3) were transformed with the pET22b-RRF plasmid and grown at 310° K in LB medium containing 50 μg ml⁻¹ ampicillin. E. coli RRF was induced with 0.5 mM IPTG for 4 h. The cells were harvested at 277 K by centrifugation (5000 g for 6 min) and resuspended in buffer A (20 mM Tris-HCl pH 8.0, 10 mM MgCl₂, 1 mM PMSF). The cells were then sonicated. After a subsequent centrifugation for 30 min at 15000×g, the supernatant was applied to a 5 ml HiTrap Q column (Pharmacia) equilibrated with buffer A. The flowthrough containing the RRF protein was dialyzed against buffer B (20 mM NaH₂PO₄ pH 6.0, 10 mM MgCl₂) and loaded onto a 5 ml HiTrap SP column (Pharmacia) equilibrated with buffer B. The protein identified in the flowthrough was concentrated by ultrafiltration (Amicon, YM 10) and loaded onto a Superdex 200 gel-filtration column (HiLoad 16/60, Pharmacia) equilibrated with buffer A containing 150 mM NaCl. The fractions containing RRF were pooled and concentrated for crystallization. The purity of RRF was checked by SDS gel electrophoresis.

### Example 2

Crystallization and Structural Analysis of E. coli RRF

A crystal of E. coli RRF was produced using polyethylene glycol as a precipitant in the presence of the detergent, decyl-β-D-maltopyranoside (Yun et al., 2000). The procedures were as follows: The RRF protein was prepared at a concentration of 100 mg/ml in a buffer (20 mM Tris-HCl, pH 8.0, 10 MM MgCl₂, 1 mM PMSF, 150 mM NaCl). The crystal growth solution had the composition: 0.1 M MES-NaOH, pH 6.5, 10% PEG 350 MME, and 12% PEG 400. 2 μl of the prepared protein solution was aliquoted onto a cover glass to form a droplet of about 2-3 mm diameter. Then 2 μl of the crystal growth solution and 0.4 μl of 18 mM decyl-β-D-maltopyranoside in turn were added to the droplet. The three solutions were not mixed other then allowing the natural mixing by diffusion. The final droplet with the three solutions was sealed, and equilibrated at 14° C. Vacuum grease was used for the sealant. Vapor diffusion of the solutions was begun in a closed system to equilibrate the solutions with the crystal growth solution and as a result, the concentration of the solution in the droplet was increased. Crystals were clustered and grew to dimensions of 0.15×0.15×1.0 mm within a week. When X-ray diffraction data from the crystal was collected and analyzed, it was found that the crystal had a unit cell of a=b=48.06 Å and c=142.27 Å, and a space group of P3₁21. The content of solvent was found to be 53.4%, when one molecule was in an asymmetric unit. To interpret the structure, multiple isomorphous replacement using anomalous scattering (MIRAS) and density modification such as solvent flipping was used. The programs DENZO and SCALEPACK were used for treating the X-ray diffraction data, the program “O” was used for modeling, and the program “CNS” for optimizing the model.

TABLE 2
The coordinates of the three-dimensional structure of RRF
analyzed from the RRF crystal of the present invention

| Atom type | Number | Average B | Maximum B | Average Q |
|---|---|---|---|---|
| Protein main chain | 740 | 50.506 | 108.570 | 1.000 |
| Protein side chain | 697 | 54.907 | 111.870 | 1.000 |
| Protein all atoms | 1437 | 52.640 | 111.870 | 1.000 |
| Ligand/substrate | 0 | 0.000 | 0.000 | 0.000 |
| Water molecules | 231 | 61.297 | 96.420 | 1.000 |
| Other entities | 14 | 65.118 | 79.610 | 1.000 |
| All atoms | 1682 | 53.933 | 111.870 | 1.000 |

| | | Atom | A.A. | A.A. | | | | | |
|---|---|---|---|---|---|---|---|---|---|
| Atom | No. | Type | Type | No. | X | Y | Z | OCC | B |
| ATOM | 1 | CB | MET | 1 | 0.523 | 6.682 | 82.996 | 1.00 | 100.83 |
| ATOM | 2 | CG | MET | 1 | 0.660 | 5.557 | 84.005 | 1.00 | 102.28 |
| ATOM | 3 | SD | MET | 1 | −0.459 | 5.752 | 85.397 | 1.00 | 104.69 |
| ATOM | 4 | CE | MET | 1 | 0.064 | 7.342 | 86.019 | 1.00 | 104.89 |
| ATOM | 5 | C | MET | 1 | −0.879 | 7.980 | 81.395 | 1.00 | 98.25 |
| ATOM | 6 | O | MET | 1 | −1.129 | 7.768 | 80.208 | 1.00 | 98.22 |
| ATOM | 7 | N | MET | 1 | −1.317 | 5.554 | 81.781 | 1.00 | 98.73 |
| ATOM | 8 | CA | MET | 1 | −0.883 | 6.836 | 82.409 | 1.00 | 99.14 |
| ATOM | 9 | N | ILE | 2 | −0.586 | 9.186 | 81.876 | 1.00 | 97.02 |
| ATOM | 10 | CA | ILE | 2 | −0.537 | 10.381 | 81.035 | 1.00 | 95.89 |
| ATOM | 11 | CB | ILE | 2 | −0.478 | 11.655 | 81.900 | 1.00 | 95.52 |
| ATOM | 12 | CG2 | ILE | 2 | −0.335 | 12.882 | 81.031 | 1.00 | 95.12 |
| ATOM | 13 | CG1 | ILE | 2 | −1.750 | 11.762 | 82.734 | 1.00 | 95.60 |
| ATOM | 14 | CD1 | ILE | 2 | −1.825 | 13.021 | 83.549 | 1.00 | 95.83 |
| ATOM | 15 | C | ILE | 2 | 0.661 | 10.372 | 80.086 | 1.00 | 95.21 |
| ATOM | 16 | O | ILE | 2 | 0.655 | 11.047 | 79.058 | 1.00 | 95.10 |
| ATOM | 17 | N | SER | 3 | 1.689 | 9.609 | 80.442 | 1.00 | 94.28 |
| ATOM | 18 | CA | SER | 3 | 2.889 | 9.501 | 79.619 | 1.00 | 93.89 |
| ATOM | 19 | CB | SER | 3 | 3.943 | 8.643 | 80.329 | 1.00 | 94.52 |
| ATOM | 20 | OG | SER | 3 | 4.245 | 9.152 | 81.617 | 1.00 | 95.52 |
| ATOM | 21 | C | SER | 3 | 2.503 | 8.822 | 78.311 | 1.00 | 92.84 |
| ATOM | 22 | O | SER | 3 | 2.949 | 9.203 | 77.229 | 1.00 | 92.71 |
| ATOM | 23 | N | ASP | 4 | 1.658 | 7.808 | 78.439 | 1.00 | 91.48 |
| ATOM | 24 | CA | ASP | 4 | 1.182 | 7.025 | 77.310 | 1.00 | 90.84 |
| ATOM | 25 | CB | ASP | 4 | 0.514 | 5.751 | 77.837 | 1.00 | 92.24 |
| ATOM | 26 | CO | ASP | 4 | 1.366 | 5.036 | 78.880 | 1.00 | 94.26 |
| ATOM | 27 | OD1 | ASP | 4 | 0.921 | 3.995 | 79.408 | 1.00 | 96.07 |
| ATOM | 28 | OD2 | ASP | 4 | 2.482 | 5.516 | 79.184 | 1.00 | 95.50 |
| ATOM | 29 | C | ASP | 4 | 0.186 | 7.836 | 76.472 | 1.00 | 88.80 |
| ATOM | 30 | O | ASP | 4 | 0.469 | 8.209 | 75.331 | 1.00 | 89.03 |
| ATOM | 31 | N | ILE | 5 | −0.972 | 8.102 | 77.067 | 1.00 | 85.55 |
| ATOM | 32 | CA | ILE | 5 | −2.054 | 8.859 | 76.448 | 1.00 | 81.93 |
| ATOM | 33 | CB | ILE | 5 | −2.902 | 9.566 | 77.535 | 1.00 | 80.24 |
| ATOM | 34 | CG2 | ILE | 5 | −3.969 | 10.453 | 76.893 | 1.00 | 79.36 |
| ATOM | 35 | CG1 | ILE | 5 | −3.549 | 8.519 | 78.439 | 1.00 | 77.95 |
| ATOM | 36 | CD1 | ILE | 5 | −4.442 | 9.097 | 79.495 | 1.00 | 76.67 |
| ATOM | 37 | C | ILE | 5 | −1.686 | 9.895 | 75.379 | 1.00 | 81.10 |
| ATOM | 38 | O | ILE | 5 | −2.162 | 9.803 | 74.247 | 1.00 | 80.52 |
| ATOM | 39 | N | ARG | 6 | −0.857 | 10.879 | 75.718 | 1.00 | 79.50 |
| ATOM | 40 | CA | ARG | 6 | −0.514 | 11.909 | 74.739 | 1.00 | 79.11 |
| ATOM | 41 | CB | ARG | 6 | 0.158 | 13.107 | 75.403 | 1.00 | 80.27 |
| ATOM | 42 | CG | ARG | 6 | 1.465 | 12.784 | 76.077 | 1.00 | 83.34 |
| ATOM | 43 | CD | ARG | 6 | 2.301 | 14.039 | 76.196 | 1.00 | 86.04 |
| ATOM | 44 | NE | ARG | 6 | 2.611 | 14.574 | 74.871 | 1.00 | 87.69 |
| ATOM | 45 | CZ | ARG | 6 | 3.314 | 15.679 | 74.659 | 1.00 | 87.77 |
| ATOM | 46 | NH1 | ARG | 6 | 3.780 | 16.374 | 75.692 | 1.00 | 88.33 |
| ATOM | 47 | NH2 | ARG | 6 | 3.546 | 16.086 | 73.418 | 1.00 | 85.84 |
| ATOM | 48 | C | ARG | 6 | 0.377 | 11.424 | 73.605 | 1.00 | 77.92 |
| ATOM | 49 | O | ARG | 6 | 0.228 | 11.866 | 72.467 | 1.00 | 77.98 |
| ATOM | 50 | N | LYS | 7 | 1.317 | 10.538 | 73.913 | 1.00 | 76.33 |
| ATOM | 51 | CA | LYS | 7 | 2.209 | 10.015 | 72.886 | 1.00 | 74.77 |
| ATOM | 52 | CB | LYS | 7 | 3.382 | 9.262 | 73.525 | 1.00 | 75.87 |
| ATOM | 53 | CG | LYS | 7 | 4.265 | 10.122 | 74.433 | 1.00 | 75.59 |
| ATOM | 54 | CD | LYS | 7 | 4.940 | 11.252 | 73.667 | 1.00 | 75.59 |
| ATOM | 55 | CE | LYS | 7 | 5.786 | 12.101 | 74.604 | 1.00 | 75.85 |
| ATOM | 56 | NZ | LYS | 7 | 6.601 | 13.132 | 73.900 | 1.00 | 74.95 |
| ATOM | 57 | C | LYS | 7 | 1.373 | 9.078 | 72.029 | 1.00 | 72.71 |
| ATOM | 58 | O | LYS | 7 | 1.693 | 8.805 | 70.877 | 1.00 | 72.89 |
| ATOM | 59 | N | ASP | 8 | 0.286 | 8.603 | 72.618 | 1.00 | 70.58 |
| ATOM | 60 | CA | ASP | 8 | −0.646 | 7.714 | 71.950 | 1.00 | 69.48 |
| ATOM | 61 | CS | ASP | 8 | −1.552 | 7.075 | 73.005 | 1.00 | 70.59 |
| ATOM | 62 | CG | ASP | 8 | −2.393 | 5.940 | 72.459 | 1.00 | 72.83 |
| ATOM | 63 | OD1 | ASP | 8 | −3.070 | 6.132 | 71.424 | 1.00 | 72.86 |
| ATOM | 64 | OD2 | ASP | 8 | −2.390 | 4.852 | 73.086 | 1.00 | 72.96 |
| ATOM | 65 | C | ASP | 8 | −1.480 | 8.575 | 70.981 | 1.00 | 68.88 |
| ATOM | 66 | O | ASP | 8 | −1.787 | 8.167 | 69.859 | 1.00 | 68.57 |
| ATOM | 67 | N | ALA | 9 | −1.833 | 9.776 | 71.428 | 1.00 | 66.22 |
| ATOM | 68 | CA | ALA | 9 | −2.633 | 10.689 | 70.631 | 1.00 | 63.64 |
| ATOM | 69 | CE | ALA | 9 | −3.115 | 11.854 | 71.494 | 1.00 | 61.48 |
| ATOM | 70 | C | ALA | 9 | −1.885 | 11.215 | 69.413 | 1.00 | 62.96 |
| ATOM | 71 | O | ALA | 9 | −2.455 | 11.287 | 68.323 | 1.00 | 63.12 |
| ATOM | 72 | N | GLU | 10 | −0.617 | 11.580 | 69.575 | 1.00 | 61.05 |
| ATOM | 73 | CA | GLU | 10 | 0.124 | 12.114 | 68.437 | 1.00 | 60.56 |
| ATOM | 74 | CB | GLU | 10 | 1.385 | 12.848 | 68.888 | 1.00 | 63.50 |
| ATOM | 75 | CC | GLU | 10 | 2.407 | 11.994 | 69.599 | 1.00 | 67.84 |
| ATOM | 76 | CD | GLU | 10 | 3.728 | 12.731 | 69.774 | 1.00 | 71.48 |
| ATOM | 77 | CE1 | GLU | 10 | 4.344 | 13.089 | 68.737 | 1.00 | 72.15 |
| ATOM | 78 | CE2 | GLU | 10 | 4.141 | 12.958 | 70.938 | 1.00 | 73.41 |
| ATOM | 79 | C | GLU | 10 | 0.487 | 11.041 | 67.422 | 1.00 | 57.27 |
| ATOM | 80 | O | GLU | 10 | 0.727 | 11.338 | 66.255 | 1.00 | 55.22 |
| ATOM | 81 | N | VAL | 11 | 0.549 | 9.794 | 67.863 | 1.00 | 55.19 |
| ATOM | 82 | CA | VAL | 11 | 0.839 | 8.728 | 66.922 | 1.00 | 53.74 |
| ATOM | 83 | CS | VAL | 11 | 1.232 | 7.412 | 67.629 | 1.00 | 54.76 |
| ATOM | 84 | CC1 | VAL | 11 | 1.279 | 6.261 | 66.616 | 1.00 | 54.05 |
| ATOM | 85 | CC2 | VAL | 11 | 2.608 | 7.576 | 68.265 | 1.00 | 54.99 |
| ATOM | 86 | C | VAL | 11 | −0.442 | 8.545 | 66.119 | 1.00 | 50.95 |
| ATOM | 87 | O | VAL | 11 | −0.408 | 8.228 | 64.935 | 1.00 | 51.54 |
| ATOM | 88 | N | ARG | 12 | −1.574 | 8.760 | 66.772 | 1.00 | 47.29 |
| ATOM | 89 | CA | ARG | 12 | −2.842 | 8.663 | 66.088 | 1.00 | 45.50 |
| ATOM | 90 | CB | ARG | 12 | −3.978 | 8.633 | 67.112 | 1.00 | 43.97 |
| ATOM | 91 | CC | ARG | 12 | −4.126 | 7.256 | 67.793 | 1.00 | 45.79 |
| ATOM | 92 | CD | ARG | 12 | −5.102 | 7.294 | 68.965 | 1.00 | 44.31 |
| ATOM | 93 | NE | ARG | 12 | −5.421 | 5.978 | 69.509 | 1.00 | 44.84 |
| ATOM | 94 | CZ | ARG | 12 | −6.083 | 5.776 | 70.653 | 1.00 | 43.11 |
| ATOM | 95 | NH1 | ARG | 12 | −6.492 | 6.794 | 71.379 | 1.00 | 42.34 |
| ATOM | 96 | NH2 | ARG | 12 | −6.339 | 4.550 | 71.080 | 1.00 | 45.09 |
| ATOM | 97 | C | ARG | 12 | −2.967 | 9.838 | 65.089 | 1.00 | 43.22 |
| ATOM | 98 | O | ARG | 12 | −3.217 | 9.627 | 63.908 | 1.00 | 40.86 |
| ATOM | 99 | N | MET | 13 | −2.732 | 11.063 | 65.543 | 1.00 | 43.08 |
| ATOM | 100 | CA | MET | 13 | −2.818 | 12.217 | 64.651 | 1.00 | 44.05 |
| ATOM | 101 | CB | MET | 13 | −2.617 | 13.519 | 65.421 | 1.00 | 43.78 |
| ATOM | 102 | CG | MET | 13 | −3.705 | 13.778 | 66.433 | 1.00 | 41.76 |
| ATOM | 103 | SD | MET | 13 | −3.401 | 15.313 | 67.300 | 1.00 | 47.04 |
| ATOM | 104 | CE | MET | 13 | −4.627 | 15.149 | 68.684 | 1.00 | 41.70 |
| ATOM | 105 | C | MET | 13 | −1.848 | 12.179 | 63.482 | 1.00 | 43.79 |
| ATOM | 106 | O | MET | 13 | −2.173 | 12.658 | 62.402 | 1.00 | 42.68 |
| ATOM | 107 | N | ASP | 14 | −0.662 | 11.619 | 63.700 | 1.00 | 45.44 |
| ATOM | 108 | CA | ASP | 14 | 0.337 | 11.507 | 62.641 | 1.00 | 47.26 |
| ATOM | 109 | CB | ASP | 14 | 1.665 | 10.990 | 63.209 | 1.00 | 52.47 |
| ATOM | 110 | CG | ASP | 14 | 2.658 | 12.101 | 63.429 | 1.00 | 55.85 |
| ATOM | 111 | OD1 | ASP | 14 | 3.764 | 11.830 | 63.938 | 1.00 | 60.98 |
| ATOM | 112 | OD2 | ASP | 14 | 2.331 | 13.254 | 63.075 | 1.00 | 57.16 |
| ATOM | 113 | C | ASP | 14 | −0.151 | 10.586 | 61.540 | 1.00 | 45.15 |
| ATOM | 114 | O | ASP | 14 | −0.008 | 10.867 | 60.358 | 1.00 | 45.21 |
| ATOM | 115 | N | LYE | 15 | −0.733 | 9.474 | 61.945 | 1.00 | 45.09 |
| ATOM | 116 | CA | LYE | 15 | −1.264 | 8.529 | 60.999 | 1.00 | 45.94 |
| ATOM | 117 | CE | LYE | 15 | −1.884 | 7.365 | 61.763 | 1.00 | 45.00 |
| ATOM | 118 | CG | LYE | 15 | −2.329 | 6.210 | 60.901 | 1.00 | 49.99 |
| ATOM | 119 | CD | LYE | 15 | −1.157 | 5.411 | 60.295 | 1.00 | 51.63 |
| ATOM | 120 | CE | LYE | 15 | −0.326 | 4.634 | 61.339 | 1.00 | 52.42 |
| ATOM | 121 | NZ | LYE | 15 | 0.435 | 5.471 | 62.309 | 1.00 | 52.22 |
| ATOM | 122 | C | LYE | 15 | −2.310 | 9.253 | 60.118 | 1.00 | 46.27 |
| ATOM | 123 | O | LYE | 15 | −2.313 | 9.094 | 58.889 | 1.00 | 48.65 |
| ATOM | 124 | N | CYS | 16 | −3.173 | 10.067 | 60.734 | 1.00 | 44.90 |
| ATOM | 125 | CA | CYS | 16 | −4.200 | 10.809 | 59.985 | 1.00 | 44.28 |
| ATOM | 126 | CE | CYE | 16 | −5.057 | 11.683 | 60.916 | 1.00 | 43.61 |
| ATOM | 127 | EG | CYS | 16 | −6.022 | 10.834 | 62.181 | 1.00 | 53.86 |
| ATOM | 128 | C | CYS | 16 | −3.604 | 11.730 | 58.908 | 1.00 | 41.26 |
| ATOM | 129 | O | CYS | 16 | −3.971 | 11.676 | 57.729 | 1.00 | 40.04 |
| ATOM | 130 | N | VAL | 17 | −2.697 | 12.596 | 59.324 | 1.00 | 38.60 |
| ATOM | 131 | CA | VAL | 17 | −2.091 | 13.512 | 58.384 | 1.00 | 38.03 |
| ATOM | 132 | CB | VAL | 17 | −1.082 | 14.435 | 59.092 | 1.00 | 39.03 |
| ATOM | 133 | CC1 | VAL | 17 | −0.512 | 15.452 | 58.111 | 1.00 | 38.47 |
| ATOM | 134 | CC2 | VAL | 17 | −1.766 | 15.144 | 60.257 | 1.00 | 38.78 |
| ATOM | 135 | C | VAL | 17 | −1.394 | 12.737 | 57.269 | 1.00 | 38.04 |
| ATOM | 136 | O | VAL | 17 | −1.530 | 13.074 | 56.095 | 1.00 | 35.00 |
| ATOM | 137 | N | GLU | 18 | −0.670 | 11.684 | 57.635 | 1.00 | 37.93 |
| ATOM | 138 | CA | GLU | 18 | 0.034 | 10.913 | 56.636 | 1.00 | 42.29 |
| ATOM | 139 | CE | GLU | 18 | 1.057 | 9.962 | 57.315 | 1.00 | 47.92 |
| ATOM | 140 | CC | GLU | 18 | 2.136 | 10.774 | 58.105 | 1.00 | 55.13 |
| ATOM | 141 | CD | GLU | 18 | 3.403 | 9.987 | 58.501 | 1.00 | 60.44 |
| ATOM | 142 | CE1 | GLU | 18 | 4.048 | 9.378 | 57.613 | 1.00 | 60.51 |
| ATOM | 143 | CE2 | GLU | 18 | 3.779 | 10.009 | 59.704 | 1.00 | 62.88 |
| ATOM | 144 | C | GLU | 18 | −0.935 | 10.205 | 55.687 | 1.00 | 41.27 |
| ATOM | 145 | 0 | GLU | 18 | −0.668 | 10.128 | 54.493 | 1.00 | 43.84 |
| ATOM | 146 | N | ALA | 19 | −2.072 | 9.726 | 56.187 | 1.00 | 38.68 |
| ATOM | 147 | CA | ALA | 19 | −3.060 | 9.110 | 55.300 | 1.00 | 37.55 |
| ATOM | 148 | CB | ALA | 19 | −4.241 | 8.521 | 56.116 | 1.00 | 35.72 |
| ATOM | 149 | C | ALA | 19 | −3.580 | 10.195 | 54.327 | 1.00 | 36.85 |
| ATOM | 150 | O | ALA | 19 | −3.802 | 9.937 | 53.133 | 1.00 | 39.34 |
| ATOM | 151 | N | PHE | 20 | −3.773 | 11.404 | 54.838 | 1.00 | 32.59 |
| ATOM | 152 | CA | PEE | 20 | −4.234 | 12.498 | 54.010 | 1.00 | 33.05 |
| ATOM | 153 | CB | PEE | 20 | −4.528 | 13.725 | 54.888 | 1.00 | 33.08 |
| ATOM | 154 | CC | PHE | 20 | −4.785 | 14.995 | 54.111 | 1.00 | 32.56 |
| ATOM | 155 | CD1 | PHE | 20 | −5.943 | 15.148 | 53.355 | 1.00 | 32.24 |
| ATOM | 156 | CD2 | PHE | 20 | −3.868 | 16.037 | 54.148 | 1.00 | 30.14 |
| ATOM | 157 | CE1 | PHE | 20 | −6.183 | 16.340 | 52.640 | 1.00 | 34.71 |
| ATOM | 158 | CE2 | PHE | 20 | −4.094 | 17.219 | 53.447 | 1.00 | 34.68 |
| ATOM | 159 | CZ | PHE | 20 | −5.257 | 17.375 | 52.689 | 1.00 | 32.40 |
| ATOM | 160 | C | PHE | 20 | −3.162 | 12.830 | 52.965 | 1.00 | 35.46 |
| ATOM | 161 | O | PHE | 20 | −3.460 | 13.017 | 51.772 | 1.00 | 36.29 |
| ATOM | 162 | N | LYS | 21 | −1.911 | 12.916 | 53.405 | 1.00 | 36.65 |
| ATOM | 163 | CA | LYS | 21 | −0.839 | 13.204 | 52.469 | 1.00 | 39.85 |
| ATOM | 164 | CB | LYS | 21 | 0.494 | 13.397 | 53.205 | 1.00 | 40.00 |
| ATOM | 165 | CC | LYS | 21 | 0.540 | 14.700 | 53.984 | 1.00 | 42.94 |
| ATOM | 166 | CD | LYS | 21 | 1.873 | 14.968 | 54.666 | 1.00 | 46.52 |
| ATOM | 167 | CE | LYS | 21 | 2.078 | 14.136 | 55.922 | 1.00 | 51.70 |
| ATOM | 168 | NZ | LYS | 21 | 2.091 | 12.659 | 55.699 | 1.00 | 56.00 |
| ATOM | 169 | C | LYS | 21 | −0.733 | 12.133 | 51.372 | 1.00 | 42.04 |
| ATOM | 170 | O | LYS | 21 | −0.480 | 12.477 | 50.221 | 1.00 | 42.08 |
| ATOM | 171 | N | THR | 22 | −0.966 | 10.855 | 51.686 | 1.00 | 44.06 |
| ATOM | 172 | CA | THR | 22 | −0.873 | 9.854 | 50.620 | 1.00 | 47.37 |
| ATOM | 173 | CB | THR | 22 | −0.619 | 8.362 | 51.126 | 1.00 | 46.50 |
| ATOM | 174 | OG1 | THR | 22 | −1.792 | 7.832 | 51.754 | 1.00 | 48.78 |
| ATOM | 175 | OG2 | THR | 22 | 0.524 | 8.314 | 52.105 | 1.00 | 45.25 |
| ATOM | 176 | C | THR | 22 | −2.141 | 9.863 | 49.787 | 1.00 | 47.95 |
| ATOM | 177 | O | THR | 22 | −2.091 | 9.686 | 48.569 | 1.00 | 50.97 |
| ATOM | 178 | N | GLN | 23 | −3.281 | 10.086 | 50.423 | 1.00 | 50.33 |
| ATOM | 179 | CA | GLN | 23 | −4.540 | 10.085 | 49.683 | 1.00 | 52.74 |
| ATOM | 180 | CB | GLN | 23 | −5.705 | 10.316 | 50.637 | 1.00 | 55.39 |
| ATOM | 181 | CC | GLN | 23 | −7.076 | 9.998 | 50.056 | 1.00 | 59.39 |
| ATOM | 182 | CD | GLN | 23 | −8.213 | 10.215 | 51.056 | 1.00 | 61.59 |
| ATOM | 183 | OE1 | GLN | 23 | −9.377 | 9.920 | 50.761 | 1.00 | 63.37 |
| ATOM | 184 | NE2 | GLN | 23 | −7.881 | 10.734 | 52.239 | 1.00 | 60.88 |
| ATOM | 185 | C | GLN | 23 | −4.549 | 11.159 | 48.598 | 1.00 | 55.03 |
| ATOM | 186 | O | GLN | 23 | −5.064 | 10.948 | 47.487 | 1.00 | 56.03 |
| ATOM | 187 | N | ILE | 24 | −3.946 | 12.299 | 48.923 | 1.00 | 54.26 |
| ATOM | 188 | CA | ILE | 24 | −3.891 | 13.443 | 48.032 | 1.00 | 54.96 |
| ATOM | 189 | CB | ILE | 24 | −3.712 | 14.733 | 48.888 | 1.00 | 56.27 |
| ATOM | 190 | CG2 | ILE | 24 | −2.378 | 14.711 | 49.601 | 1.00 | 57.16 |
| ATOM | 191 | CG1 | ILE | 24 | −3.785 | 15.976 | 48.019 | 1.00 | 56.46 |
| ATOM | 192 | CD1 | ILE | 24 | −3.556 | 17.244 | 48.803 | 1.00 | 57.81 |
| ATOM | 193 | C | ILE | 24 | −2.821 | 13.376 | 46.917 | 1.00 | 55.89 |
| ATOM | 194 | O | ILE | 24 | −3.043 | 13.870 | 45.808 | 1.00 | 53.59 |
| ATOM | 195 | N | SER | 25 | −1.672 | 12.759 | 47.188 | 1.00 | 56.28 |
| ATOM | 196 | CA | SER | 25 | −0.621 | 12.682 | 46.168 | 1.00 | 57.70 |
| ATOM | 197 | CE | SER | 25 | 0.647 | 12.031 | 46.734 | 1.00 | 56.60 |
| ATOM | 198 | OG | SER | 25 | 0.421 | 10.671 | 47.061 | 1.00 | 58.84 |
| ATOM | 199 | C | SER | 25 | −1.088 | 11.911 | 44.934 | 1.00 | 57.90 |
| ATOM | 200 | O | SER | 25 | −0.480 | 12.007 | 43.868 | 1.00 | 59.60 |
| ATOM | 201 | N | LYS | 26 | −2.167 | 11.147 | 45.081 | 1.00 | 57.63 |
| ATOM | 202 | CA | LYS | 26 | −2.723 | 10.384 | 43.975 | 1.00 | 57.26 |
| ATOM | 203 | CB | LYS | 26 | −3.825 | 9.454 | 44.474 | 1.00 | 58.98 |
| ATOM | 204 | CC | LYS | 26 | −3.353 | 8.353 | 45.412 | 1.00 | 62.43 |
| ATOM | 205 | CD | LYS | 26 | −4.504 | 7.416 | 45.750 | 1.00 | 64.95 |
| ATOM | 206 | CE | LYS | 26 | −4.044 | 6.265 | 46.633 | 1.00 | 67.37 |
| ATOM | 207 | NZ | LYS | 26 | −2.998 | 5.431 | 45.960 | 1.00 | 68.34 |
| ATOM | 208 | C | LYS | 26 | −3.303 | 11.297 | 42.899 | 1.00 | 57.18 |
| ATOM | 209 | O | LYS | 26 | −3.256 | 10.982 | 41.709 | 1.00 | 58.16 |
| ATOM | 210 | N | ILE | 27 | −3.845 | 12.431 | 43.330 | 1.00 | 55.61 |
| ATOM | 211 | CA | ILE | 27 | −4.457 | 13.411 | 42.436 | 1.00 | 54.60 |
| ATOM | 212 | CB | ILE | 27 | −5.180 | 14.523 | 43.250 | 1.00 | 54.29 |
| ATOM | 213 | CG2 | ILE | 27 | −5.451 | 15.72 | 642.360 | 1.00 | 54.06 |
| ATOM | 214 | CG1 | ILE | 27 | −6.456 | 13.96 | 743.878 | 1.00 | 51.10 |
| ATOM | 215 | OD1 | ILE | 27 | −6.215 | 12.81 | 844.784 | 1.00 | 49.52 |
| ATOM | 216 | C | ILE | 27 | −3.515 | 14.096 | 41.444 | 1.00 | 54.74 |
| ATOM | 217 | O | ILE | 27 | −2.557 | 14.776 | 41.825 | 1.00 | 52.96 |
| ATOM | 218 | N | ARG | 28 | −3.810 | 13.925 | 40.163 | 1.00 | 54.44 |
| ATOM | 219 | CA | ARG | 28 | −3.011 | 14.551 | 39.133 | 1.00 | 54.79 |
| ATOM | 220 | CD | ARG | 28 | −3.040 | 13.701 | 37.856 | 1.00 | 57.53 |
| ATOM | 221 | CC | ARG | 28 | −2.381 | 12.330 | 38.025 | 1.00 | 58.98 |
| ATOM | 222 | CO | ARG | 28 | −2.325 | 11.578 | 36.707 | 1.00 | 63.90 |
| ATOM | 223 | NE | ARG | 28 | −1.576 | 10.328 | 36.823 | 1.00 | 68.44 |
| ATOM | 224 | CZ | ARG | 28 | −1.298 | 9.509 | 35.808 | 1.00 | 69.24 |
| ATOM | 225 | NH1 | ARG | 28 | −1.709 | 9.801 | 34.575 | 1.00 | 69.86 |
| ATOM | 226 | NH2 | ARG | 28 | −0.608 | 8.394 | 36.027 | 1.00 | 67.68 |
| ATOM | 227 | C | ARG | 28 | −3.578 | 15.949 | 38.893 | 1.00 | 54.14 |
| ATOM | 228 | O | ARG | 28 | −4.795 | 16.154 | 38.889 | 1.00 | 55.27 |
| ATOM | 229 | N | THR | 29 | −2.698 | 16.915 | 38.705 | 1.00 | 51.89 |
| ATOM | 230 | CA | THR | 29 | −3.134 | 18.285 | 38.506 | 1.00 | 51.42 |
| ATOM | 231 | CD | THR | 29 | −2.414 | 19.187 | 39.502 | 1.00 | 51.85 |
| ATOM | 232 | OG1 | THR | 29 | −1.002 | 19.027 | 39.344 | 1.00 | 51.16 |
| ATOM | 233 | CG2 | THR | 29 | −2.786 | 18.798 | 40.924 | 1.00 | 51.25 |
| ATOM | 234 | C | THR | 29 | −2.918 | 18.810 | 37.082 | 1.00 | 50.96 |
| ATOM | 235 | O | THR | 29 | −3.337 | 19.926 | 36.752 | 1.00 | 50.77 |
| ATOM | 236 | N | GLY | 30 | −2.269 | 17.997 | 36.247 | 1.00 | 49.33 |
| ATOM | 237 | CA | GLY | 30 | −2.007 | 18.372 | 34.863 | 1.00 | 46.49 |
| ATOM | 238 | C | GLY | 30 | −2.275 | 17.181 | 33.963 | 1.00 | 44.01 |
| ATOM | 239 | O | GLY | 30 | −2.493 | 16.082 | 34.461 | 1.00 | 43.52 |
| ATOM | 240 | N | ARG | 31 | −2.256 | 17.386 | 32.649 | 1.00 | 42.12 |
| ATOM | 241 | CA | ARG | 31 | −2.520 | 16.301 | 31.703 | 1.00 | 40.50 |
| ATOM | 242 | CE | ARG | 31 | −2.980 | 16.873 | 30.361 | 1.00 | 41.80 |
| ATOM | 243 | CG | ARG | 31 | −4.281 | 17.585 | 30.412 | 1.00 | 41.56 |
| ATOM | 244 | CD | ARG | 31 | −4.707 | 18.090 | 29.040 | 1.00 | 42.11 |
| ATOM | 245 | NE | ARG | 31 | −6.018 | 18.713 | 29.146 | 1.00 | 42.16 |
| ATOM | 246 | CZ | ARG | 31 | −7.148 | 18.040 | 29.339 | 1.00 | 42.39 |
| ATOM | 247 | NH1 | ARG | 31 | −7.144 | 16.718 | 29.439 | 1.00 | 40.83 |
| ATOM | 248 | NH2 | ARG | 31 | −8.279 | 18.699 | 29.490 | 1.00 | 43.78 |
| ATOM | 249 | C | ARG | 31 | −1.350 | 15.347 | 31.440 | 1.00 | 38.97 |
| ATOM | 250 | O | ARG | 31 | −1.540 | 14.209 | 31.015 | 1.00 | 41.05 |
| ATOM | 251 | N | ALA | 32 | −0.143 | 15.812 | 31.698 | 1.00 | 37.59 |
| ATOM | 252 | CA | ALA | 32 | 1.059 | 15.033 | 31.454 | 1.00 | 36.56 |
| ATOM | 253 | CB | ALA | 32 | 2.278 | 15.811 | 31.987 | 1.00 | 36.73 |
| ATOM | 254 | C | ALA | 32 | 1.136 | 13.585 | 31.934 | 1.00 | 34.78 |
| ATOM | 255 | O | ALA | 32 | 0.995 | 13.299 | 33.114 | 1.00 | 37.37 |
| ATOM | 256 | N | SER | 33 | 1.397 | 12.678 | 31.001 | 1.00 | 34.58 |
| ATOM | 257 | CA | SER | 33 | 1.589 | 11.245 | 31.291 | 1.00 | 34.06 |
| ATOM | 258 | CB | SER | 33 | 0.253 | 10.507 | 31.463 | 1.00 | 36.06 |
| ATOM | 259 | OC | SER | 33 | 0.408 | 9.125 | 31.146 | 1.00 | 37.00 |
| ATOM | 260 | C | SER | 33 | 2.336 | 10.622 | 30.122 | 1.00 | 32.37 |
| ATOM | 261 | O | SER | 33 | 2.196 | 11.073 | 29.008 | 1.00 | 31.06 |
| ATOM | 262 | N | PRO | 34 | 3.192 | 9.621 | 30.377 | 1.00 | 32.68 |
| ATOM | 263 | CO | PRO | 34 | 3.664 | 9.098 | 31.670 | 1.00 | 33.36 |
| ATOM | 264 | CA | PRO | 34 | 3.924 | 8.970 | 29.285 | 1.00 | 34.44 |
| ATOM | 265 | CB | PRO | 34 | 4.871 | 8.021 | 30.029 | 1.00 | 34.92 |
| ATOM | 266 | CC | PRO | 34 | 4.085 | 7.690 | 31.284 | 1.00 | 34.26 |
| ATOM | 267 | C | PRO | 34 | 3.043 | 8.246 | 28.242 | 1.00 | 35.65 |
| ATOM | 268 | O | PRO | 34 | 3.463 | 8.094 | 27.097 | 1.00 | 36.81 |
| ATOM | 269 | N | SER | 35 | 1.829 | 7.824 | 28.613 | 1.00 | 34.52 |
| ATOM | 270 | CA | SER | 35 | 0.955 | 7.135 | 27.657 | 1.00 | 34.26 |
| ATOM | 271 | CR | SER | 35 | 0.267 | 5.925 | 28.308 | 1.00 | 33.31 |
| ATOM | 272 | OG | SER | 35 | −0.716 | 6.322 | 29.254 | 1.00 | 36.98 |
| ATOM | 273 | C | SER | 35 | −0.137 | 8.015 | 27.045 | 1.00 | 34.89 |
| ATOM | 274 | O | SER | 35 | −0.975 | 7.514 | 26.308 | 1.00 | 35.76 |
| ATOM | 275 | N | LEU | 36 | −0.120 | 9.313 | 27.334 | 1.00 | 34.17 |
| ATOM | 276 | CA | LEU | 36 | −1.148 | 10.248 | 26.835 | 1.00 | 34.56 |
| ATOM | 277 | CS | LEU | 36 | −0.771 | 11.700 | 27.195 | 1.00 | 35.25 |
| ATOM | 278 | CC | LEU | 36 | −1.881 | 12.732 | 26.941 | 1.00 | 33.58 |
| ATOM | 279 | CD1 | LEU | 36 | −3.008 | 12.435 | 27.923 | 1.00 | 35.46 |
| ATOM | 280 | CD2 | LEU | 36 | −1.383 | 14.168 | 27.139 | 1.00 | 33.44 |
| ATOM | 281 | C | LEU | 36 | −1.426 | 10.201 | 25.332 | 1.00 | 32.30 |
| ATOM | 282 | O | LEU | 36 | −2.562 | 10.295 | 24.891 | 1.00 | 29.65 |
| ATOM | 283 | N | LEC | 37 | −0.369 | 10.063 | 24.548 | 1.00 | 32.28 |
| ATOM | 284 | CA | LER | 37 | −0.500 | 10.055 | 23.103 | 1.00 | 30.09 |
| ATOM | 285 | CR | LEU | 37 | 0.567 | 10.983 | 22.509 | 1.00 | 34.71 |
| ATOM | 286 | CG | LEU | 37 | 0.618 | 12.421 | 23.042 | 1.00 | 36.56 |
| ATOM | 287 | CD1 | LEU | 37 | 1.836 | 13.144 | 22.442 | 1.00 | 41.17 |
| ATOM | 288 | CD2 | LEU | 37 | −0.691 | 13.157 | 22.714 | 1.00 | 35.03 |
| ATOM | 289 | C | LEU | 37 | −0.374 | 8.682 | 22.475 | 1.00 | 27.21 |
| ATOM | 290 | O | LEU | 37 | −0.263 | 8.578 | 21.263 | 1.00 | 26.50 |
| ATOM | 291 | N | ASP | 38 | −0.392 | 7.632 | 23.287 | 1.00 | 28.84 |
| ATOM | 292 | CA | ASP | 38 | −0.250 | 6.280 | 22.763 | 1.00 | 33.05 |
| ATOM | 293 | CR | ASP | 38 | −0.363 | 5.233 | 23.889 | 1.00 | 34.31 |
| ATOM | 294 | CC | ASP | 38 | 0.851 | 5.229 | 24.810 | 1.00 | 36.92 |
| ATOM | 295 | OD1 | ASP | 38 | 0.986 | 4.326 | 25.669 | 1.00 | 39.75 |
| ATOM | 296 | OD2 | ASP | 38 | 1.681 | 6.140 | 24.679 | 1.00 | 40.38 |
| ATOM | 297 | C | ASP | 38 | −1.228 | 5.942 | 21.630 | 1.00 | 34.44 |
| ATOM | 298 | O | ASP | 38 | −0.852 | 5.245 | 20.694 | 1.00 | 34.98 |
| ATOM | 299 | N | GLY | 39 | −2.452 | 6.465 | 21.713 | 1.00 | 34.31 |
| ATOM | 300 | CA | GLY | 39 | −3.478 | 6.194 | 20.719 | 1.00 | 34.90 |
| ATOM | 301 | CO | GLY | 39 | −3.412 | 6.898 | 19.370 | 1.00 | 35.63 |
| ATOM | 302 | O | GLY | 39 | −4.078 | 6.470 | 18.430 | 1.00 | 36.62 |
| ATOM | 303 | N | ILE | 40 | −2.634 | 7.967 | 19.253 | 1.00 | 35.51 |
| ATOM | 304 | CA | ILE | 40 | −2.510 | 8.667 | 17.972 | 1.00 | 35.87 |
| ATOM | 305 | CS | ILE | 40 | −1.503 | 9.862 | 18.082 | 1.00 | 36.81 |
| ATOM | 306 | CG2 | ILE | 40 | −1.254 | 10.491 | 16.712 | 1.00 | 33.51 |
| ATOM | 307 | CG1 | ILE | 40 | −2.061 | 10.913 | 19.047 | 1.00 | 37.34 |
| ATOM | 308 | CD1 | ILE | 40 | −1.192 | 12.161 | 19.151 | 1.00 | 36.05 |
| ATOM | 309 | C | ILE | 40 | −2.049 | 7.721 | 16.851 | 1.00 | 34.49 |
| ATOM | 310 | O | ILE | 40 | −1.033 | 7.048 | 16.979 | 1.00 | 32.65 |
| ATOM | 311 | N | VAL | 41 | −2.807 | 7.668 | 15.756 | 1.00 | 37.61 |
| ATOM | 312 | CA | VAL | 41 | −2.460 | 6.800 | 14.620 | 1.00 | 37.11 |
| ATOM | 313 | CR | VAL | 41 | −3.707 | 6.340 | 13.802 | 1.00 | 40.13 |
| ATOM | 314 | CR1 | VAL | 41 | −4.697 | 5.617 | 14.702 | 1.00 | 41.21 |
| ATOM | 315 | CR2 | VAL | 41 | −4.376 | 7.524 | 13.129 | 1.00 | 42.84 |
| ATOM | 316 | C | VAL | 41 | −1.522 | 7.521 | 13.669 | 1.00 | 35.61 |
| ATOM | 317 | O | VAL | 41 | −1.773 | 8.640 | 13.247 | 1.00 | 34.78 |
| ATOM | 318 | N | VAL | 42 | −0.430 | 6.867 | 13.330 | 1.00 | 37.16 |
| ATOM | 319 | CA | VAL | 42 | 0.546 | 7.462 | 12.444 | 1.00 | 39.92 |
| ATOM | 320 | CB | VAL | 42 | 1.926 | 7.532 | 13.136 | 1.00 | 37.79 |
| ATOM | 321 | CG1 | VAL | 42 | 2.927 | 8.217 | 12.232 | 1.00 | 37.61 |
| ATOM | 322 | CG2 | VAL | 42 | 1.809 | 8.259 | 14.460 | 1.00 | 37.43 |
| ATOM | 323 | C | VAL | 42 | 0.689 | 6.621 | 11.182 | 1.00 | 42.15 |
| ATOM | 324 | O | VAL | 42 | 0.644 | 5.395 | 11.245 | 1.00 | 40.81 |
| ATOM | 325 | N | GLU | 43 | 0.829 | 7.286 | 10.040 | 1.00 | 44.26 |
| ATOM | 326 | CA | GLU | 43 | 1.050 | 6.589 | 8.780 | 1.00 | 47.41 |
| ATOM | 327 | CS | GLU | 43 | 0.837 | 7.522 | 7.592 | 1.00 | 50.11 |
| ATOM | 328 | CG | GLU | 43 | −0.619 | 7.764 | 7.211 | 1.00 | 55.36 |
| ATOM | 329 | CD | GLU | 43 | −1.297 | 6.497 | 6.686 | 1.00 | 60.26 |
| ATOM | 330 | OE1 | GLU | 43 | −1.414 | 5.508 | 7.454 | 1.00 | 59.78 |
| ATOM | 331 | OE2 | GLU | 43 | −1.702 | 6.482 | 5.497 | 1.00 | 63.46 |
| ATOM | 332 | C | GLU | 43 | 2.496 | 6.121 | 8.800 | 1.00 | 48.82 |
| ATOM | 333 | O | GLU | 43 | 3.420 | 6.928 | 8.699 | 1.00 | 48.94 |
| ATOM | 334 | N | TYR | 44 | 2.691 | 4.817 | 8.943 | 1.00 | 50.69 |
| ATOM | 335 | CA | TYR | 44 | 4.032 | 4.237 | 8.999 | 1.00 | 52.56 |
| ATOM | 336 | CB | TYR | 44 | 4.193 | 3.521 | 10.337 | 1.00 | 51.42 |
| ATOM | 337 | CC | TYR | 44 | 5.560 | 2.957 | 10.626 | 1.00 | 51.48 |
| ATOM | 338 | CD1 | TYR | 44 | 6.670 | 3.791 | 10.773 | 1.00 | 49.46 |
| ATOM | 339 | CE1 | TYR | 44 | 7.919 | 3.268 | 11.107 | 1.00 | 49.00 |
| ATOM | 340 | CD2 | TYR | 44 | 5.739 | 1.581 | 10.811 | 1.00 | 50.45 |
| ATOM | 341 | CR2 | TYR | 44 | 6.982 | 1.055 | 11.143 | 1.00 | 49.22 |
| ATOM | 342 | CZ | TYR | 44 | 8.064 | 1.898 | 11.295 | 1.00 | 48.06 |
| ATOM | 343 | OH | TYR | 44 | 9.268 | 1.366 | 11.682 | 1.00 | 47.29 |
| ATOM | 344 | C | TYR | 44 | 4.165 | 3.263 | 7.832 | 1.00 | 54.71 |
| ATOM | 345 | O | TYR | 44 | 3.643 | 2.147 | 7.873 | 1.00 | 55.85 |
| ATOM | 346 | N | TYR | 45 | 4.864 | 3.708 | 6.790 | 1.00 | 58.86 |
| ATOM | 347 | CA | TYR | 45 | 5.067 | 2.941 | 5.552 | 1.00 | 60.22 |
| ATOM | 348 | CB | TYR | 45 | 6.152 | 1.879 | 5.721 | 1.00 | 60.58 |
| ATOM | 349 | CC | TYR | 45 | 7.497 | 2.434 | 6.123 | 1.00 | 60.52 |
| ATOM | 350 | CD1 | TYR | 45 | 7.701 | 2.944 | 7.400 | 1.00 | 60.80 |
| ATOM | 351 | CE1 | TYR | 45 | 8.937 | 3.464 | 7.780 | 1.00 | 61.27 |
| ATOM | 352 | CD2 | TYR | 45 | 8.564 | 2.458 | 5.226 | 1.00 | 59.56 |
| ATOM | 353 | CE2 | TYR | 45 | 9.801 | 2.977 | 5.596 | 1.00 | 61.01 |
| ATOM | 354 | CZ | TYR | 45 | 9.979 | 3.482 | 6.881 | 1.00 | 60.45 |
| ATOM | 355 | OH | TYR | 45 | 11.185 | 4.030 | 7.276 | 1.00 | 62.38 |
| ATOM | 356 | C | TYR | 45 | 3.782 | 2.280 | 5.076 | 1.00 | 61.45 |
| ATOM | 357 | O | TYR | 45 | 3.724 | 1.064 | 4.912 | 1.00 | 61.25 |
| ATOM | 358 | N | GLY | 46 | 2.751 | 3.100 | 4.873 | 1.00 | 64.05 |
| ATOM | 359 | CA | GLY | 46 | 1.461 | 2.608 | 4.407 | 1.00 | 65.41 |
| ATOM | 360 | C | GLY | 46 | 0.458 | 2.193 | 5.473 | 1.00 | 66.35 |
| ATOM | 361 | O | GLY | 46 | −0.737 | 2.509 | 5.368 | 1.00 | 66.56 |
| ATOM | 362 | N | THR | 47 | 0.952 | 1.501 | 6.501 | 1.00 | 66.38 |
| ATOM | 363 | CA | THR | 47 | 0.126 | 0.982 | 7.594 | 1.00 | 65.82 |
| ATOM | 364 | CB | THR | 47 | 0.711 | −0.347 | 8.135 | 1.00 | 67.46 |
| ATOM | 365 | OG1 | THR | 47 | 2.011 | −0.101 | 8.693 | 1.00 | 69.00 |
| ATOM | 366 | OG2 | THR | 47 | 0.842 | −1.375 | 7.012 | 1.00 | 67.57 |
| ATOM | 367 | C | THR | 47 | −0.058 | 1.920 | 8.789 | 1.00 | 63.78 |
| ATOM | 368 | O | THR | 47 | 0.912 | 2.391 | 9.393 | 1.00 | 63.80 |
| ATOM | 369 | N | PRO | 48 | −1.317 | 2.208 | 9.145 | 1.00 | 61.42 |
| ATOM | 370 | CD | PRO | 48 | −2.587 | 1.799 | 8.522 | 1.00 | 59.86 |
| ATOM | 371 | CA | PRO | 48 | −1.577 | 3.092 | 10.286 | 1.00 | 58.41 |
| ATOM | 372 | CE | PRO | 48 | −3.097 | 3.296 | 10.217 | 1.00 | 58.67 |
| ATOM | 373 | CC | PRO | 48 | −3.403 | 3.068 | 8.701 | 1.00 | 59.94 |
| ATOM | 374 | C | PRO | 48 | −1.137 | 2.360 | 11.550 | 1.00 | 54.30 |
| ATOM | 375 | O | PRO | 48 | −1.545 | 1.228 | 11.790 | 1.00 | 53.93 |
| ATOM | 376 | N | THR | 49 | −0.287 | 2.994 | 12.344 | 1.00 | 49.82 |
| ATOM | 377 | CA | THR | 49 | 0.191 | 2.363 | 13.560 | 1.00 | 45.54 |
| ATOM | 378 | CE | THR | 49 | 1.626 | 1.811 | 13.384 | 1.00 | 45.94 |
| ATOM | 379 | OG1 | THR | 49 | 2.548 | 2.895 | 13.261 | 1.00 | 51.01 |
| ATOM | 380 | OG2 | THR | 49 | 1.724 | 0.975 | 12.129 | 1.00 | 44.44 |
| ATOM | 381 | C | THR | 49 | 0.167 | 3.344 | 14.723 | 1.00 | 41.67 |
| ATOM | 382 | O | THR | 49 | 0.462 | 4.526 | 14.563 | 1.00 | 39.81 |
| ATOM | 383 | N | PRO | 50 | −0.207 | 2.861 | 15.915 | 1.00 | 38.96 |
| ATOM | 384 | CD | PRO | 50 | −0.608 | 1.492 | 16.275 | 1.00 | 36.47 |
| ATOM | 385 | CA | PRO | 50 | −0.258 | 3.731 | 17.087 | 1.00 | 35.39 |
| ATOM | 386 | CB | PRO | 50 | −0.768 | 2.787 | 18.189 | 1.00 | 35.03 |
| ATOM | 387 | CG | PRO | 50 | −1.589 | 1.770 | 17.391 | 1.00 | 37.74 |
| ATOM | 388 | C | PRO | 50 | 1.134 | 4.282 | 17.372 | 1.00 | 34.29 |
| ATOM | 389 | O | PRO | 50 | 2.132 | 3.566 | 17.222 | 1.00 | 32.88 |
| ATOM | 390 | N | LEO | 51 | 1.177 | 5.553 | 17.777 | 1.00 | 32.65 |
| ATOM | 391 | CA | LEU | 51 | 2.408 | 6.261 | 18.106 | 1.00 | 32.71 |
| ATOM | 392 | CB | LEU | 51 | 2.057 | 7.654 | 18.627 | 1.00 | 32.91 |
| ATOM | 393 | CG | LEU | 51 | 3.183 | 8.543 | 19.140 | 1.00 | 32.51 |
| ATOM | 394 | CD1 | LEU | 51 | 4.317 | 8.566 | 18.117 | 1.00 | 35.14 |
| ATOM | 395 | CD2 | LEU | 51 | 2.644 | 9.950 | 19.354 | 1.00 | 34.75 |
| ATOM | 396 | C | LEU | 51 | 3.259 | 5.527 | 19.146 | 1.00 | 35.19 |
| ATOM | 397 | O | LEU | 51 | 4.500 | 5.620 | 19.153 | 1.00 | 32.93 |
| ATOM | 398 | N | ARG | 52 | 2.573 | 4.817 | 20.036 | 1.00 | 35.13 |
| ATOM | 399 | CA | ARG | 52 | 3.205 | 4.055 | 21.102 | 1.00 | 35.65 |
| ATOM | 400 | CB | ARG | 52 | 2.122 | 3.263 | 21.853 | 1.00 | 35.45 |
| ATOM | 401 | CG | ARG | 52 | 2.629 | 2.303 | 22.934 | 1.00 | 37.41 |
| ATOM | 402 | CO | ARG | 52 | 1.456 | 1.582 | 23.582 | 1.00 | 39.06 |
| ATOM | 403 | NE | ARG | 52 | 0.576 | 1.021 | 22.547 | 1.00 | 44.41 |
| ATOM | 404 | CZ | ARG | 52 | 0.722 | −0.168 | 21.949 | 1.00 | 44.42 |
| ATOM | 405 | NE1 | ARG | 52 | 1.726 | −0.983 | 22.266 | 1.00 | 41.83 |
| ATOM | 406 | NH2 | ARG | 52 | −0.138 | −0.526 | 21.005 | 1.00 | 42.08 |
| ATOM | 407 | C | ARG | 52 | 4.299 | 3.103 | 20.586 | 1.00 | 34.33 |
| ATOM | 408 | O | ARG | 52 | 5.304 | 2.914 | 21.241 | 1.00 | 35.09 |
| ATOM | 409 | N | GLN | 53 | 4.092 | 2.519 | 19.417 | 1.00 | 34.91 |
| ATOM | 410 | CA | GLN | 53 | 5.049 | 1.572 | 18.837 | 1.00 | 38.97 |
| ATOM | 411 | CE | GLN | 53 | 4.322 | 0.539 | 17.959 | 1.00 | 40.17 |
| ATOM | 412 | CC | GLN | 53 | 3.235 | −0.225 | 18.663 | 1.00 | 46.58 |
| ATOM | 413 | CD | GLN | 53 | 2.466 | −1.151 | 17.736 | 1.00 | 50.36 |
| ATOM | 414 | OE1 | GLN | 53 | 1.897 | −0.727 | 16.727 | 1.00 | 50.89 |
| ATOM | 415 | NE2 | GLN | 53 | 2.429 | −2.431 | 18.090 | 1.00 | 55.52 |
| ATOM | 416 | C | GLN | 53 | 6.111 | 2.244 | 17.966 | 1.00 | 39.42 |
| ATOM | 417 | O | GLN | 53 | 6.975 | 1.577 | 17.447 | 1.00 | 36.91 |
| ATOM | 418 | N | LEU | 54 | 6.033 | 3.560 | 17.808 | 1.00 | 40.51 |
| ATOM | 419 | CA | LEU | 54 | 6.967 | 4.272 | 16.963 | 1.00 | 40.80 |
| ATOM | 420 | CB | LEU | 54 | 6.179 | 4.999 | 15.868 | 1.00 | 39.66 |
| ATOM | 421 | CC | LEU | 54 | 5.261 | 4.086 | 15.042 | 1.00 | 44.30 |
| ATOM | 422 | CD1 | LEU | 54 | 4.362 | 4.929 | 14.106 | 1.00 | 42.27 |
| ATOM | 423 | CD2 | LEU | 54 | 6.102 | 3.085 | 14.243 | 1.00 | 40.97 |
| ATOM | 424 | C | LEU | 54 | 7.823 | 5.269 | 17.739 | 1.00 | 40.78 |
| ATOM | 425 | O | LEU | 54 | 8.772 | 5.817 | 17.214 | 1.00 | 41.97 |
| ATOM | 426 | N | ALA | 55 | 7.506 | 5.510 | 18.998 | 1.00 | 40.71 |
| ATOM | 427 | CA | ALA | 55 | 8.285 | 6.485 | 19.734 | 1.00 | 40.64 |
| ATOM | 428 | CB | ALA | 55 | 7.855 | 7.889 | 19.332 | 1.00 | 38.83 |
| ATOM | 429 | C | ALA | 55 | 8.180 | 6.352 | 21.232 | 1.00 | 40.46 |
| ATOM | 430 | O | ALA | 55 | 7.256 | 5.747 | 21.759 | 1.00 | 40.59 |
| ATOM | 431 | N | SER | 56 | 9.166 | 6.927 | 21.898 | 1.00 | 41.32 |
| ATOM | 432 | CA | SER | 56 | 9.243 | 6.982 | 23.346 | 1.00 | 41.03 |
| ATOM | 433 | CE | SER | 56 | 10.715 | 6.932 | 23.771 | 1.00 | 44.77 |
| ATOM | 434 | CC | SER | 56 | 10.860 | 7.109 | 25.169 | 1.00 | 49.29 |
| ATOM | 435 | C | SER | 56 | 8.635 | 8.339 | 23.718 | 1.00 | 39.72 |
| ATOM | 436 | O | SER | 56 | 9.024 | 9.365 | 23.151 | 1.00 | 37.62 |
| ATOM | 437 | N | VAL | 57 | 7.667 | 8.350 | 24.634 | 1.00 | 40.18 |
| ATOM | 438 | CA | VAL | 57 | 7.041 | 9.605 | 25.059 | 1.00 | 38.81 |
| ATOM | 439 | CB | VAL | 57 | 5.503 | 9.601 | 24.817 | 1.00 | 38.08 |
| ATOM | 440 | CG1 | VAL | 57 | 4.885 | 10.935 | 25.240 | 1.00 | 35.66 |
| ATOM | 441 | CG2 | VAL | 57 | 5.218 | 9.368 | 23.360 | 1.00 | 34.50 |
| ATOM | 442 | C | VAL | 57 | 7.334 | 9.821 | 26.530 | 1.00 | 41.07 |
| ATOM | 443 | O | VAL | 57 | 6.827 | 9.096 | 27.386 | 1.00 | 43.17 |
| ATOM | 444 | N | THR | 58 | 8.159 | 10.828 | 26.814 | 1.00 | 42.76 |
| ATOM | 445 | CA | THR | 58 | 8.577 | 11.160 | 28.173 | 1.00 | 44.28 |
| ATOM | 446 | CB | THR | 58 | 10.109 | 11.316 | 28.251 | 1.00 | 44.97 |
| ATOM | 447 | OG1 | THR | 58 | 10.730 | 10.029 | 28.151 | 1.00 | 50.84 |
| ATOM | 448 | OG2 | THR | 58 | 10.522 | 11.948 | 29.563 | 1.00 | 50.51 |
| ATOM | 449 | C | THR | 58 | 7.953 | 12.444 | 28.670 | 1.00 | 46.01 |
| ATOM | 450 | O | THR | 58 | 7.716 | 13.373 | 27.888 | 1.00 | 47.72 |
| ATOM | 451 | N | VAL | 59 | 7.689 | 12.516 | 29.971 | 1.00 | 45.39 |
| ATOM | 452 | CA | VAL | 59 | 7.107 | 13.736 | 30.522 | 1.00 | 45.59 |
| ATOM | 453 | CB | VAL | 59 | 6.336 | 13.468 | 31.829 | 1.00 | 42.70 |
| ATOM | 454 | CG1 | VAL | 59 | 5.726 | 14.768 | 32.327 | 1.00 | 42.59 |
| ATOM | 455 | CG2 | VAL | 59 | 5.272 | 12.408 | 31.613 | 1.00 | 41.35 |
| ATOM | 456 | C | VAL | 59 | 8.218 | 14.753 | 30.809 | 1.00 | 48.24 |
| ATOM | 457 | O | VAL | 59 | 8.936 | 14.645 | 31.801 | 1.00 | 44.55 |
| ATOM | 458 | N | GLU | 60 | 8.369 | 15.736 | 29.933 | 1.00 | 48.24 |
| ATOM | 459 | CA | GLU | 60 | 9.400 | 16.740 | 30.136 | 1.00 | 50.27 |
| ATOM | 460 | CE | GLU | 60 | 9.559 | 17.588 | 28.868 | 1.00 | 50.29 |
| ATOM | 461 | CC | GLU | 60 | 10.513 | 18.765 | 28.996 | 1.00 | 53.37 |
| ATOM | 462 | CD | GLU | 60 | 10.795 | 19.442 | 27.659 | 1.00 | 53.53 |
| ATOM | 463 | OE1 | GLU | 60 | 11.657 | 18.958 | 26.912 | 1.00 | 56.23 |
| ATOM | 464 | OE2 | GLU | 60 | 10.145 | 20.453 | 27.336 | 1.00 | 55.58 |
| ATOM | 465 | C | GLU | 60 | 9.004 | 17.575 | 31.357 | 1.00 | 51.63 |
| ATOM | 466 | O | GLU | 60 | 9.825 | 17.863 | 32.216 | 1.00 | 51.46 |
| ATOM | 467 | N | ASP | 61 | 7.730 | 17.927 | 31.445 | 1.00 | 53.42 |
| ATOM | 468 | CA | ASP | 61 | 7.225 | 18.694 | 32.579 | 1.00 | 56.91 |
| ATOM | 469 | OB | ASP | 61 | 7.567 | 20.176 | 32.418 | 1.00 | 57.87 |
| ATOM | 470 | CG | ASP | 61 | 7.065 | 20.736 | 31.115 | 1.00 | 59.93 |
| ATOM | 471 | OD1 | ASP | 61 | 7.636 | 20.363 | 30.059 | 1.00 | 60.62 |
| ATOM | 472 | OD2 | ASP | 61 | 6.085 | 21.520 | 31.144 | 1.00 | 61.00 |
| ATOM | 473 | C | ASP | 61 | 5.701 | 18.531 | 32.694 | 1.00 | 57.24 |
| ATOM | 474 | O | ASP | 61 | 5.055 | 18.036 | 31.775 | 1.00 | 59.19 |
| ATOM | 475 | N | SER | 62 | 5.146 | 18.967 | 33.821 | 1.00 | 57.11 |
| ATOM | 476 | CA | SER | 62 | 3.714 | 18.893 | 34.120 | 1.00 | 56.21 |
| ATOM | 477 | CB | SER | 62 | 3.404 | 19.839 | 35.267 | 1.00 | 58.84 |
| ATOM | 478 | OG | SER | 62 | 3.730 | 21.172 | 34.891 | 1.00 | 61.16 |
| ATOM | 479 | C | SER | 62 | 2.727 | 19.186 | 32.980 | 1.00 | 54.83 |
| ATOM | 480 | O | SER | 62 | 1.606 | 18.675 | 32.988 | 1.00 | 54.24 |
| ATOM | 481 | N | ARG | 63 | 3.115 | 20.019 | 32.022 | 1.00 | 52.32 |
| ATOM | 482 | CA | ARG | 63 | 2.214 | 20.326 | 30.920 | 1.00 | 51.50 |
| ATOM | 483 | CE | ARG | 63 | 1.575 | 21.711 | 31.112 | 1.00 | 53.61 |
| ATOM | 484 | CG | ARG | 63 | 2.516 | 22.912 | 31.044 | 1.00 | 57.29 |
| ATOM | 485 | CD | ARG | 63 | 1.906 | 24.083 | 31.836 | 1.00 | 60.21 |
| ATOM | 486 | NE | ARG | 63 | 2.647 | 25.338 | 31.717 | 1.00 | 62.28 |
| ATOM | 487 | CZ | ARG | 63 | 2.565 | 26.160 | 30.675 | 1.00 | 65.53 |
| ATOM | 488 | NE1 | ARG | 63 | 1.767 | 25.860 | 29.651 | 1.00 | 66.70 |
| ATOM | 489 | NH2 | ARG | 63 | 3.272 | 27.289 | 30.657 | 1.00 | 65.73 |
| ATOM | 490 | C | ARG | 63 | 2.893 | 20.241 | 29.562 | 1.00 | 48.65 |
| ATOM | 491 | O | ARG | 63 | 2.458 | 20.872 | 28.606 | 1.00 | 49.55 |
| ATOM | 492 | N | THR | 64 | 3.946 | 19.438 | 29.473 | 1.00 | 44.74 |
| ATOM | 493 | CA | THR | 64 | 4.659 | 19.298 | 28.215 | 1.00 | 43.06 |
| ATOM | 494 | CB | THR | 64 | 5.827 | 20.305 | 28.134 | 1.00 | 42.31 |
| ATOM | 495 | OG1 | THR | 64 | 5.337 | 21.616 | 28.443 | 1.00 | 46.00 |
| ATOM | 496 | OG2 | THR | 64 | 6.440 | 20.321 | 26.726 | 1.00 | 41.82 |
| ATOM | 497 | C | THR | 64 | 5.215 | 17.884 | 27.983 | 1.00 | 41.37 |
| ATOM | 498 | O | THR | 64 | 5.806 | 17.270 | 28.888 | 1.00 | 38.27 |
| ATOM | 499 | N | LED | 65 | 5.010 | 17.363 | 26.777 | 1.00 | 37.51 |
| ATOM | 500 | CA | LED | 65 | 5.540 | 16.050 | 26.464 | 1.00 | 38.11 |
| ATOM | 501 | CB | LED | 65 | 4.477 | 15.102 | 25.875 | 1.00 | 34.91 |
| ATOM | 502 | CC | LED | 65 | 3.255 | 14.797 | 26.745 | 1.00 | 35.31 |
| ATOM | 503 | CD1 | LEU | 65 | 2.412 | 13.721 | 26.098 | 1.00 | 33.78 |
| ATOM | 504 | CD2 | LEU | 65 | 3.712 | 14.367 | 28.108 | 1.00 | 34.37 |
| ATOM | 505 | C | LEU | 65 | 6.669 | 16.170 | 25.473 | 1.00 | 38.28 |
| ATOM | 506 | O | LEU | 65 | 6.685 | 17.051 | 24.614 | 1.00 | 38.59 |
| ATOM | 507 | N | LYS | 66 | 7.615 | 15.254 | 25.613 | 1.00 | 40.15 |
| ATOM | 508 | CA | LYS | 66 | 8.774 | 15.161 | 24.743 | 1.00 | 40.02 |
| ATOM | 509 | CE | LYS | 66 | 10.043 | 15.189 | 25.588 | 1.00 | 45.80 |
| ATOM | 510 | CO | LYS | 66 | 11.202 | 14.454 | 24.977 | 1.00 | 51.87 |
| ATOM | 511 | CD | LYS | 66 | 12.272 | 14.216 | 26.026 | 1.00 | 56.23 |
| ATOM | 512 | CE | LYS | 66 | 13.346 | 13.286 | 25.476 | 1.00 | 59.78 |
| ATOM | 513 | NZ | LYS | 66 | 14.455 | 13.197 | 26.441 | 1.00 | 60.71 |
| ATOM | 514 | C | LYS | 66 | 8.609 | 13.823 | 24.031 | 1.00 | 37.57 |
| ATOM | 515 | O | LYS | 66 | 8.606 | 12.770 | 24.668 | 1.00 | 33.81 |
| ATOM | 516 | N | ILE | 67 | 8.474 | 13.871 | 22.715 | 1.00 | 36.18 |
| ATOM | 517 | CA | ILE | 67 | 8.313 | 12.666 | 21.923 | 1.00 | 37.12 |
| ATOM | 518 | CB | ILE | 67 | 7.241 | 12.841 | 20.853 | 1.00 | 36.65 |
| ATOM | 519 | CG2 | ILE | 67 | 7.057 | 11.546 | 20.108 | 1.00 | 39.63 |
| ATOM | 520 | CG1 | ILE | 67 | 5.911 | 13.234 | 21.473 | 1.00 | 34.46 |
| ATOM | 521 | CD1 | ILE | 67 | 4.873 | 13.466 | 20.423 | 1.00 | 35.05 |
| ATOM | 522 | C | ILE | 67 | 9.624 | 12.276 | 21.246 | 1.00 | 39.04 |
| ATOM | 523 | O | ILE | 67 | 10.195 | 13.035 | 20.466 | 1.00 | 39.66 |
| ATOM | 524 | N | ASN | 68 | 10.086 | 11.075 | 21.539 | 1.00 | 40.32 |
| ATOM | 525 | CA | ASN | 68 | 11.331 | 10.606 | 20.975 | 1.00 | 41.37 |
| ATOM | 526 | CB | ASN | 68 | 12.197 | 10.115 | 22.119 | 1.00 | 44.76 |
| ATOM | 527 | CC | ASN | 68 | 13.584 | 9.842 | 21.691 | 1.00 | 47.81 |
| ATOM | 528 | OD1 | ASN | 68 | 14.155 | 10.636 | 20.951 | 1.00 | 52.33 |
| ATOM | 529 | ND2 | ASN | 68 | 14.162 | 8.727 | 22.155 | 1.00 | 50.81 |
| ATOM | 530 | C | ASN | 68 | 11.107 | 9.511 | 19.920 | 1.00 | 40.11 |
| ATOM | 531 | O | ASN | 68 | 11.005 | 8.331 | 20.248 | 1.00 | 38.50 |
| ATOM | 532 | N | VAL | 69 | 11.031 | 9.922 | 18.657 | 1.00 | 38.66 |
| ATOM | 533 | CA | VAL | 69 | 10.794 | 9.008 | 17.539 | 1.00 | 39.78 |
| ATOM | 534 | CB | VAL | 69 | 10.383 | 9.806 | 16.277 | 1.00 | 38.68 |
| ATOM | 535 | CG1 | VAL | 69 | 9.997 | 8.867 | 15.165 | 1.00 | 39.74 |
| ATOM | 536 | CG2 | VAL | 69 | 9.221 | 10.733 | 16.600 | 1.00 | 37.97 |
| ATOM | 537 | C | VAL | 69 | 12.060 | 8.177 | 17.278 | 1.00 | 39.57 |
| ATOM | 538 | O | VAL | 69 | 13.156 | 8.741 | 17.193 | 1.00 | 40.97 |
| ATOM | 539 | N | PHE | 70 | 11.909 | 6.855 | 17.146 | 1.00 | 36.02 |
| ATOM | 540 | CA | PHE | 70 | 13.059 | 5.953 | 16.968 | 1.00 | 35.77 |
| ATOM | 541 | CB | PHE | 70 | 12.660 | 4.495 | 17.287 | 1.00 | 36.22 |
| ATOM | 542 | CG | PHE | 70 | 12.026 | 4.311 | 18.644 | 1.00 | 34.76 |
| ATOM | 543 | CD1 | PHE | 70 | 12.692 | 4.693 | 19.803 | 1.00 | 32.72 |
| ATOM | 544 | CD2 | PHE | 70 | 10.764 | 3.720 | 18.757 | 1.00 | 36.32 |
| ATOM | 545 | CE1 | PHE | 70 | 12.119 | 4.492 | 21.050 | 1.00 | 34.27 |
| ATOM | 546 | CE2 | PHE | 70 | 10.176 | 3.514 | 20.003 | 1.00 | 32.75 |
| ATOM | 547 | CZ | PHE | 70 | 10.857 | 3.901 | 21.157 | 1.00 | 34.27 |
| ATOM | 548 | C | PHE | 70 | 13.731 | 5.996 | 15.597 | 1.00 | 35.41 |
| ATOM | 549 | O | PHE | 70 | 14.947 | 5.811 | 15.474 | 1.00 | 35.74 |
| ATOM | 550 | N | ASP | 71 | 12.924 | 6.209 | 14.574 | 1.00 | 34.31 |
| ATOM | 551 | CA | ASP | 71 | 13.386 | 6.317 | 13.206 | 1.00 | 36.54 |
| ATOM | 552 | CE | ASP | 71 | 12.552 | 5.406 | 12.299 | 1.00 | 37.50 |
| ATOM | 553 | CG | ASP | 71 | 12.906 | 5.536 | 10.837 | 1.00 | 42.15 |
| ATOM | 554 | OD1 | ASP | 71 | 13.497 | 6.580 | 10.444 | 1.00 | 42.99 |
| ATOM | 555 | OD2 | ASP | 71 | 12.556 | 4.596 | 10.071 | 1.00 | 43.58 |
| ATOM | 556 | C | ASP | 71 | 13.156 | 7.797 | 12.855 | 1.00 | 36.95 |
| ATOM | 557 | O | ASP | 71 | 12.008 | 8.249 | 12.711 | 1.00 | 34.20 |
| ATOM | 558 | N | ARG | 72 | 14.261 | 8.530 | 12.743 | 1.00 | 37.72 |
| ATOM | 559 | CA | ARG | 72 | 14.260 | 9.957 | 12.453 | 1.00 | 41.91 |
| ATOM | 560 | CE | ARG | 72 | 15.714 | 10.444 | 12.340 | 1.00 | 44.20 |
| ATOM | 561 | CC | ARG | 72 | 15.89 | 711.931 | 12.101 | 1.00 | 50.61 |
| ATOM | 562 | CD | ARG | 72 | 17.379 | 12.304 | 12.148 | 1.00 | 54.03 |
| ATOM | 563 | NE | ARG | 72 | 17.935 | 12.179 | 13.495 | 1.00 | 58.08 |
| ATOM | 564 | CZ | ARG | 72 | 19.241 | 12.176 | 13.773 | 1.00 | 61.41 |
| ATOM | 565 | NH1 | ARG | 72 | 20.136 | 12.293 | 12.793 | 1.00 | 60.31 |
| ATOM | 566 | NH2 | ARG | 72 | 19.658 | 12.056 | 15.033 | 1.00 | 60.96 |
| ATOM | 567 | C | ARG | 72 | 13.447 | 10.317 | 11.205 | 1.00 | 42.06 |
| ATOM | 568 | O | ARG | 72 | 12.784 | 11.349 | 11.166 | 1.00 | 41.02 |
| ATOM | 569 | N | SER | 73 | 13.459 | 9.452 | 10.199 | 1.00 | 42.69 |
| ATOM | 570 | CA | SER | 73 | 12.700 | 9.719 | 8.991 | 1.00 | 41.82 |
| ATOM | 571 | CB | SER | 73 | 13.007 | 8.655 | 7.935 | 1.00 | 44.65 |
| ATOM | 572 | OG | SER | 73 | 12.665 | 7.354 | 8.392 | 1.00 | 47.00 |
| ATOM | 573 | C | SER | 73 | 11.190 | 9.779 | 9.233 | 1.00 | 42.72 |
| ATOM | 574 | O | SER | 73 | 10.449 | 10.336 | 8.417 | 1.00 | 42.14 |
| ATOM | 575 | N | MET | 74 | 10.730 | 9.199 | 10.338 | 1.00 | 41.95 |
| ATOM | 576 | CA | MET | 74 | 9.304 | 9.205 | 10.666 | 1.00 | 44.14 |
| ATOM | 577 | CB | MET | 74 | 8.932 | 7.959 | 11.479 | 1.00 | 44.72 |
| ATOM | 578 | CC | MET | 74 | 8.928 | 6.674 | 10.705 | 1.00 | 45.64 |
| ATOM | 579 | SD | MET | 74 | 7.787 | 6.778 | 9.304 | 1.00 | 49.27 |
| ATOM | 580 | CE | MET | 74 | 8.965 | 7.228 | 7.973 | 1.00 | 49.04 |
| ATOM | 581 | C | MET | 74 | 8.819 | 10.433 | 11.442 | 1.00 | 43.59 |
| ATOM | 582 | O | MET | 74 | 7.612 | 10.629 | 11.605 | 1.00 | 43.28 |
| ATOM | 583 | N | SER | 75 | 9.750 | 11.237 | 11.939 | 1.00 | 42.08 |
| ATOM | 584 | CA | SER | 75 | 9.381 | 12.406 | 12.715 | 1.00 | 42.19 |
| ATOM | 585 | CE | SER | 75 | 10.621 | 13.208 | 13.085 | 1.00 | 42.09 |
| ATOM | 586 | CC | SER | 75 | 10.281 | 14.280 | 13.941 | 1.00 | 46.20 |
| ATOM | 587 | C | SER | 75 | 8.376 | 13.301 | 11.991 | 1.00 | 42.07 |
| ATOM | 588 | O | SER | 75 | 7.364 | 13.698 | 12.574 | 1.00 | 40.55 |
| ATOM | 589 | N | PRO | 76 | 8.650 | 13.666 | 10.728 | 1.00 | 41.74 |
| ATOM | 590 | CD | PRO | 76 | 9.809 | 13.391 | 9.861 | 1.00 | 41.37 |
| ATOM | 591 | CA | PRO | 76 | 7.688 | 14.518 | 10.021 | 1.00 | 40.98 |
| ATOM | 592 | CB | PRO | 76 | 8.284 | 14.610 | 8.622 | 1.00 | 40.75 |
| ATOM | 593 | CC | PRO | 76 | 9.773 | 14.597 | 8.928 | 1.00 | 41.52 |
| ATOM | 594 | C | PRO | 76 | 6.283 | 13.904 | 10.027 | 1.00 | 40.31 |
| ATOM | 595 | O | PRO | 76 | 5.293 | 14.595 | 10.298 | 1.00 | 40.54 |
| ATOM | 596 | N | ALA | 77 | 6.207 | 12.607 | 9.737 | 1.00 | 38.47 |
| ATOM | 597 | CA | ALA | 77 | 4.926 | 11.897 | 9.711 | 1.00 | 39.23 |
| ATOM | 598 | CB | ALA | 77 | 5.130 | 10.451 | 9.209 | 1.00 | 39.21 |
| ATOM | 599 | C | ALA | 77 | 4.260 | 11.887 | 11.097 | 1.00 | 37.86 |
| ATOM | 600 | O | ALA | 77 | 3.042 | 11.977 | 11.204 | 1.00 | 37.27 |
| ATOM | 601 | N | VAL | 78 | 5.067 | 11.769 | 12.148 | 1.00 | 35.44 |
| ATOM | 602 | CA | VAL | 78 | 4.542 | 11.754 | 13.495 | 1.00 | 34.88 |
| ATOM | 603 | CE | VAL | 78 | 5.647 | 11.436 | 14.541 | 1.00 | 33.08 |
| ATOM | 604 | CG1 | VAL | 78 | 5.135 | 11.747 | 15.952 | 1.00 | 27.26 |
| ATOM | 605 | CG2 | VAL | 78 | 6.021 | 9.967 | 14.467 | 1.00 | 34.28 |
| ATOM | 606 | C | VAL | 78 | 3.942 | 13.107 | 13.812 | 1.00 | 36.71 |
| ATOM | 607 | O | VAL | 78 | 2.886 | 13.205 | 14.438 | 1.00 | 35.21 |
| ATOM | 608 | N | GLU | 79 | 4.641 | 14.153 | 13.388 | 1.00 | 38.95 |
| ATOM | 609 | CA | GLU | 79 | 4.193 | 15.507 | 13.628 | 1.00 | 41.23 |
| ATOM | 610 | CE | GLU | 79 | 5.248 | 16.517 | 13.183 | 1.00 | 42.75 |
| ATOM | 611 | CC | GLU | 79 | 4.774 | 17.950 | 13.309 | 1.00 | 49.69 |
| ATOM | 612 | CD | GLU | 79 | 5.928 | 18.925 | 13.461 | 1.00 | 53.09 |
| ATOM | 613 | OE1 | GLU | 79 | 6.899 | 18.828 | 12.664 | 1.00 | 56.30 |
| ATOM | 614 | OE2 | GLU | 79 | 5.858 | 19.784 | 14.370 | 1.00 | 50.54 |
| ATOM | 615 | C | GLU | 79 | 2.874 | 15.764 | 12.937 | 1.00 | 40.64 |
| ATOM | 616 | O | GLU | 79 | 1.996 | 16.380 | 13.517 | 1.00 | 43.12 |
| ATOM | 617 | N | LYS | 80 | 2.723 | 15.275 | 11.714 | 1.00 | 41.68 |
| ATOM | 618 | CA | LYS | 80 | 1.465 | 15.442 | 11.002 | 1.00 | 43.65 |
| ATOM | 619 | CE | LYS | 80 | 1.586 | 14.991 | 9.542 | 1.00 | 47.34 |
| ATOM | 620 | CC | LYS | 80 | 2.415 | 15.924 | 8.634 | 1.00 | 51.49 |
| ATOM | 621 | CD | LYS | 80 | 1.969 | 17.397 | 8.749 | 1.00 | 54.11 |
| ATOM | 622 | CE | LYS | 80 | 2.242 | 18.208 | 7.450 | 1.00 | 58.81 |
| ATOM | 623 | NZ | LYS | 80 | 3.659 | 18.188 | 6.930 | 1.00 | 59.99 |
| ATOM | 624 | C | LYS | 80 | 0.356 | 14.636 | 11.674 | 1.00 | 43.12 |
| ATOM | 625 | O | LYS | 80 | −0.800 | 15.077 | 11.748 | 1.00 | 43.15 |
| ATOM | 626 | N | ALA | 81 | 0.708 | 13.451 | 12.158 | 1.00 | 40.38 |
| ATOM | 627 | CA | ALA | 81 | −0.257 | 12.598 | 12.824 | 1.00 | 38.88 |
| ATOM | 628 | CE | ALA | 81 | 0.387 | 11.255 | 13.201 | 1.00 | 36.34 |
| ATOM | 629 | C | ALA | 81 | −0.785 | 13.302 | 14.072 | 1.00 | 39.56 |
| ATOM | 630 | O | ALA | 81 | −1.964 | 13.183 | 14.404 | 1.00 | 41.23 |
| ATOM | 631 | N | ILE | 82 | 0.074 | 14.045 | 14.766 | 1.00 | 39.16 |
| ATOM | 632 | CA | ILE | 82 | −0.380 | 14.720 | 15.971 | 1.00 | 38.15 |
| ATOM | 633 | CE | ILE | 82 | 0.780 | 15.267 | 16.803 | 1.00 | 35.54 |
| ATOM | 634 | CG2 | ILE | 82 | 0.241 | 16.060 | 17.965 | 1.00 | 33.27 |
| ATOM | 635 | CG1 | ILE | 82 | 1.626 | 14.111 | 17.351 | 1.00 | 35.88 |
| ATOM | 636 | CD1 | ILE | 82 | 2.878 | 14.562 | 18.122 | 1.00 | 36.02 |
| ATOM | 637 | C | ILE | 82 | −1.326 | 15.860 | 15.620 | 1.00 | 40.83 |
| ATOM | 638 | O | ILE | 82 | −2.373 | 16.019 | 16.231 | 1.00 | 39.39 |
| ATOM | 639 | N | MET | 83 | −0.965 | 16.660 | 14.633 | 1.00 | 42.37 |
| ATOM | 640 | CA | MET | 83 | −1.856 | 17.733 | 14.268 | 1.00 | 44.18 |
| ATOM | 641 | CE | MET | 83 | −1.238 | 18.589 | 13.164 | 1.00 | 47.58 |
| ATOM | 642 | CG | MET | 83 | 0.119 | 19.148 | 13.565 | 1.00 | 52.70 |
| ATOM | 643 | SD | MET | 83 | 0.871 | 20.375 | 12.470 | 1.00 | 58.98 |
| ATOM | 644 | CE | MET | 83 | 0.820 | 19.513 | 10.881 | 1.00 | 57.30 |
| ATOM | 645 | C | MET | 83 | −3.169 | 17.128 | 13.806 | 1.00 | 43.27 |
| ATOM | 646 | O | MET | 83 | −4.237 | 17.593 | 14.206 | 1.00 | 44.19 |
| ATOM | 647 | N | ALA | 84 | −3.081 | 16.044 | 13.034 | 1.00 | 41.67 |
| ATOM | 648 | CA | ALA | 84 | −4.260 | 15.376 | 12.456 | 1.00 | 40.91 |
| ATOM | 649 | CE | ALA | 84 | −3.821 | 14.343 | 11.427 | 1.00 | 39.91 |
| ATOM | 650 | C | ALA | 84 | −5.264 | 14.737 | 13.404 | 1.00 | 40.62 |
| ATOM | 651 | O | ALA | 84 | −6.427 | 14.590 | 13.059 | 1.00 | 40.16 |
| ATOM | 652 | N | SER | 85 | −4.823 | 14.338 | 14.586 | 1.00 | 40.70 |
| ATOM | 653 | CA | SER | 85 | −5.729 | 13.716 | 15.542 | 1.00 | 39.44 |
| ATOM | 654 | CB | SER | 85 | −4.956 | 13.233 | 16.774 | 1.00 | 37.78 |
| ATOM | 655 | OG | SER | 85 | −4.436 | 14.330 | 17.503 | 1.00 | 36.38 |
| ATOM | 656 | C | SER | 85 | −6.797 | 14.713 | 15.976 | 1.00 | 38.77 |
| ATOM | 657 | O | SER | 85 | −7.799 | 14.333 | 16.543 | 1.00 | 38.69 |
| ATOM | 658 | N | ASP | 86 | −6.550 | 15.993 | 15.724 | 1.00 | 40.30 |
| ATOM | 659 | CA | ASP | 86 | −7.477 | 17.073 | 16.070 | 1.00 | 39.72 |
| ATOM | 660 | CB | ASP | 86 | −8.750 | 16.964 | 15.244 | 1.00 | 40.60 |
| ATOM | 661 | CG | ASP | 86 | −9.690 | 18.137 | 15.462 | 1.00 | 43.77 |
| ATOM | 662 | OD1 | ASP | 86 | −9.259 | 19.302 | 15.305 | 1.00 | 45.04 |
| ATOM | 663 | OD2 | ASP | 86 | −10.872 | 17.896 | 15.782 | 1.00 | 45.78 |
| ATOM | 664 | C | ASP | 86 | −7.816 | 17.123 | 17.548 | 1.00 | 40.91 |
| ATOM | 665 | O | ASP | 86 | −8.980 | 17.131 | 17.948 | 1.00 | 40.62 |
| ATOM | 666 | N | LEU | 87 | −6.768 | 17.161 | 18.355 | 1.00 | 41.54 |
| ATOM | 667 | CA | LEU | 87 | −6.892 | 17.230 | 19.796 | 1.00 | 44.40 |
| ATOM | 668 | CB | LEU | 87 | −6.028 | 16.146 | 20.446 | 1.00 | 44.35 |
| ATOM | 669 | CG | LEU | 87 | −6.395 | 14.711 | 20.057 | 1.00 | 47.40 |
| ATOM | 670 | CD1 | LEU | 87 | −5.385 | 13.701 | 20.596 | 1.00 | 45.31 |
| ATOM | 671 | CD2 | LEU | 87 | −7.791 | 14.427 | 20.595 | 1.00 | 46.71 |
| ATOM | 672 | C | LEU | 87 | −6.452 | 18.614 | 20.273 | 1.00 | 46.06 |
| ATOM | 673 | O | LEU | 87 | −6.292 | 18.840 | 21.468 | 1.00 | 46.97 |
| ATOM | 674 | N | GLY | 88 | −6.245 | 19.527 | 19.324 | 1.00 | 48.12 |
| ATOM | 675 | CA | GLY | 88 | −5.841 | 20.883 | 19.654 | 1.00 | 47.94 |
| ATOM | 676 | C | GLY | 88 | −4.377 | 20.975 | 20.005 | 1.00 | 47.52 |
| ATOM | 677 | O | GLY | 88 | −3.891 | 22.030 | 20.404 | 1.00 | 47.24 |
| ATOM | 673 | N | LEU | 89 | −3.676 | 19.858 | 19.853 | 1.00 | 48.46 |
| ATOM | 679 | CA | LEU | 89 | −2.252 | 19.788 | 20.159 | 1.00 | 48.30 |
| ATOM | 680 | CB | LEU | 89 | −1.822 | 18.334 | 20.374 | 1.00 | 47.12 |
| ATOM | 681 | CC | LEU | 89 | −2.643 | 17.495 | 21.352 | 1.00 | 48.13 |
| ATOM | 682 | CD1 | LEU | 89 | −2.063 | 16.072 | 21.451 | 1.00 | 45.92 |
| ATOM | 683 | CD2 | LEU | 89 | −2.650 | 18.170 | 22.701 | 1.00 | 49.76 |
| ATOM | 684 | C | LEU | 89 | −1.400 | 20.404 | 19.043 | 1.00 | 49.93 |
| ATOM | 685 | O | LEU | 89 | −1.463 | 19.976 | 17.877 | 1.00 | 49.90 |
| ATOM | 686 | N | ASN | 90 | −0.611 | 21.410 | 19.423 | 1.00 | 49.86 |
| ATOM | 687 | CA | ASN | 90 | 0.292 | 22.120 | 18.523 | 1.00 | 50.18 |
| ATOM | 688 | CB | ASN | 90 | 0.305 | 23.625 | 18.852 | 1.00 | 51.76 |
| ATOM | 689 | CC | ASN | 90 | −1.056 | 24.282 | 18.689 | 1.00 | 51.96 |
| ATOM | 690 | OD1 | ASN | 90 | −1.630 | 24.289 | 17.604 | 1.00 | 52.89 |
| ATOM | 691 | OD2 | ASN | 90 | −1.576 | 24.845 | 19.774 | 1.00 | 55.10 |
| ATOM | 692 | C | ASN | 90 | 1.693 | 21.560 | 18.747 | 1.00 | 49.01 |
| ATOM | 693 | O | ASN | 90 | 2.419 | 22.030 | 19.617 | 1.00 | 47.33 |
| ATOM | 694 | N | PRO | 91 | 2.080 | 20.534 | 17.981 | 1.00 | 49.04 |
| ATOM | 695 | CD | PRO | 91 | 1.310 | 19.836 | 16.941 | 1.00 | 47.70 |
| ATOM | 696 | CA | PRO | 91 | 3.405 | 19.921 | 18.109 | 1.00 | 50.17 |
| ATOM | 697 | CB | PRO | 91 | 3.313 | 18.750 | 17.140 | 1.00 | 49.46 |
| ATOM | 698 | CG | PRO | 91 | 2.407 | 19.315 | 16.064 | 1.00 | 47.73 |
| ATOM | 699 | C | PRO | 91 | 4.519 | 20.901 | 17.734 | 1.00 | 52.02 |
| ATOM | 700 | O | PRO | 91 | 4.437 | 21.584 | 16.716 | 1.00 | 49.73 |
| ATOM | 701 | N | ASN | 92 | 5.551 | 20.978 | 18.569 | 1.00 | 55.38 |
| ATOM | 702 | CA | ASN | 92 | 6.677 | 21.876 | 18.316 | 1.00 | 56.64 |
| ATOM | 703 | CB | ASN | 92 | 6.907 | 22.779 | 19.527 | 1.00 | 57.88 |
| ATOM | 704 | CG | ASN | 92 | 5.868 | 23.895 | 19.624 | 1.00 | 61.72 |
| ATOM | 705 | OD1 | ASN | 92 | 5.687 | 24.507 | 20.679 | 1.00 | 63.08 |
| ATOM | 706 | OD2 | ASN | 92 | 5.195 | 24.178 | 18.509 | 1.00 | 61.74 |
| ATOM | 707 | C | ASN | 92 | 7.901 | 21.053 | 17.999 | 1.00 | 56.71 |
| ATOM | 708 | O | ASN | 92 | 8.540 | 20.511 | 18.889 | 1.00 | 56.47 |
| ATOM | 709 | N | SER | 93 | 8.212 | 20.957 | 16.711 | 1.00 | 58.68 |
| ATOM | 710 | CA | SER | 93 | 9.340 | 20.163 | 16.241 | 1.00 | 61.01 |
| ATOM | 711 | CB | SER | 93 | 9.026 | 19.614 | 14.846 | 1.00 | 60.28 |
| ATOM | 712 | CG | SER | 93 | 10.014 | 18.692 | 14.424 | 1.00 | 60.68 |
| ATOM | 713 | C | SER | 93 | 10.661 | 20.935 | 16.225 | 1.00 | 62.49 |
| ATOM | 714 | O | SER | 93 | 10.719 | 22.094 | 15.811 | 1.00 | 63.13 |
| ATOM | 715 | N | ALA | 94 | 11.724 | 20.281 | 16.677 | 1.00 | 63.76 |
| ATOM | 716 | CA | ALA | 94 | 13.019 | 20.930 | 16.716 | 1.00 | 65.86 |
| ATOM | 717 | CB | ALA | 94 | 12.971 | 22.123 | 17.669 | 1.00 | 65.68 |
| ATOM | 718 | C | ALA | 94 | 14.139 | 19.988 | 17.127 | 1.00 | 67.27 |
| ATOM | 719 | O | ALA | 94 | 14.181 | 19.508 | 18.260 | 1.00 | 67.78 |
| ATOM | 720 | N | GLY | 95 | 15.048 | 19.727 | 16.198 | 1.00 | 68.46 |
| ATOM | 721 | CA | GLY | 95 | 16.180 | 18.872 | 16.502 | 1.00 | 69.61 |
| ATOM | 722 | C | GLY | 95 | 15.883 | 17.489 | 17.045 | 1.00 | 69.64 |
| ATOM | 723 | O | GLY | 95 | 15.826 | 17.281 | 18.257 | 1.00 | 70.06 |
| ATOM | 724 | N | SER | 96 | 15.692 | 16.542 | 16.134 | 1.00 | 68.83 |
| ATOM | 725 | CA | SER | 96 | 15.434 | 15.151 | 16.487 | 1.00 | 68.81 |
| ATOM | 726 | CB | SER | 96 | 16.734 | 14.514 | 16.984 | 1.00 | 67.99 |
| ATOM | 727 | CG | SER | 96 | 17.708 | 14.490 | 15.954 | 1.00 | 69.91 |
| ATOM | 728 | C | SER | 96 | 14.320 | 14.901 | 17.514 | 1.00 | 68.02 |
| ATOM | 729 | O | SER | 96 | 13.902 | 13.759 | 17.726 | 1.00 | 69.37 |
| ATOM | 730 | N | ASP | 97 | 13.819 | 15.959 | 18.135 | 1.00 | 65.66 |
| ATOM | 731 | CA | ASP | 97 | 12.792 | 15.801 | 19.149 | 1.00 | 61.46 |
| ATOM | 732 | CB | ASP | 97 | 13.427 | 16.128 | 20.508 | 1.00 | 64.43 |
| ATOM | 733 | CC | ASP | 97 | 12.454 | 16.730 | 21.498 | 1.00 | 68.00 |
| ATOM | 734 | OD1 | ASP | 97 | 11.686 | 17.632 | 21.096 | 1.00 | 70.75 |
| ATOM | 735 | OD2 | ASP | 97 | 12.480 | 16.339 | 22.690 | 1.00 | 69.61 |
| ATOM | 736 | C | ASP | 97 | 11.563 | 16.657 | 18.874 | 1.00 | 57.75 |
| ATOM | 737 | O | ASP | 97 | 11.662 | 17.760 | 18.342 | 1.00 | 56.47 |
| ATOM | 738 | N | ILE | 98 | 10.399 | 16.116 | 19.220 | 1.00 | 54.15 |
| ATOM | 739 | CA | ILE | 98 | 9.129 | 16.810 | 19.063 | 1.00 | 48.95 |
| ATOM | 740 | CS | ILE | 98 | 8.069 | 15.928 | 18.372 | 1.00 | 48.45 |
| ATOM | 741 | CG2 | ILE | 98 | 6.724 | 16.639 | 18.364 | 1.00 | 46.97 |
| ATOM | 742 | CG1 | ILE | 98 | 8.519 | 15.571 | 16.955 | 1.00 | 47.46 |
| ATOM | 743 | CD1 | ILE | 98 | 7.566 | 14.651 | 16.22 | 91.00 | 45.26 |
| ATOM | 744 | C | ILE | 98 | 8.633 | 17.073 | 20.469 | 1.00 | 48.06 |
| ATOM | 745 | O | ILE | 98 | 8.669 | 16.187 | 21.310 | 1.00 | 47.48 |
| ATOM | 746 | N | ARG | 99 | 8.202 | 18.291 | 20.752 | 1.00 | 46.99 |
| ATOM | 747 | CA | ARG | 99 | 7.660 | 18.556 | 22.069 | 1.00 | 47.18 |
| ATOM | 748 | CB | ARG | 99 | 8.347 | 19.738 | 22.730 | 1.00 | 48.61 |
| ATOM | 749 | CC | ARG | 99 | 9.780 | 19.436 | 23.088 | 1.00 | 52.92 |
| ATOM | 750 | CD | ARG | 99 | 10.435 | 20.597 | 23.774 | 1.00 | 53.16 |
| ATOM | 751 | NE | ARG | 99 | 11.875 | 20.550 | 23.580 | 1.00 | 59.38 |
| ATOM | 752 | CZ | ARG | 99 | 12.656 | 19.533 | 23.930 | 1.00 | 62.25 |
| ATOM | 753 | NH1 | ARG | 99 | 12.137 | 18.453 | 24.507 | 1.00 | 63.64 |
| ATOM | 754 | NH2 | ARG | 99 | 13.963 | 19.594 | 23.687 | 1.00 | 64.01 |
| ATOM | 755 | C | ARG | 99 | 6.182 | 18.816 | 21.892 | 1.00 | 45.57 |
| ATOM | 756 | O | ARG | 99 | 5.748 | 19.325 | 20.853 | 1.00 | 45.31 |
| ATOM | 757 | N | VAL | 100 | 5.408 | 18.451 | 22.901 | 1.00 | 43.60 |
| ATOM | 758 | CA | VAL | 100 | 3.967 | 18.601 | 22.834 | 1.00 | 42.41 |
| ATOM | 759 | CB | VAL | 100 | 3.295 | 17.210 | 22.698 | 1.00 | 42.94 |
| ATOM | 760 | CG1 | VAL | 100 | 1.774 | 17.350 | 22.702 | 1.00 | 44.36 |
| ATOM | 761 | CG2 | VAL | 100 | 3.792 | 16.508 | 21.427 | 1.00 | 41.91 |
| ATOM | 762 | C | VAL | 100 | 3.417 | 19.310 | 24.065 | 1.00 | 41.58 |
| ATOM | 763 | O | VAL | 100 | 3.209 | 18.698 | 25.110 | 1.00 | 40.47 |
| ATOM | 764 | N | PRO | 101 | 3.236 | 20.632 | 23.977 | 1.00 | 41.65 |
| ATOM | 765 | CD | PRO | 101 | 3.568 | 21.570 | 22.889 | 1.00 | 41.07 |
| ATOM | 766 | CA | PRO | 101 | 2.696 | 21.361 | 25.122 | 1.00 | 39.69 |
| ATOM | 767 | CB | PRO | 101 | 2.830 | 22.809 | 24.675 | 1.00 | 40.75 |
| ATOM | 768 | CO | PRO | 101 | 2.608 | 22.690 | 23.169 | 1.00 | 40.97 |
| ATOM | 769 | C | PRO | 101 | 1.243 | 20.906 | 25.263 | 1.00 | 38.36 |
| ATOM | 770 | O | PRO | 101 | 0.566 | 20.638 | 24.273 | 1.00 | 36.38 |
| ATOM | 771 | N | LEU | 102 | 0.766 | 20.816 | 26.491 | 1.00 | 37.40 |
| ATOM | 772 | CA | LEU | 102 | −0.597 | 20.349 | 26.728 | 1.00 | 36.57 |
| ATOM | 773 | CS | LEU | 102 | −0.536 | 19.081 | 27.605 | 1.00 | 36.65 |
| ATOM | 774 | CG | LEU | 102 | 0.247 | 17.915 | 26.968 | 1.00 | 34.92 |
| ATOM | 775 | CD1 | LEU | 102 | 0.509 | 16.837 | 27.969 | 1.00 | 34.58 |
| ATOM | 776 | CD2 | LEU | 102 | −0.518 | 17.391 | 25.785 | 1.00 | 31.23 |
| ATOM | 777 | C | LEU | 102 | −1.536 | 21.375 | 27.364 | 1.00 | 35.46 |
| ATOM | 778 | O | LEU | 102 | −1.148 | 22.129 | 28.247 | 1.00 | 32.96 |
| ATOM | 779 | N | PRO | 103 | −2.809 | 21.379 | 26.945 | 1.00 | 37.48 |
| ATOM | 780 | CD | PRO | 103 | −3.456 | 20.510 | 25.948 | 1.00 | 38.55 |
| ATOM | 781 | CA | PRO | 103 | −3.797 | 22.311 | 27.485 | 1.00 | 39.19 |
| ATOM | 782 | CS | PRO | 103 | −5.015 | 22.043 | 26.599 | 1.00 | 38.49 |
| ATOM | 783 | CC | PRO | 103 | −4.921 | 20.592 | 26.384 | 1.00 | 36.60 |
| ATOM | 784 | C | PRO | 103 | −4.055 | 21.999 | 28.960 | 1.00 | 41.74 |
| ATOM | 785 | O | PRO | 103 | −3.955 | 20.850 | 29.388 | 1.00 | 41.12 |
| ATOM | 786 | N | PRO | 104 | −4.386 | 23.018 | 29.763 | 1.00 | 45.20 |
| ATOM | 787 | CD | PRO | 104 | −4.533 | 24.458 | 29.473 | 1.00 | 46.97 |
| ATOM | 788 | CA | PRO | 104 | −4.643 | 22.772 | 31.184 | 1.00 | 44.68 |
| ATOM | 789 | CS | PRO | 104 | −4.783 | 24.177 | 31.745 | 1.00 | 44.21 |
| ATOM | 790 | CC | PRO | 104 | −5.461 | 24.893 | 30.601 | 1.00 | 47.15 |
| ATOM | 791 | C | PRO | 104 | −5.911 | 21.957 | 31.390 | 1.00 | 45.36 |
| ATOM | 792 | O | PRO | 104 | −6.735 | 21.812 | 30.480 | 1.00 | 46.09 |
| ATOM | 793 | N | LEU | 105 | −6.056 | 21.400 | 32.580 | 1.00 | 44.16 |
| ATOM | 794 | CA | LEU | 105 | −7.260 | 20.654 | 32.889 | 1.00 | 43.98 |
| ATOM | 795 | CE | LEU | 105 | −7.208 | 20.155 | 34.327 | 1.00 | 42.52 |
| ATOM | 796 | CC | LEU | 105 | −6.071 | 19.208 | 34.668 | 1.00 | 41.22 |
| ATOM | 797 | CD1 | LEU | 105 | −6.052 | 18.923 | 36.174 | 1.00 | 40.33 |
| ATOM | 798 | CD2 | LEU | 105 | −6.263 | 17.944 | 33.861 | 1.00 | 39.80 |
| ATOM | 799 | C | LEU | 105 | −8.398 | 21.655 | 32.739 | 1.00 | 43.38 |
| ATOM | 800 | O | LEU | 105 | −8.199 | 22.860 | 32.924 | 1.00 | 44.06 |
| ATOM | 801 | N | THR | 106 | −9.588 | 21.175 | 32.412 | 1.00 | 41.61 |
| ATOM | 802 | CA | THR | 106 | −10.709 | 22.087 | 32.255 | 1.00 | 42.62 |
| ATOM | 803 | CE | THR | 106 | −11.943 | 21.367 | 31.673 | 1.00 | 40.92 |
| ATOM | 804 | OG1 | THR | 106 | −12.315 | 20.274 | 32.518 | 1.00 | 38.13 |
| ATOM | 805 | OG2 | THR | 106 | −11.626 | 20.847 | 30.278 | 1.00 | 41.50 |
| ATOM | 806 | C | THR | 106 | −11.095 | 22.774 | 33.566 | 1.00 | 43.42 |
| ATOM | 807 | O | THR | 106 | −10.777 | 22.301 | 34.651 | 1.00 | 40.88 |
| ATOM | 808 | N | GLU | 107 | −11.777 | 23.903 | 33.439 | 1.00 | 46.29 |
| ATOM | 809 | CA | GLU | 107 | −12.225 | 24.682 | 34.578 | 1.00 | 50.04 |
| ATOM | 810 | CE | GLU | 107 | −13.100 | 25.840 | 34.094 | 1.00 | 55.08 |
| ATOM | 811 | CG | GLU | 107 | −12.939 | 27.144 | 34.864 | 1.00 | 61.74 |
| ATOM | 812 | CO | GLU | 107 | −11.783 | 27.997 | 34.337 | 1.00 | 65.24 |
| ATOM | 813 | OE1 | GLU | 107 | −11.844 | 28.413 | 33.158 | 1.00 | 66.53 |
| ATOM | 814 | OE2 | GLU | 107 | −10.820 | 28.257 | 35.096 | 1.00 | 68.36 |
| ATOM | 815 | C | GLU | 107 | −13.064 | 23.760 | 35.464 | 1.00 | 49.91 |
| ATOM | 816 | O | GLU | 107 | −12.860 | 23.673 | 36.677 | 1.00 | 49.39 |
| ATOM | 817 | N | GLU | 108 | −14.004 | 23.067 | 34.835 | 1.00 | 49.83 |
| ATOM | 818 | CA | GLU | 108 | −14.894 | 22.146 | 35.526 | 1.00 | 50.79 |
| ATOM | 819 | CE | GLU | 108 | −15.887 | 21.532 | 34.523 | 1.00 | 52.77 |
| ATOM | 820 | CG | GLU | 108 | −17.030 | 20.740 | 35.160 | 1.00 | 57.30 |
| ATOM | 821 | CD | GLU | 108 | −17.865 | 21.574 | 36.143 | 1.00 | 60.57 |
| ATOM | 822 | OE1 | GLU | 108 | −18.370 | 22.655 | 35.757 | 1.00 | 61.21 |
| ATOM | 823 | OE2 | GLU | 108 | −18.018 | 21.141 | 37.310 | 1.00 | 63.46 |
| ATOM | 824 | C | GLU | 108 | −14.126 | 21.043 | 36.262 | 1.00 | 49.48 |
| ATOM | 825 | O | GLU | 108 | −14.509 | 20.636 | 37.369 | 1.00 | 48.71 |
| ATOM | 826 | N | ARG | 109 | −13.042 | 20.566 | 35.656 | 1.00 | 47.01 |
| ATOM | 827 | CA | ARG | 109 | −12.224 | 19.528 | 36.278 | 1.00 | 45.40 |
| ATOM | 828 | CE | ARG | 109 | −11.211 | 18.982 | 35.270 | 1.00 | 45.36 |
| ATOM | 829 | CG | ARG | 109 | −10.308 | 17.876 | 35.792 | 1.00 | 46.17 |
| ATOM | 830 | CD | ARG | 109 | −11.096 | 16.681 | 36.378 | 1.00 | 48.74 |
| ATOM | 831 | NE | ARG | 109 | −10.190 | 15.611 | 36.810 | 1.00 | 51.07 |
| ATOM | 832 | CZ | ARG | 109 | −10.550 | 14.537 | 37.515 | 1.00 | 50.91 |
| ATOM | 833 | NH1 | ARG | 109 | −11.810 | 14.363 | 37.886 | 1.00 | 50.39 |
| ATOM | 834 | NH2 | ARG | 109 | −9.640 | 13.643 | 37.868 | 1.00 | 51.02 |
| ATOM | 835 | C | ARG | 109 | −11.497 | 20.076 | 37.508 | 1.00 | 44.07 |
| ATOM | 836 | O | ARG | 109 | −11.536 | 19.484 | 38.582 | 1.00 | 41.18 |
| ATOM | 837 | N | ARG | 110 | −10.836 | 21.214 | 37.357 | 1.00 | 44.70 |
| ATOM | 838 | CA | ARG | 110 | −10.128 | 21.790 | 38.484 | 1.00 | 45.62 |
| ATOM | 839 | CE | ARG | 110 | −9.532 | 23.145 | 38.109 | 1.00 | 49.45 |
| ATOM | 840 | CC | ARG | 110 | −8.769 | 23.823 | 39.239 | 1.00 | 55.43 |
| ATOM | 841 | CD | ARG | 110 | −7.813 | 24.933 | 38.761 | 1.00 | 58.14 |
| ATOM | 842 | NE | ARG | 110 | −8.383 | 25.798 | 37.729 | 1.00 | 59.04 |
| ATOM | 843 | CZ | ARG | 110 | −8.436 | 25.487 | 36.435 | 1.00 | 61.75 |
| ATOM | 844 | NH1 | ARG | 110 | −7.945 | 24.328 | 36.006 | 1.00 | 61.67 |
| ATOM | 845 | NH2 | ARG | 110 | −8.993 | 26.331 | 35.572 | 1.00 | 61.81 |
| ATOM | 846 | C | ARG | 110 | −11.072 | 21.920 | 39.667 | 1.00 | 45.36 |
| ATOM | 847 | O | ARG | 110 | −10.697 | 21.602 | 40.804 | 1.00 | 44.82 |
| ATOM | 848 | N | LYS | 111 | −12.300 | 22.363 | 39.404 | 1.00 | 44.64 |
| ATOM | 849 | CA | LYS | 111 | −13.283 | 22.500 | 40.476 | 1.00 | 46.68 |
| ATOM | 850 | CE | LYS | 111 | −14.549 | 23.225 | 39.997 | 1.00 | 48.26 |
| ATOM | 851 | CG | LYS | 111 | −14.313 | 24.695 | 39.665 | 1.00 | 53.39 |
| ATOM | 852 | CD | LYS | 111 | −15.621 | 25.451 | 39.447 | 1.00 | 56.17 |
| ATOM | 853 | CE | LYS | 111 | −15.382 | 26.917 | 39.046 | 1.00 | 58.69 |
| ATOM | 854 | NE | LYS | 111 | −14.714 | 27.105 | 37.709 | 1.00 | 56.88 |
| ATOM | 855 | C | LYS | 111 | −13.640 | 21.138 | 41.067 | 1.00 | 44.82 |
| ATOM | 856 | O | LYS | 111 | −13.726 | 21.010 | 42.276 | 1.00 | 44.23 |
| ATOM | 857 | N | ASP | 112 | −13.841 | 20.125 | 40.231 | 1.00 | 45.41 |
| ATOM | 858 | CA | ASP | 112 | −14.141 | 18.793 | 40.762 | 1.00 | 47.37 |
| ATOM | 859 | CB | ASP | 112 | −14.362 | 17.766 | 39.634 | 1.00 | 49.39 |
| ATOM | 860 | CG | ASP | 112 | −15.388 | 18.222 | 38.599 | 1.00 | 56.37 |
| ATOM | 861 | OD1 | ASP | 112 | −16.549 | 18.548 | 38.976 | 1.00 | 58.22 |
| ATOM | 862 | OD2 | ASP | 112 | −15.030 | 18.245 | 37.388 | 1.00 | 58.89 |
| ATOM | 863 | C | ASP | 112 | −12.966 | 18.304 | 41.657 | 1.00 | 46.29 |
| ATOM | 864 | O | ASP | 112 | −13.179 | 17.652 | 42.672 | 1.00 | 47.08 |
| ATOM | 865 | N | LEU | 113 | −11.728 | 18.622 | 41.288 | 1.00 | 42.72 |
| ATOM | 866 | CA | LEU | 113 | −10.582 | 18.181 | 42.084 | 1.00 | 40.11 |
| ATOM | 867 | CB | LEU | 113 | −9.323 | 18.172 | 41.222 | 1.00 | 37.15 |
| ATOM | 868 | CG | LEU | 113 | −9.390 | 17.125 | 40.111 | 1.00 | 36.21 |
| ATOM | 869 | CD1 | LEU | 113 | −8.274 | 17.320 | 39.106 | 1.00 | 36.61 |
| ATOM | 870 | CD2 | LEU | 113 | −9.341 | 15.744 | 40.742 | 1.00 | 37.64 |
| ATOM | 871 | C | LEU | 113 | −10.370 | 19.027 | 43.340 | 1.00 | 39.82 |
| ATOM | 872 | O | LEU | 113 | −9.973 | 18.509 | 44.400 | 1.00 | 37.85 |
| ATOM | 873 | N | THR | 114 | −10.638 | 20.325 | 43.228 | 1.00 | 38.24 |
| ATOM | 874 | CA | THR | 114 | −10.479 | 21.206 | 44.375 | 1.00 | 37.09 |
| ATOM | 875 | CB | THR | 114 | −10.828 | 22.666 | 44.019 | 1.00 | 36.57 |
| ATOM | 876 | OG1 | THR | 114 | −9.924 | 23.153 | 43.017 | 1.00 | 33.29 |
| ATOM | 877 | OG2 | THR | 114 | −10.723 | 23.547 | 45.252 | 1.00 | 37.73 |
| ATOM | 878 | C | THR | 114 | −11.459 | 20.708 | 45.417 | 1.00 | 39.56 |
| ATOM | 879 | O | THR | 114 | −11.163 | 20.652 | 46.611 | 1.00 | 37.29 |
| ATOM | 880 | N | LYS | 115 | −12.638 | 20.319 | 44.946 | 1.00 | 40.54 |
| ATOM | 881 | CA | LYS | 115 | −13.661 | 19.842 | 45.848 | 1.00 | 41.37 |
| ATOM | 882 | CB | LYS | 115 | −14.974 | 19.666 | 45.083 | 1.00 | 45.84 |
| ATOM | 883 | CC | LYS | 115 | −16.101 | 19.059 | 45.880 | 1.00 | 49.09 |
| ATOM | 884 | CD | LYS | 115 | −17.418 | 19.248 | 45.151 | 1.00 | 53.42 |
| ATOM | 885 | CE | LYS | 115 | −18.522 | 18.381 | 45.760 | 1.00 | 56.03 |
| ATOM | 886 | NE | LYS | 115 | −18.314 | 16.917 | 45.456 | 1.00 | 57.60 |
| ATOM | 887 | C | LYS | 115 | −13.242 | 18.561 | 46.542 | 1.00 | 38.50 |
| ATOM | 888 | O | LYS | 115 | −13.495 | 18.382 | 47.720 | 1.00 | 37.67 |
| ATOM | 889 | N | ILE | 116 | −12.560 | 17.685 | 45.823 | 1.00 | 38.50 |
| ATOM | 890 | CA | ILE | 116 | −12.133 | 16.434 | 46.419 | 1.00 | 37.98 |
| ATOM | 891 | CB | ILE | 116 | −11.617 | 15.452 | 45.374 | 1.00 | 39.26 |
| ATOM | 892 | CG2 | ILE | 116 | −11.367 | 14.104 | 46.027 | 1.00 | 39.71 |
| ATOM | 893 | CG1 | ILE | 116 | −12.640 | 15.297 | 44.257 | 1.00 | 39.44 |
| ATOM | 894 | CD1 | ILE | 116 | −13.980 | 14.876 | 44.742 | 1.00 | 42.71 |
| ATOM | 895 | C | ILE | 116 | −11.050 | 16.602 | 47.474 | 1.00 | 37.87 |
| ATOM | 896 | O | ILE | 116 | −11.127 | 15.979 | 48.537 | 1.00 | 38.93 |
| ATOM | 897 | N | VAL | 117 | −10.041 | 17.425 | 47.209 | 1.00 | 35.17 |
| ATOM | 898 | CA | VAL | 117 | −8.996 | 17.584 | 48.220 | 1.00 | 35.04 |
| ATOM | 899 | CB | VAL | 117 | −7.764 | 18.330 | 47.666 | 1.00 | 32.48 |
| ATOM | 900 | CG1 | VAL | 117 | −7.220 | 17.566 | 46.481 | 1.00 | 33.10 |
| ATOM | 901 | CG2 | VAL | 117 | −8.123 | 19.714 | 47.248 | 1.00 | 32.62 |
| ATOM | 902 | C | VAL | 117 | −9.560 | 18.268 | 49.468 | 1.00 | 33.43 |
| ATOM | 903 | O | VAL | 117 | −9.215 | 17.897 | 50.596 | 1.00 | 33.84 |
| ATOM | 904 | N | ARG | 118 | −10.442 | 19.246 | 49.274 | 1.00 | 33.17 |
| ATOM | 905 | CA | ARG | 118 | −11.075 | 19.918 | 50.419 | 1.00 | 33.39 |
| ATOM | 906 | CB | ARG | 118 | −11.982 | 21.060 | 49.961 | 1.00 | 29.92 |
| ATOM | 907 | CC | ARG | 118 | −11.192 | 22.288 | 49.649 | 1.00 | 32.89 |
| ATOM | 908 | CD | ARG | 118 | −12.029 | 23.334 | 49.036 | 1.00 | 37.88 |
| ATOM | 909 | NE | ARG | 118 | −11.219 | 24.462 | 48.622 | 1.00 | 41.77 |
| ATOM | 910 | CZ | ARG | 118 | −11.695 | 25.532 | 48.003 | 1.00 | 46.19 |
| ATOM | 911 | NH1 | ARG | 118 | −12.987 | 25.626 | 47.718 | 1.00 | 47.00 |
| ATOM | 912 | NH2 | ARG | 118 | −10.871 | 26.517 | 47.679 | 1.00 | 49.71 |
| ATOM | 913 | C | ARG | 118 | −11.859 | 18.945 | 51.297 | 1.00 | 32.57 |
| ATOM | 914 | O | ARG | 118 | −11.806 | 19.046 | 52.515 | 1.00 | 36.89 |
| ATOM | 915 | N | GLY | 119 | −12.570 | 18.008 | 50.677 | 1.00 | 30.46 |
| ATOM | 916 | CA | GLY | 119 | −13.322 | 17.018 | 51.423 | 1.00 | 29.23 |
| ATOM | 917 | C | GLY | 119 | −12.407 | 16.082 | 52.202 | 1.00 | 29.62 |
| ATOM | 918 | O | GLY | 119 | −12.702 | 15.722 | 53.335 | 1.00 | 30.06 |
| ATOM | 919 | N | GLU | 120 | −11.294 | 15.692 | 51.593 | 1.00 | 30.52 |
| ATOM | 920 | CA | GLU | 120 | −10.321 | 14.827 | 52.241 | 1.00 | 31.96 |
| ATOM | 921 | CB | GLU | 120 | −9.281 | 14.349 | 51.222 | 1.00 | 34.72 |
| ATOM | 922 | CC | GLU | 120 | −9.912 | 13.483 | 50.152 | 1.00 | 39.64 |
| ATOM | 923 | CD | GLU | 120 | −8.962 | 13.047 | 49.049 | 1.00 | 42.12 |
| ATOM | 924 | OE1 | GLU | 120 | −9.414 | 12.270 | 48.184 | 1.00 | 44.99 |
| ATOM | 925 | OE2 | GLU | 120 | −7.780 | 13.461 | 49.030 | 1.00 | 43.53 |
| ATOM | 926 | C | GLU | 120 | −9.649 | 15.592 | 53.368 | 1.00 | 32.43 |
| ATOM | 927 | O | GLU | 120 | −9.499 | 15.063 | 54.462 | 1.00 | 35.07 |
| ATOM | 928 | N | ALA | 121 | −9.260 | 16.845 | 53.124 | 1.00 | 32.32 |
| ATOM | 929 | CA | ALA | 121 | −8.631 | 17.616 | 54.186 | 1.00 | 31.62 |
| ATOM | 930 | CB | ALA | 121 | −8.336 | 19.035 | 53.720 | 1.00 | 28.12 |
| ATOM | 931 | C | ALA | 121 | −9.624 | 17.634 | 55.344 | 1.00 | 34.83 |
| ATOM | 932 | O | ALA | 121 | −9.293 | 17.273 | 56.475 | 1.00 | 36.01 |
| ATOM | 933 | N | GLU | 122 | −10.858 | 18.018 | 55.026 | 1.00 | 35.31 |
| ATOM | 934 | CA | GLU | 122 | −11.944 | 18.115 | 55.992 | 1.00 | 36.50 |
| ATOM | 935 | CB | GLU | 122 | −13.259 | 18.348 | 55.242 | 1.00 | 39.77 |
| ATOM | 936 | CC | GLU | 122 | −14.201 | 19.288 | 55.924 | 1.00 | 39.83 |
| ATOM | 937 | CD | GLU | 122 | −14.446 | 18.903 | 57.343 | 1.00 | 40.58 |
| ATOM | 938 | OE1 | GLU | 122 | −14.903 | 17.768 | 57.553 | 1.00 | 44.91 |
| ATOM | 939 | OE2 | GLU | 122 | −14.184 | 19.728 | 58.250 | 1.00 | 41.71 |
| ATOM | 940 | C | GLU | 122 | −12.028 | 16.836 | 56.808 | 1.00 | 36.15 |
| ATOM | 941 | O | GLU | 122 | −12.081 | 16.875 | 58.037 | 1.00 | 34.04 |
| ATOM | 942 | N | GLN | 123 | −12.053 | 15.698 | 56.115 | 1.00 | 36.64 |
| ATOM | 943 | CA | GLN | 123 | −12.099 | 14.413 | 56.791 | 1.00 | 38.70 |
| ATOM | 944 | CB | GLN | 123 | −12.070 | 13.295 | 55.751 | 1.00 | 40.64 |
| ATOM | 945 | CC | GLN | 123 | −11.999 | 11.888 | 56.331 | 1.00 | 45.87 |
| ATOM | 946 | CD | GLN | 123 | −10.575 | 11.398 | 56.525 | 1.00 | 50.88 |
| ATOM | 947 | OE1 | GLN | 123 | −9.774 | 11.378 | 55.579 | 1.00 | 54.39 |
| ATOM | 948 | NE2 | GLN | 123 | −10.254 | 10.983 | 57.739 | 1.00 | 50.01 |
| ATOM | 949 | C | GLN | 123 | −10.910 | 14.277 | 57.760 | 1.00 | 37.35 |
| ATOM | 950 | O | GLN | 123 | −11.070 | 13.855 | 58.900 | 1.00 | 37.65 |
| ATOM | 951 | N | ALA | 124 | −9.721 | 14.652 | 57.296 | 1.00 | 35.43 |
| ATOM | 952 | CA | ALA | 124 | −8.522 | 14.576 | 58.123 | 1.00 | 34.84 |
| ATOM | 953 | CB | ALA | 124 | −7.252 | 15.024 | 57.307 | 1.00 | 33.06 |
| ATOM | 954 | C | ALA | 124 | −8.670 | 15.431 | 59.384 | 1.00 | 32.69 |
| ATOM | 955 | O | ALA | 124 | −8.236 | 15.024 | 60.470 | 1.00 | 31.59 |
| ATOM | 956 | N | ARG | 125 | −9.266 | 16.612 | 59.237 | 1.00 | 29.13 |
| ATOM | 957 | CA | ARG | 125 | −9.464 | 17.488 | 60.377 | 1.00 | 30.40 |
| ATOM | 958 | CB | ARG | 125 | −9.883 | 18.901 | 59.943 | 1.00 | 33.05 |
| ATOM | 959 | CC | ARG | 125 | −8.734 | 19.821 | 59.545 | 1.00 | 36.01 |
| ATOM | 960 | CD | ARG | 125 | −9.244 | 21.249 | 59.398 | 1.00 | 37.50 |
| ATOM | 961 | NE | ARG | 125 | −10.102 | 21.309 | 58.240 | 1.00 | 41.70 |
| ATOM | 962 | CZ | ARG | 125 | −9.683 | 21.653 | 57.037 | 1.00 | 39.98 |
| ATOM | 963 | NE1 | ARG | 125 | −8.415 | 21.979 | 56.856 | 1.00 | 42.73 |
| ATOM | 964 | NH2 | ARG | 125 | −10.502 | 21.568 | 56.007 | 1.00 | 37.33 |
| ATOM | 965 | C | ARG | 125 | −10.468 | 16.943 | 61.386 | 1.00 | 30.54 |
| ATOM | 966 | O | ARG | 125 | −10.296 | 17.144 | 62.580 | 1.00 | 31.78 |
| ATOM | 967 | N | VAL | 126 | −11.511 | 16.262 | 60.923 | 1.00 | 30.13 |
| ATOM | 968 | CA | VAL | 126 | −12.474 | 15.683 | 61.847 | 1.00 | 29.18 |
| ATOM | 969 | CB | VAL | 126 | −13.762 | 15.137 | 61.107 | 1.00 | 30.93 |
| ATOM | 970 | CG1 | VAL | 126 | −14.721 | 14.517 | 62.104 | 1.00 | 26.39 |
| ATOM | 971 | CG2 | VAL | 126 | −14.509 | 16.292 | 60.354 | 1.00 | 28.70 |
| ATOM | 972 | C | VAL | 126 | −11.754 | 14.531 | 62.583 | 1.00 | 30.04 |
| ATOM | 973 | O | VAL | 126 | −11.820 | 14.422 | 63.816 | 1.00 | 31.66 |
| ATOM | 974 | N | ALA | 127 | −11.031 | 13.698 | 61.849 | 1.00 | 28.96 |
| ATOM | 975 | CA | ALA | 127 | −10.337 | 12.583 | 62.492 | 1.00 | 30.05 |
| ATOM | 976 | CB | ALA | 127 | −9.574 | 11.753 | 61.434 | 1.00 | 29.30 |
| ATOM | 977 | C | ALA | 127 | −9.374 | 13.067 | 63.589 | 1.00 | 29.90 |
| ATOM | 978 | O | ALA | 127 | −9.308 | 12.493 | 64.673 | 1.00 | 31.73 |
| ATOM | 979 | N | VAL | 128 | −8.643 | 14.137 | 63.312 | 1.00 | 31.82 |
| ATOM | 980 | CA | VAL | 128 | −7.686 | 14.694 | 64.264 | 1.00 | 31.72 |
| ATOM | 981 | CB | VAL | 128 | −6.844 | 15.819 | 63.581 | 1.00 | 33.25 |
| ATOM | 982 | CG1 | VAL | 128 | −5.997 | 16.559 | 64.626 | 1.00 | 35.15 |
| ATOM | 983 | CG2 | VAL | 128 | −5.936 | 15.195 | 62.504 | 1.00 | 28.98 |
| ATOM | 984 | C | VAL | 128 | −8.346 | 15.229 | 65.546 | 1.00 | 34.61 |
| ATOM | 985 | O | VAL | 128 | −7.795 | 15.063 | 66.644 | 1.00 | 34.11 |
| ATOM | 986 | N | ARG | 129 | −9.514 | 15.863 | 65.396 | 1.00 | 34.97 |
| ATOM | 987 | CA | ARG | 129 | −10.285 | 16.415 | 66.518 | 1.00 | 36.61 |
| ATOM | 988 | CB | ARG | 129 | −11.416 | 17.335 | 66.048 | 1.00 | 39.01 |
| ATOM | 989 | CG | ARG | 129 | −11.084 | 18.404 | 65.030 | 1.00 | 42.15 |
| ATOM | 990 | CD | ARG | 129 | −12.401 | 18.970 | 64.452 | 1.00 | 43.95 |
| ATOM | 991 | NE | ARG | 129 | −12.193 | 19.869 | 63.321 | 1.00 | 46.07 |
| ATOM | 992 | CZ | ARG | 129 | −11.349 | 20.900 | 63.338 | 1.00 | 47.32 |
| ATOM | 993 | NH1 | ARG | 129 | −10.623 | 21.162 | 64.426 | 1.00 | 47.90 |
| ATOM | 994 | NH2 | ARG | 129 | −11.245 | 21.682 | 62.284 | 1.00 | 44.26 |
| ATOM | 995 | C | ARG | 129 | −10.964 | 15.279 | 67.291 | 1.00 | 36.52 |
| ATOM | 996 | O | ARG | 129 | −11.378 | 15.471 | 68.421 | 1.00 | 37.01 |
| ATOM | 997 | N | ASN | 130 | −11.146 | 14.122 | 66.664 | 1.00 | 35.22 |
| ATOM | 998 | CA | ASN | 130 | −11.762 | 13.006 | 67.376 | 1.00 | 34.87 |
| ATOM | 999 | CB | ASN | 130 | −12.340 | 11.970 | 66.367 | 1.00 | 33.94 |
| ATOM | 1000 | CC | ASN | 130 | −13.708 | 12.406 | 65.788 | 1.00 | 34.78 |
| ATOM | 1001 | OD1 | ASN | 130 | −14.138 | 11.953 | 64.723 | 1.00 | 32.62 |
| ATOM | 1002 | ND2 | ASN | 130 | −14.386 | 13.282 | 66.507 | 1.00 | 29.90 |
| ATOM | 1003 | C | ASN | 130 | −10.679 | 12.394 | 68.285 | 1.00 | 36.22 |
| ATOM | 1004 | O | ASN | 130 | −10.954 | 11.958 | 69.403 | 1.00 | 35.32 |
| ATOM | 1005 | N | VAL | 131 | −9.439 | 12.378 | 67.803 | 1.00 | 37.20 |
| ATOM | 1006 | CA | VAL | 131 | −8.359 | 11.849 | 68.607 | 1.00 | 38.51 |
| ATOM | 1007 | CB | VAL | 131 | −7.044 | 11.745 | 67.811 | 1.00 | 38.07 |
| ATOM | 1008 | CG1 | VAL | 131 | −5.916 | 11.279 | 68.733 | 1.00 | 38.68 |
| ATOM | 1009 | CG2 | VAL | 131 | −7.215 | 10.797 | 66.651 | 1.00 | 37.75 |
| ATOM | 1010 | C | VAL | 131 | −8.148 | 12.816 | 69.782 | 1.00 | 40.98 |
| ATOM | 1011 | O | VAL | 131 | −8.009 | 12.364 | 70.930 | 1.00 | 40.00 |
| ATOM | 1012 | N | ARG | 132 | −8.141 | 14.130 | 69.499 | 1.00 | 40.45 |
| ATOM | 1013 | CA | ARG | 132 | −7.943 | 15.133 | 70.547 | 1.00 | 42.82 |
| ATOM | 1014 | CB | ARG | 132 | −8.178 | 16.559 | 70.046 | 1.00 | 42.45 |
| ATOM | 1015 | CC | ARG | 132 | −8.001 | 17.603 | 71.165 | 1.00 | 48.52 |
| ATOM | 1016 | CD | ARG | 132 | −8.630 | 19.001 | 70.918 | 1.00 | 50.87 |
| ATOM | 1017 | NE | ARG | 132 | −10.080 | 18.961 | 71.107 | 1.00 | 57.79 |
| ATOM | 1018 | CZ | ARG | 132 | −10.961 | 18.537 | 70.198 | 1.00 | 59.32 |
| ATOM | 1019 | NH1 | ARG | 132 | −10.562 | 18.114 | 69.011 | 1.00 | 62.98 |
| ATOM | 1020 | NH2 | ARG | 132 | −12.246 | 18.467 | 70.501 | 1.00 | 59.20 |
| ATOM | 1021 | C | ARG | 132 | −8.935 | 14.855 | 71.658 | 1.00 | 44.60 |
| ATOM | 1022 | O | ARG | 132 | −8.572 | 14.723 | 72.828 | 1.00 | 42.61 |
| ATOM | 1023 | N | ARG | 133 | −10.196 | 14.767 | 71.258 | 1.00 | 46.77 |
| ATOM | 1024 | CA | ARG | 133 | −11.300 | 14.497 | 72.162 | 1.00 | 50.55 |
| ATOM | 1025 | CB | ARG | 133 | −12.582 | 14.317 | 71.351 | 1.00 | 52.21 |
| ATOM | 1026 | CC | ARG | 133 | −13.848 | 14.227 | 72.170 | 1.00 | 57.48 |
| ATOM | 1027 | CD | ARG | 133 | −15.000 | 13.921 | 71.244 | 1.00 | 63.69 |
| ATOM | 1028 | NE | ARG | 133 | −14.973 | 14.767 | 70.047 | 1.00 | 68.51 |
| ATOM | 1029 | CE | ARG | 133 | −15.071 | 16.097 | 70.051 | 1.00 | 70.24 |
| ATOM | 1030 | NH1 | ARG | 133 | −15.206 | 16.754 | 71.194 | 1.00 | 72.00 |
| ATOM | 1031 | NH2 | ARG | 133 | −15.030 | 16.772 | 68.908 | 1.00 | 70.13 |
| ATOM | 1032 | C | ARG | 133 | −11.035 | 13.233 | 72.985 | 1.00 | 52.21 |
| ATOM | 1033 | O | ARG | 133 | −11.082 | 13.265 | 74.218 | 1.00 | 51.81 |
| ATOM | 1034 | N | ASP | 134 | −10.763 | 12.123 | 72.302 | 1.00 | 52.59 |
| ATOM | 1035 | CA | ASP | 134 | −10.505 | 10.868 | 72.989 | 1.00 | 54.66 |
| ATOM | 1036 | CB | ASP | 134 | −10.116 | 9.772 | 72.003 | 1.00 | 56.25 |
| ATOM | 1037 | CG | ASP | 134 | −9.959 | 8.427 | 72.682 | 1.00 | 59.16 |
| ATOM | 1038 | OD1 | ASP | 134 | −10.953 | 7.972 | 73.290 | 1.00 | 60.25 |
| ATOM | 1039 | OD2 | ASP | 134 | −8.860 | 7.830 | 72.622 | 1.00 | 58.00 |
| ATOM | 1040 | C | ASP | 134 | −9.404 | 11.007 | 74.028 | 1.00 | 55.18 |
| ATOM | 1041 | O | ASP | 134 | −9.400 | 10.306 | 75.036 | 1.00 | 54.08 |
| ATOM | 1042 | N | ALA | 135 | −8.454 | 11.895 | 73.768 | 1.00 | 56.31 |
| ATOM | 1043 | CA | ALA | 135 | −7.369 | 12.133 | 74.714 | 1.00 | 57.14 |
| ATOM | 1044 | CB | ALA | 135 | −6.267 | 12.944 | 74.057 | 1.00 | 55.72 |
| ATOM | 1045 | C | ALA | 135 | −7.954 | 12.902 | 75.896 | 1.00 | 57.58 |
| ATOM | 1046 | O | ALA | 135 | −7.872 | 12.462 | 77.040 | 1.00 | 57.83 |
| ATOM | 1047 | N | ASN | 136 | −8.549 | 14.053 | 75.599 | 1.00 | 59.12 |
| ATOM | 1048 | CA | ASN | 136 | −9.176 | 14.900 | 76.610 | 1.00 | 61.05 |
| ATOM | 1049 | CB | ASN | 136 | −10.100 | 15.940 | 75.955 | 1.00 | 60.54 |
| ATOM | 1050 | CG | ASN | 136 | −9.392 | 16.808 | 74.912 | 1.00 | 62.07 |
| ATOM | 1051 | OD1 | ASN | 136 | −8.439 | 17.531 | 75.220 | 1.00 | 62.74 |
| ATOM | 1052 | ND2 | ASN | 136 | −9.870 | 16.748 | 73.673 | 1.00 | 61.09 |
| ATOM | 1053 | C | ASN | 136 | −10.007 | 14.036 | 77.565 | 1.00 | 62.37 |
| ATOM | 1054 | O | ASN | 136 | −9.965 | 14.233 | 78.773 | 1.00 | 63.28 |
| ATOM | 1055 | N | ASP | 137 | −10.758 | 13.084 | 77.013 | 1.00 | 64.25 |
| ATOM | 1056 | CA | ASP | 137 | −11.601 | 12.191 | 77.812 | 1.00 | 66.76 |
| ATOM | 1057 | CB | ASP | 137 | −12.272 | 11.131 | 76.940 | 1.00 | 67.37 |
| ATOM | 1058 | CG | ASP | 137 | −13.250 | 11.714 | 75.967 | 1.00 | 68.71 |
| ATOM | 1059 | OD1 | ASP | 137 | −14.119 | 12.507 | 76.398 | 1.00 | 68.32 |
| ATOM | 1060 | OD2 | ASP | 137 | −13.156 | 11.359 | 74.772 | 1.00 | 70.08 |
| ATOM | 1061 | C | ASP | 137 | −10.821 | 11.462 | 78.889 | 1.00 | 67.84 |
| ATOM | 1062 | O | ASP | 137 | −11.079 | 11.616 | 80.081 | 1.00 | 68.36 |
| ATOM | 1063 | N | LYS | 138 | −9.887 | 10.634 | 78.449 | 1.00 | 68.15 |
| ATOM | 1064 | CA | LYS | 138 | −9.060 | 9.875 | 79.362 | 1.00 | 70.26 |
| ATOM | 1065 | CD | LYS | 138 | −7.922 | 9.208 | 78.583 | 1.00 | 68.44 |
| ATOM | 1066 | CC | LYS | 138 | −8.419 | 8.213 | 77.552 | 1.00 | 67.37 |
| ATOM | 1067 | CD | LYS | 138 | −7.279 | 7.532 | 76.846 | 1.00 | 66.77 |
| ATOM | 1068 | CE | LYS | 138 | −7.794 | 6.509 | 75.855 | 1.00 | 65.32 |
| ATOM | 1069 | NZ | LYS | 138 | −6.661 | 5.798 | 75.206 | 1.00 | 65.96 |
| ATOM | 1070 | C | LYS | 138 | −8.509 | 10.791 | 80.454 | 1.00 | 72.20 |
| ATOM | 1071 | O | LYS | 138 | −8.637 | 10.492 | 81.642 | 1.00 | 71.61 |
| ATOM | 1072 | N | VAL | 139 | −7.919 | 11.910 | 80.037 | 1.00 | 73.71 |
| ATOM | 1073 | CA | VAL | 139 | −7.341 | 12.885 | 80.955 | 1.00 | 75.93 |
| ATOM | 1074 | CB | VAL | 139 | −6.746 | 14.094 | 80.181 | 1.00 | 75.79 |
| ATOM | 1075 | CG1 | VAL | 139 | −6.124 | 15.085 | 81.160 | 1.00 | 75.22 |
| ATOM | 1076 | CG2 | VAL | 139 | −5.708 | 13.615 | 79.168 | 1.00 | 74.05 |
| ATOM | 1077 | C | VAL | 139 | −8.357 | 13.416 | 81.974 | 1.00 | 77.78 |
| ATOM | 1078 | O | VAL | 139 | −8.168 | 13.284 | 83.184 | 1.00 | 77.15 |
| ATOM | 1079 | N | LYS | 140 | −9.432 | 14.017 | 81.471 | 1.00 | 80.32 |
| ATOM | 1080 | CA | LYS | 140 | −10.486 | 14.591 | 82.309 | 1.00 | 82.58 |
| ATOM | 1081 | CD | LYS | 140 | −11.531 | 15.266 | 81.413 | 1.00 | 83.61 |
| ATOM | 1082 | CG | LYS | 140 | −12.436 | 16.253 | 82.117 | 1.00 | 84.66 |
| ATOM | 1083 | CD | LYS | 140 | −11.616 | 17.410 | 82.677 | 1.00 | 86.11 |
| ATOM | 1084 | CE | LYS | 140 | −12.504 | 18.499 | 83.265 | 1.00 | 87.30 |
| ATOM | 1085 | NZ | LYS | 140 | −13.401 | 18.007 | 84.343 | 1.00 | 87.20 |
| ATOM | 1086 | C | LYS | 140 | −11.161 | 13.524 | 83.185 | 1.00 | 83.58 |
| ATOM | 1087 | O | LYS | 140 | −11.543 | 13.788 | 84.329 | 1.00 | 82.45 |
| ATOM | 1088 | N | ALA | 141 | −11.297 | 12.321 | 82.633 | 1.00 | 85.31 |
| ATOM | 1089 | CA | ALA | 141 | −11.917 | 11.201 | 83.331 | 1.00 | 87.36 |
| ATOM | 1090 | CE | ALA | 141 | −12.222 | 10.079 | 82.349 | 1.00 | 87.57 |
| ATOM | 1091 | C | ALA | 141 | −11.022 | 10.676 | 84.442 | 1.00 | 88.79 |
| ATOM | 1092 | O | ALA | 141 | −11.479 | 10.439 | 85.555 | 1.00 | 89.29 |
| ATOM | 1093 | N | LEU | 142 | −9.745 | 10.492 | 84.130 | 1.00 | 90.16 |
| ATOM | 1094 | CA | LEU | 142 | −8.781 | 9.982 | 85.098 | 1.00 | 91.17 |
| ATOM | 1095 | CB | LEU | 142 | −7.390 | 9.942 | 84.467 | 1.00 | 90.19 |
| ATOM | 1096 | CC | LEU | 142 | −6.450 | 8.865 | 84.999 | 1.00 | 90.65 |
| ATOM | 1097 | CD1 | LEU | 142 | −7.081 | 7.503 | 84.763 | 1.00 | 90.25 |
| ATOM | 1098 | CD2 | LEU | 142 | −5.108 | 8.947 | 84.292 | 1.00 | 90.32 |
| ATOM | 1099 | C | LEU | 142 | −8.782 | 10.889 | 86.330 | 1.00 | 92.69 |
| ATOM | 1100 | O | LEU | 142 | −8.453 | 10.463 | 87.439 | 1.00 | 92.75 |
| ATOM | 1101 | N | LEU | 143 | −9.175 | 12.141 | 86.120 | 1.00 | 94.14 |
| ATOM | 1102 | CA | LEU | 143 | −9.243 | 13.134 | 87.188 | 1.00 | 95.26 |
| ATOM | 1103 | CB | LEU | 143 | −9.537 | 14.513 | 86.585 | 1.00 | 94.57 |
| ATOM | 1104 | CC | LEU | 143 | −9.684 | 15.706 | 87.531 | 1.00 | 93.49 |
| ATOM | 1105 | CD1 | LEU | 143 | −8.373 | 15.956 | 88.248 | 1.00 | 93.46 |
| ATOM | 1106 | CD2 | LEU | 143 | −10.091 | 16.933 | 86.735 | 1.00 | 92.61 |
| ATOM | 1107 | C | LEU | 143 | −10.324 | 12.789 | 88.212 | 1.00 | 96.21 |
| ATOM | 1108 | O | LEU | 143 | −10.246 | 13.195 | 89.370 | 1.00 | 95.47 |
| ATOM | 1109 | N | LYS | 144 | −11.331 | 12.036 | 87.778 | 1.00 | 98.51 |
| ATOM | 1110 | CA | LYS | 144 | −12.441 | 11.662 | 88.649 | 1.00 | 100.82 |
| ATOM | 1111 | CB | LYS | 144 | −13.506 | 10.862 | 87.880 | 1.00 | 100.58 |
| ATOM | 1112 | CG | LYS | 144 | −13.063 | 9.460 | 87.477 | 1.00 | 101.19 |
| ATOM | 1113 | CD | LYS | 144 | −14.161 | 8.663 | 86.800 | 1.00 | 101.64 |
| ATOM | 1114 | CE | LYS | 144 | −13.669 | 7.259 | 86.448 | 1.00 | 102.30 |
| ATOM | 1115 | NZ | LYS | 144 | −14.711 | 6.420 | 85.783 | 1.00 | 102.31 |
| ATOM | 1116 | C | LYS | 144 | −12.002 | 10.847 | 89.854 | 1.00 | 102.32 |
| ATOM | 1117 | O | LYS | 144 | −12.148 | 11.296 | 90.990 | 1.00 | 102.94 |
| ATOM | 1118 | N | ASP | 145 | −11.459 | 9.657 | 89.601 | 1.00 | 103.95 |
| ATOM | 1119 | CA | ASP | 145 | −11.042 | 8.758 | 90.672 | 1.00 | 105.61 |
| ATOM | 1120 | CB | ASP | 145 | −11.670 | 7.364 | 90.449 | 1.00 | 105.33 |
| ATOM | 1121 | CG | ASP | 145 | −13.200 | 7.379 | 90.493 | 1.00 | 105.16 |
| ATOM | 1122 | OD1 | ASP | 145 | −13.778 | 7.740 | 91.545 | 1.00 | 104.28 |
| ATOM | 1123 | OD2 | ASP | 145 | −13.827 | 7.028 | 89.472 | 1.00 | 104.93 |
| ATOM | 1124 | C | ASP | 145 | −9.533 | 8.600 | 90.897 | 1.00 | 106.36 |
| ATOM | 1125 | O | ASP | 145 | −9.071 | 8.609 | 92.040 | 1.00 | 106.84 |
| ATOM | 1126 | N | LYS | 146 | −8.764 | 8.475 | 89.817 | 1.00 | 107.05 |
| ATOM | 1127 | CA | LYS | 146 | −7.323 | 8.273 | 89.963 | 1.00 | 107.59 |
| ATOM | 1128 | CB | LYS | 146 | −6.829 | 7.347 | 88.840 | 1.00 | 107.69 |
| ATOM | 1129 | CC | LYS | 146 | −7.534 | 5.979 | 88.885 | 1.00 | 108.01 |
| ATOM | 1130 | CD | LYS | 146 | −6.967 | 4.952 | 87.912 | 1.00 | 107.88 |
| ATOM | 1131 | CE | LYS | 146 | −7.653 | 3.595 | 88.104 | 1.00 | 107.75 |
| ATOM | 1132 | NZ | LYS | 146 | −7.039 | 2.522 | 87.273 | 1.00 | 107.65 |
| ATOM | 1133 | C | LYS | 146 | −6.449 | 9.527 | 90.091 | 1.00 | 108.00 |
| ATOM | 1134 | O | LYS | 146 | −6.909 | 10.654 | 89.905 | 1.00 | 108.57 |
| ATOM | 1135 | N | GLU | 147 | −5.183 | 9.286 | 90.415 | 1.00 | 107.89 |
| ATOM | 1136 | CA | GLU | 147 | −4.118 | 10.269 | 90.678 | 1.00 | 107.41 |
| ATOM | 1137 | CB | GLU | 147 | −2.780 | 9.531 | 90.640 | 1.00 | 108.68 |
| ATOM | 1138 | CG | GLU | 147 | −2.465 | 8.846 | 89.314 | 1.00 | 109.90 |
| ATOM | 1139 | CD | GLU | 147 | −1.146 | 8.093 | 89.368 | 1.00 | 111.13 |
| ATOM | 1140 | OE1 | GLU | 147 | −1.020 | 7.174 | 90.206 | 1.00 | 111.54 |
| ATOM | 1141 | OE2 | GLU | 147 | −0.235 | 8.421 | 88.579 | 1.00 | 111.87 |
| ATOM | 1142 | C | GLU | 147 | −3.868 | 11.665 | 90.067 | 1.00 | 106.09 |
| ATOM | 1143 | O | GLU | 147 | −3.101 | 12.420 | 90.647 | 1.00 | 107.03 |
| ATOM | 1144 | N | ILE | 148 | −4.448 | 12.040 | 88.936 | 1.00 | 103.57 |
| ATOM | 1145 | CA | ILE | 148 | −4.121 | 13.373 | 63.414 | 1.00 | 101.47 |
| ATOM | 1146 | CB | ILE | 148 | −4.278 | 13.430 | 86.892 | 1.00 | 101.45 |
| ATOM | 1147 | CG2 | ILE | 148 | −3.702 | 14.724 | 86.364 | 1.00 | 101.52 |
| ATOM | 1148 | CG1 | ILE | 148 | −3.553 | 12.249 | 86.245 | 1.00 | 101.43 |
| ATOM | 1149 | CD1 | ILE | 148 | −4.200 | 10.917 | 86.516 | 1.00 | 101.07 |
| ATOM | 1150 | C | ILE | 148 | −4.974 | 14.484 | 89.025 | 1.00 | 100.34 |
| ATOM | 1151 | O | ILE | 148 | −6.083 | 14.222 | 89.495 | 1.00 | 100.42 |
| ATOM | 1152 | N | SER | 149 | −4.463 | 15.717 | 89.021 | 1.00 | 98.53 |
| ATOM | 1153 | CA | SER | 149 | −5.202 | 16.873 | 89.556 | 1.00 | 96.84 |
| ATOM | 1154 | CB | SER | 149 | −4.599 | 17.320 | 90.882 | 1.00 | 97.16 |
| ATOM | 1155 | OG | SER | 149 | −3.248 | 17.689 | 90.706 | 1.00 | 97.28 |
| ATOM | 1156 | C | SER | 149 | −5.203 | 18.049 | 88.579 | 1.00 | 95.62 |
| ATOM | 1157 | O | SER | 149 | −4.454 | 18.056 | 87.608 | 1.00 | 95.10 |
| ATOM | 1158 | N | GLU | 150 | −6.040 | 19.048 | 88.860 | 1.00 | 94.27 |
| ATOM | 1159 | CA | GLU | 150 | −6.215 | 20.244 | 88.028 | 1.00 | 92.89 |
| ATOM | 1160 | CB | GLU | 150 | −6.809 | 21.357 | 88.891 | 1.00 | 92.94 |
| ATOM | 1161 | CC | GLU | 150 | −8.218 | 21.047 | 89.354 | 1.00 | 93.02 |
| ATOM | 1162 | CD | GLU | 150 | −8.668 | 21.948 | 90.477 | 1.00 | 93.68 |
| ATOM | 1163 | OE1 | GLU | 150 | −8.638 | 23.186 | 90.303 | 1.00 | 94.33 |
| ATOM | 1164 | OE2 | GLU | 150 | −9.053 | 21.411 | 91.537 | 1.00 | 93.85 |
| ATOM | 1165 | C | GLU | 150 | −5.050 | 20.810 | 87.203 | 1.00 | 91.59 |
| ATOM | 1166 | O | GLU | 150 | −5.191 | 20.999 | 85.998 | 1.00 | 91.47 |
| ATOM | 1167 | N | ASP | 151 | −3.919 | 21.092 | 87.839 | 1.00 | 90.68 |
| ATOM | 1168 | CA | ASP | 151 | −2.770 | 21.644 | 87.116 | 1.00 | 89.65 |
| ATOM | 1169 | CB | ASP | 151 | −1.743 | 22.205 | 88.091 | 1.00 | 89.29 |
| ATOM | 1170 | CG | ASP | 151 | −2.240 | 23.431 | 88.812 | 1.00 | 89.52 |
| ATOM | 1171 | OD1 | ASP | 151 | −2.566 | 24.421 | 83.129 | 1.00 | 88.83 |
| ATOM | 1172 | OD2 | ASP | 151 | −2.304 | 23.408 | 90.059 | 1.00 | 90.30 |
| ATOM | 1173 | C | ASP | 151 | −2.080 | 20.661 | 86.178 | 1.00 | 88.62 |
| ATOM | 1174 | O | ASP | 151 | −1.342 | 21.065 | 85.284 | 1.00 | 89.06 |
| ATOM | 1175 | N | ASP | 152 | −2.304 | 19.373 | 86.394 | 1.00 | 87.65 |
| ATOM | 1176 | CA | ASP | 152 | −1.707 | 18.363 | 85.535 | 1.00 | 87.45 |
| ATOM | 1177 | CB | ASP | 152 | −1.480 | 17.072 | 86.324 | 1.00 | 89.17 |
| ATOM | 1178 | CG | ASP | 152 | −0.664 | 16.052 | 85.552 | 1.00 | 91.93 |
| ATOM | 1179 | OD1 | ASP | 152 | −1.036 | 15.721 | 84.403 | 1.00 | 93.10 |
| ATOM | 1180 | OD2 | ASP | 152 | −0.351 | 15.577 | 86.102 | 1.00 | 93.68 |
| ATOM | 1181 | C | ASP | 152 | −2.700 | 18.127 | 84.388 | 1.00 | 85.95 |
| ATOM | 1182 | O | ASP | 152 | −2.324 | 17.698 | 83.295 | 1.00 | 85.41 |
| ATOM | 1183 | N | ASP | 153 | −3.969 | 18.422 | 84.670 | 1.00 | 83.73 |
| ATOM | 1184 | CA | ASP | 153 | −5.066 | 18.284 | 83.720 | 1.00 | 80.91 |
| ATOM | 1185 | CB | ASP | 153 | −6.409 | 18.406 | 84.458 | 1.00 | 80.84 |
| ATOM | 1186 | CC | ASP | 153 | −7.619 | 18.190 | 83.552 | 1.00 | 79.88 |
| ATOM | 1187 | OD1 | ASP | 153 | −8.752 | 18.336 | 84.056 | 1.00 | 79.70 |
| ATOM | 1188 | OD2 | ASP | 153 | −7.452 | 17.873 | 82.355 | 1.00 | 79.02 |
| ATOM | 1189 | C | ASP | 153 | −4.907 | 19.431 | 82.740 | 1.00 | 80.07 |
| ATOM | 1190 | O | ASP | 153 | −4.879 | 19.227 | 81.526 | 1.00 | 79.99 |
| ATOM | 1191 | N | ARG | 154 | −4.790 | 20.639 | 83.284 | 1.00 | 77.88 |
| ATOM | 1192 | CA | ARG | 154 | −4.618 | 21.833 | 82.471 | 1.00 | 76.70 |
| ATOM | 1193 | CB | ARG | 154 | −4.318 | 23.040 | 83.370 | 1.00 | 78.40 |
| ATOM | 1194 | CC | ARG | 154 | −5.457 | 23.397 | 84.310 | 1.00 | 81.12 |
| ATOM | 1195 | CO | ARG | 154 | −5.182 | 24.639 | 85.155 | 1.00 | 82.69 |
| ATOM | 1196 | NE | ARG | 154 | −6.336 | 24.979 | 85.989 | 1.00 | 84.80 |
| ATOM | 1197 | CZ | ARG | 154 | −6.376 | 25.999 | 86.843 | 1.00 | 86.51 |
| ATOM | 1198 | NH1 | ARG | 154 | −5.320 | 26.794 | 86.983 | 1.00 | 87.33 |
| ATOM | 1199 | NH2 | ARG | 154 | −7.470 | 26.225 | 87.561 | 1.00 | 86.40 |
| ATOM | 1200 | C | ARG | 154 | −3.498 | 21.658 | 81.442 | 1.00 | 74.53 |
| ATOM | 1201 | O | ARG | 154 | −3.743 | 21.703 | 80.236 | 1.00 | 73.89 |
| ATOM | 1202 | N | ARG | 155 | −2.274 | 21.463 | 81.935 | 1.00 | 72.04 |
| ATOM | 1203 | CA | ARG | 155 | −1.091 | 21.279 | 81.096 | 1.00 | 68.48 |
| ATOM | 1204 | CB | ARG | 155 | 0.122 | 20.941 | 81.964 | 1.00 | 69.87 |
| ATOM | 1205 | CC | ARG | 155 | 0.724 | 22.109 | 82.723 | 1.00 | 71.53 |
| ATOM | 1206 | CD | ARG | 155 | 1.847 | 21.617 | 83.630 | 1.00 | 73.51 |
| ATOM | 1207 | NE | ARG | 155 | 2.733 | 22.696 | 84.054 | 1.00 | 74.79 |
| ATOM | 1208 | CZ | ARG | 155 | 3.518 | 23.385 | 83.228 | 1.00 | 75.80 |
| ATOM | 1209 | NH1 | ARG | 155 | 3.531 | 23.113 | 81.927 | 1.00 | 75.28 |
| ATOM | 1210 | NH2 | ARG | 155 | 4.299 | 24.347 | 83.702 | 1.00 | 76.38 |
| ATOM | 1211 | C | ARG | 155 | −1.288 | 20.178 | 80.074 | 1.00 | 65.87 |
| ATOM | 1212 | O | ARG | 155 | −1.017 | 20.366 | 78.895 | 1.00 | 65.37 |
| ATOM | 1213 | N | SER | 156 | −1.745 | 19.022 | 80.542 | 1.00 | 63.16 |
| ATOM | 1214 | CA | SER | 156 | −1.993 | 17.882 | 79.676 | 1.00 | 60.61 |
| ATOM | 1215 | CB | SER | 156 | −2.509 | 16.702 | 80.490 | 1.00 | 58.93 |
| ATOM | 1216 | CG | SER | 156 | −2.937 | 15.672 | 79.622 | 1.00 | 59.68 |
| ATOM | 1217 | C | SER | 156 | −3.008 | 18.224 | 78.591 | 1.00 | 59.40 |
| ATOM | 1218 | O | SER | 156 | −2.854 | 17.810 | 77.446 | 1.00 | 58.17 |
| ATOM | 1219 | N | GLN | 157 | −4.046 | 18.969 | 78.965 | 1.00 | 58.38 |
| ATOM | 1220 | CA | GLN | 157 | −5.077 | 19.364 | 78.021 | 1.00 | 58.88 |
| ATOM | 1221 | CB | GLN | 157 | −6.259 | 20.012 | 78.745 | 1.00 | 61.05 |
| ATOM | 1222 | CC | GLN | 157 | −7.047 | 19.058 | 79.649 | 1.00 | 64.28 |
| ATOM | 1223 | CD | GLN | 157 | −7.648 | 17.870 | 78.894 | 1.00 | 66.97 |
| ATOM | 1224 | OE1 | GLN | 157 | −6.932 | 17.108 | 78.228 | 1.00 | 67.14 |
| ATOM | 1225 | NE2 | GLN | 157 | −8.965 | 17.702 | 79.006 | 1.00 | 67.00 |
| ATOM | 1226 | C | GLN | 157 | −4.492 | 20.327 | 77.009 | 1.00 | 59.44 |
| ATOM | 1227 | O | GLN | 157 | −4.795 | 20.247 | 75.818 | 1.00 | 60.58 |
| ATOM | 1228 | N | ASP | 158 | −3.638 | 21.226 | 77.482 | 1.00 | 57.75 |
| ATOM | 1229 | CA | ASP | 158 | −2.992 | 22.194 | 76.611 | 1.00 | 57.99 |
| ATOM | 1230 | CB | ASP | 158 | −2.371 | 23.334 | 77.447 | 1.00 | 59.91 |
| ATOM | 1231 | CC | ASP | 158 | −3.427 | 24.269 | 78.068 | 1.00 | 63.30 |
| ATOM | 1232 | OD1 | ASP | 158 | −3.051 | 25.214 | 78.805 | 1.00 | 65.17 |
| ATOM | 1233 | OD2 | ASP | 158 | −4.635 | 24.070 | 77.815 | 1.00 | 64.66 |
| ATOM | 1234 | C | ASP | 158 | −1.925 | 21.543 | 75.703 | 1.00 | 56.75 |
| ATOM | 1235 | O | ASP | 158 | −1.823 | 21.902 | 74.525 | 1.00 | 53.72 |
| ATOM | 1236 | N | ASP | 159 | −1.140 | 20.600 | 76.241 | 1.00 | 55.82 |
| ATOM | 1237 | CA | ASP | 159 | −0.113 | 19.925 | 75.441 | 1.00 | 54.58 |
| ATOM | 1238 | CB | ASP | 159 | 0.695 | 18.888 | 76.250 | 1.00 | 58.46 |
| ATOM | 1239 | CC | ASP | 159 | 1.498 | 19.499 | 77.402 | 1.00 | 62.06 |
| ATOM | 1240 | OD1 | ASP | 159 | 2.156 | 20.552 | 77.223 | 1.00 | 63.94 |
| ATOM | 1241 | OD2 | ASP | 159 | 1.500 | 18.891 | 78.497 | 1.00 | 65.22 |
| ATOM | 1242 | C | ASP | 159 | −0.813 | 19.197 | 74.301 | 1.00 | 52.55 |
| ATOM | 1243 | O | ASP | 159 | −0.320 | 19.187 | 73.174 | 1.00 | 52.06 |
| ATOM | 1244 | N | VAL | 160 | −1.963 | 18.592 | 74.606 | 1.00 | 49.12 |
| ATOM | 1245 | CA | VAL | 160 | −2.759 | 17.870 | 73.603 | 1.00 | 49.39 |
| ATOM | 1246 | CB | VAL | 160 | −3.943 | 17.083 | 74.265 | 1.00 | 49.22 |
| ATOM | 1247 | CG1 | VAL | 160 | −4.896 | 16.550 | 73.202 | 1.00 | 48.51 |
| ATOM | 1248 | CG2 | VAL | 160 | −3.402 | 15.922 | 75.096 | 1.00 | 48.41 |
| ATOM | 1249 | C | VAL | 160 | −3.330 | 18.831 | 72.548 | 1.00 | 47.61 |
| ATOM | 1250 | O | VAL | 160 | −3.330 | 18.524 | 71.348 | 1.00 | 45.78 |
| ATOM | 1251 | N | GLN | 161 | −3.820 | 19.982 | 73.008 | 1.00 | 46.87 |
| ATOM | 1252 | CA | GLN | 161 | −4.385 | 21.007 | 72.121 | 1.00 | 47.61 |
| ATOM | 1253 | CB | GLN | 161 | −4.829 | 22.234 | 72.930 | 1.00 | 49.79 |
| ATOM | 1254 | CC | GLN | 161 | −5.289 | 23.402 | 72.059 | 1.00 | 48.02 |
| ATOM | 1255 | CD | GLN | 161 | −6.388 | 22.993 | 71.113 | 1.00 | 49.19 |
| ATOM | 1256 | OE1 | GLN | 161 | −7.441 | 22.526 | 71.540 | 1.00 | 49.82 |
| ATOM | 1257 | NE2 | GLN | 161 | −6.150 | 23.156 | 69.813 | 1.00 | 50.57 |
| ATOM | 1258 | C | GLN | 161 | −3.356 | 21.461 | 71.088 | 1.00 | 46.12 |
| ATOM | 1259 | O | GLN | 161 | −3.680 | 21.734 | 69.931 | 1.00 | 45.79 |
| ATOM | 1260 | N | LYS | 162 | −2.113 | 21.544 | 71.535 | 1.00 | 44.41 |
| ATOM | 1261 | CA | LYS | 162 | −1.026 | 21.969 | 70.687 | 1.00 | 44.22 |
| ATOM | 1262 | CB | LYS | 162 | 0.173 | 22.336 | 71.572 | 1.00 | 46.80 |
| ATOM | 1263 | CG | LYS | 162 | −0.151 | 23.474 | 72.548 | 1.00 | 48.45 |
| ATOM | 1264 | CD | LYS | 162 | 1.042 | 23.861 | 73.410 | 1.00 | 55.00 |
| ATOM | 1265 | CE | LYS | 162 | 0.782 | 25.121 | 74.240 | 1.00 | 55.12 |
| ATOM | 1266 | NZ | LYS | 162 | −0.428 | 24.984 | 75.112 | 1.00 | 58.91 |
| ATOM | 1267 | C | LYS | 162 | −0.696 | 20.868 | 69.681 | 1.00 | 42.73 |
| ATOM | 1268 | O | LYS | 162 | −0.371 | 21.148 | 68.538 | 1.00 | 44.92 |
| ATOM | 1269 | N | LEU | 163 | −0.784 | 19.613 | 70.110 | 1.00 | 41.72 |
| ATOM | 1270 | CA | LEU | 163 | −0.545 | 18.492 | 69.213 | 1.00 | 38.98 |
| ATOM | 1271 | CB | LEU | 163 | −0.664 | 17.170 | 69.962 | 1.00 | 36.52 |
| ATOM | 1272 | CC | LEU | 163 | 0.584 | 16.760 | 70.740 | 1.00 | 40.24 |
| ATOM | 1273 | CD1 | LEU | 163 | 0.308 | 15.468 | 71.517 | 1.00 | 39.87 |
| ATOM | 1274 | CD2 | LEU | 163 | 1.757 | 16.585 | 69.753 | 1.00 | 36.04 |
| ATOM | 1275 | C | LEU | 163 | −1.584 | 18.550 | 68.102 | 1.00 | 37.77 |
| ATOM | 1276 | O | LEU | 163 | −1.290 | 18.274 | 66.950 | 1.00 | 37.29 |
| ATOM | 1277 | N | THR | 164 | −2.808 | 18.907 | 68.476 | 1.00 | 38.49 |
| ATOM | 1278 | CA | THR | 164 | −3.914 | 19.038 | 67.538 | 1.00 | 37.80 |
| ATOM | 1279 | CB | THR | 164 | −5.209 | 19.367 | 68.298 | 1.00 | 38.14 |
| ATOM | 1280 | OG1 | THR | 164 | −5.405 | 18.386 | 69.321 | 1.00 | 39.84 |
| ATOM | 1281 | OG2 | THR | 164 | −6.412 | 19.353 | 67.366 | 1.00 | 36.51 |
| ATOM | 1282 | C | THR | 164 | −3.584 | 20.170 | 66.562 | 1.00 | 38.02 |
| ATOM | 1283 | O | THR | 164 | −3.753 | 20.016 | 65.356 | 1.00 | 38.15 |
| ATOM | 1284 | N | ASP | 165 | −3.097 | 21.290 | 67.100 | 1.00 | 38.34 |
| ATOM | 1285 | CA | ASP | 165 | −2.709 | 22.463 | 66.307 | 1.00 | 42.07 |
| ATOM | 1286 | CB | ASP | 165 | −2.118 | 23.573 | 67.205 | 1.00 | 45.79 |
| ATOM | 1287 | CC | ASP | 165 | −3.139 | 24.181 | 68.158 | 1.00 | 49.06 |
| ATOM | 1288 | OD1 | ASP | 165 | −2.716 | 24.949 | 69.057 | 1.00 | 51.38 |
| ATOM | 1289 | OD2 | ASP | 165 | −4.355 | 23.905 | 68.005 | 1.00 | 48.41 |
| ATOM | 1290 | C | ASP | 165 | −1.670 | 22.124 | 65.251 | 1.00 | 40.17 |
| ATOM | 1291 | O | ASP | 165 | −1.809 | 22.489 | 64.093 | 1.00 | 39.87 |
| ATOM | 1292 | N | ALA | 166 | −0.607 | 21.443 | 65.658 | 1.00 | 39.77 |
| ATOM | 1293 | CA | ALA | 166 | 0.430 | 21.079 | 64.699 | 1.00 | 38.11 |
| ATOM | 1294 | CB | ALA | 166 | 1.659 | 20.514 | 65.439 | 1.00 | 36.43 |
| ATOM | 1295 | C | ALA | 166 | −0.096 | 20.067 | 63.663 | 1.00 | 35.40 |
| ATOM | 1296 | O | ALA | 166 | 0.291 | 20.104 | 62.492 | 1.00 | 36.48 |
| ATOM | 1297 | N | ALA | 167 | −0.976 | 19.165 | 64.086 | 1.00 | 32.91 |
| ATOM | 1298 | CA | ALA | 167 | −1.506 | 18.188 | 63.148 | 1.00 | 32.56 |
| ATOM | 1299 | CB | ALA | 167 | −2.260 | 17.075 | 63.900 | 1.00 | 27.68 |
| ATOM | 1300 | C | ALA | 167 | −2.434 | 18.925 | 62.167 | 1.00 | 33.32 |
| ATOM | 1301 | O | ALA | 167 | −2.378 | 18.708 | 60.957 | 1.00 | 32.20 |
| ATOM | 1302 | N | ILE | 168 | −3.276 | 19.815 | 62.690 | 1.00 | 33.15 |
| ATOM | 1303 | CA | ILE | 168 | −4.173 | 20.549 | 61.817 | 1.00 | 34.96 |
| ATOM | 1304 | CB | ILE | 168 | −5.224 | 21.422 | 62.650 | 1.00 | 36.24 |
| ATOM | 1305 | CG2 | ILE | 168 | −5.269 | 22.847 | 62.144 | 1.00 | 33.31 |
| ATOM | 1306 | CG1 | ILE | 168 | −6.660 | 20.870 | 62.468 | 1.00 | 38.04 |
| ATOM | 1307 | CD1 | ILE | 168 | −6.924 | 19.486 | 62.974 | 1.00 | 35.50 |
| ATOM | 1308 | C | ILE | 168 | −3.318 | 21.409 | 60.882 | 1.00 | 34.13 |
| ATOM | 1309 | O | ILE | 168 | −3.619 | 21.546 | 59.707 | 1.00 | 32.89 |
| ATOM | 1310 | N | LYS | 169 | −2.221 | 21.957 | 61.389 | 1.00 | 37.28 |
| ATOM | 1311 | CA | LYS | 169 | −1.364 | 22.798 | 60.539 | 1.00 | 39.15 |
| ATOM | 1312 | CB | LYS | 169 | −0.261 | 23.461 | 61.365 | 1.00 | 43.88 |
| ATOM | 1313 | CC | LYS | 169 | 0.749 | 24.246 | 60.509 | 1.00 | 48.23 |
| ATOM | 1314 | CD | LYS | 169 | 1.813 | 24.917 | 61.371 | 1.00 | 52.07 |
| ATOM | 1315 | CE | LYS | 169 | 2.902 | 25.630 | 60.530 | 1.00 | 54.04 |
| ATOM | 1316 | NZ | LYS | 169 | 2.387 | 26.724 | 59.637 | 1.00 | 55.30 |
| ATOM | 1317 | C | LYS | 169 | −0.744 | 22.059 | 59.342 | 1.00 | 36.63 |
| ATOM | 1318 | O | LYS | 169 | −0.649 | 22.616 | 58.258 | 1.00 | 36.33 |
| ATOM | 1319 | N | LYS | 170 | −0.336 | 20.808 | 59.525 | 1.00 | 36.30 |
| ATOM | 1320 | CA | LYS | 170 | 0.248 | 20.042 | 58.412 | 1.00 | 35.20 |
| ATOM | 1321 | CB | LYS | 170 | 0.798 | 18.694 | 58.907 | 1.00 | 35.03 |
| ATOM | 1322 | CG | LYS | 170 | 2.026 | 18.786 | 59.780 | 1.00 | 35.34 |
| ATOM | 1323 | CD | LYS | 170 | 2.268 | 17.475 | 60.522 | 1.00 | 37.56 |
| ATOM | 1324 | CE | LYS | 170 | 3.459 | 17.597 | 61.468 | 1.00 | 43.01 |
| ATOM | 1325 | NZ | LYS | 170 | 3.540 | 16.490 | 62.471 | 1.00 | 43.46 |
| ATOM | 1326 | C | LYS | 170 | −0.797 | 19.788 | 57.322 | 1.00 | 34.52 |
| ATOM | 1327 | O | LYS | 170 | −0.503 | 19.889 | 56.140 | 1.00 | 36.16 |
| ATOM | 1328 | N | ILE | 171 | −2.013 | 19.439 | 57.734 | 1.00 | 34.26 |
| ATOM | 1329 | CA | ILE | 171 | −3.102 | 19.180 | 56.802 | 1.00 | 33.78 |
| ATOM | 1330 | CB | ILE | 171 | −4.433 | 18.788 | 57.543 | 1.00 | 31.23 |
| ATOM | 1331 | CG2 | ILE | 171 | −5.614 | 18.890 | 56.563 | 1.00 | 28.07 |
| ATOM | 1332 | CG1 | ILE | 171 | −4.312 | 17.386 | 58.162 | 1.00 | 26.76 |
| ATOM | 1333 | CD1 | ILE | 171 | −5.344 | 17.070 | 59.301 | 1.00 | 20.37 |
| ATOM | 1334 | C | ILE | 171 | −3.340 | 20.430 | 55.977 | 1.00 | 32.17 |
| ATOM | 1335 | O | ILE | 171 | −3.469 | 20.358 | 54.769 | 1.00 | 34.63 |
| ATOM | 1336 | N | GLU | 172 | −3.379 | 21.580 | 56.629 | 1.00 | 34.69 |
| ATOM | 1337 | CA | GLU | 172 | −3.606 | 22.839 | 55.918 | 1.00 | 35.82 |
| ATOM | 1338 | CB | GLU | 172 | −3.678 | 24.007 | 56.911 | 1.00 | 37.88 |
| ATOM | 1339 | CG | GLU | 172 | −4.713 | 23.810 | 58.011 | 1.00 | 43.69 |
| ATOM | 1340 | CD | GLU | 172 | −6.141 | 23.889 | 57.503 | 1.00 | 47.30 |
| ATOM | 1341 | OE1 | GLU | 172 | −6.339 | 23.631 | 56.293 | 1.00 | 49.76 |
| ATOM | 1342 | OE2 | GLU | 172 | −7.058 | 24.193 | 58.312 | 1.00 | 47.70 |
| ATOM | 1343 | C | GLU | 172 | −2.494 | 23.092 | 54.895 | 1.00 | 36.71 |
| ATOM | 1344 | O | GLU | 172 | −2.769 | 23.503 | 53.756 | 1.00 | 38.19 |
| ATOM | 1345 | N | ALA | 173 | −1.241 | 22.839 | 55.284 | 1.00 | 37.14 |
| ATOM | 1346 | CA | ALA | 173 | −0.103 | 23.053 | 54.367 | 1.00 | 37.45 |
| ATOM | 1347 | CB | ALA | 173 | 1.230 | 22.912 | 55.109 | 1.00 | 36.11 |
| ATOM | 1348 | C | ALA | 173 | −0.153 | 22.079 | 53.201 | 1.00 | 36.09 |
| ATOM | 1349 | O | ALA | 173 | 0.136 | 22.428 | 52.066 | 1.00 | 39.05 |
| ATOM | 1350 | N | ALA | 174 | −0.513 | 20.843 | 53.472 | 1.00 | 35.66 |
| ATOM | 1351 | CA | ALA | 174 | −0.598 | 19.905 | 52.375 | 1.00 | 36.41 |
| ATOM | 1352 | CB | ALA | 174 | −0.873 | 18.518 | 52.901 | 1.00 | 36.97 |
| ATOM | 1353 | C | ALA | 174 | −1.715 | 20.363 | 51.419 | 1.00 | 33.92 |
| ATOM | 1354 | O | ALA | 174 | −1.558 | 20.279 | 50.213 | 1.00 | 33.46 |
| ATOM | 1355 | N | LEU | 175 | −2.827 | 20.862 | 51.970 | 1.00 | 34.46 |
| ATOM | 1356 | CA | LEU | 175 | −3.958 | 21.331 | 51.162 | 1.00 | 33.61 |
| ATOM | 1357 | CB | LEU | 175 | −5.154 | 21.716 | 52.058 | 1.00 | 34.33 |
| ATOM | 1358 | CG | LEU | 175 | −6.356 | 22.318 | 51.296 | 1.00 | 32.56 |
| ATOM | 1359 | CD1 | LEU | 175 | −6.771 | 21.397 | 50.123 | 1.00 | 28.31 |
| ATOM | 1360 | CD2 | LEU | 175 | −7.512 | 22.540 | 52.259 | 1.00 | 29.36 |
| ATOM | 1361 | C | LEU | 175 | −3.612 | 22.518 | 50.263 | 1.00 | 33.20 |
| ATOM | 1362 | O | LEU | 175 | −3.888 | 22.503 | 49.064 | 1.00 | 31.43 |
| ATOM | 1363 | N | ALA | 176 | −3.012 | 23.542 | 50.863 | 1.00 | 34.82 |
| ATOM | 1364 | CA | ALA | 176 | −2.616 | 24.746 | 50.146 | 1.00 | 34.42 |
| ATOM | 1365 | CB | ALA | 176 | −1.953 | 25.712 | 51.106 | 1.00 | 35.13 |
| ATOM | 1366 | C | ALA | 176 | −1.668 | 24.418 | 48.989 | 1.00 | 37.83 |
| ATOM | 1367 | O | ALA | 176 | −1.868 | 24.882 | 47.866 | 1.00 | 38.78 |
| ATOM | 1368 | N | ASP | 177 | −0.630 | 23.627 | 49.248 | 1.00 | 39.20 |
| ATOM | 1369 | CA | ASP | 177 | 0.281 | 23.279 | 48.171 | 1.00 | 41.20 |
| ATOM | 1370 | CB | ASP | 177 | 1.397 | 22.365 | 48.664 | 1.00 | 45.40 |
| ATOM | 1371 | CG | ASP | 177 | 2.318 | 23.065 | 49.640 | 1.00 | 53.11 |
| ATOM | 1372 | OD1 | ASP | 177 | 2.689 | 24.232 | 49.361 | 1.00 | 54.79 |
| ATOM | 1373 | OD2 | ASP | 177 | 2.687 | 22.457 | 50.674 | 1.00 | 55.87 |
| ATOM | 1374 | C | ASP | 177 | −0.473 | 22.597 | 47.047 | 1.00 | 41.24 |
| ATOM | 1375 | O | ASP | 177 | −0.196 | 22.834 | 45.872 | 1.00 | 39.86 |
| ATOM | 1376 | N | LYS | 178 | −1.433 | 21.749 | 47.408 | 1.00 | 41.20 |
| ATOM | 1377 | CA | LYS | 178 | −2.226 | 21.049 | 46.402 | 1.00 | 42.30 |
| ATOM | 1378 | CB | LYS | 178 | −3.077 | 19.959 | 47.065 | 1.00 | 44.35 |
| ATOM | 1379 | CG | LYS | 178 | −3.957 | 19.180 | 46.096 | 1.00 | 44.13 |
| ATOM | 1380 | CD | LYS | 178 | −3.153 | 18.478 | 45.013 | 1.00 | 41.10 |
| ATOM | 1381 | CE | LYS | 178 | −2.255 | 17.422 | 45.586 | 1.00 | 39.67 |
| ATOM | 1382 | NZ | LYS | 178 | −1.734 | 16.546 | 44.503 | 1.00 | 41.20 |
| ATOM | 1383 | C | LYS | 178 | −3.105 | 22.011 | 45.584 | 1.00 | 41.77 |
| ATOM | 1384 | O | LYS | 178 | −3.145 | 21.916 | 44.365 | 1.00 | 43.66 |
| ATOM | 1385 | N | GLU | 179 | −3.802 | 22.942 | 46.227 | 1.00 | 41.77 |
| ATOM | 1386 | CA | GLU | 179 | −4.601 | 23.892 | 45.453 | 1.00 | 43.66 |
| ATOM | 1387 | CB | GLU | 179 | −5.490 | 24.756 | 46.351 | 1.00 | 43.35 |
| ATOM | 1388 | CG | GLU | 179 | −6.538 | 23.967 | 47.098 | 1.00 | 47.58 |
| ATOM | 1389 | CD | GLU | 179 | −7.658 | 24.835 | 47.654 | 1.00 | 49.87 |
| ATOM | 1390 | OE1 | GLU | 179 | −8.292 | 25.571 | 46.855 | 1.00 | 51.89 |
| ATOM | 1391 | OE2 | GLU | 179 | −7.916 | 24.775 | 48.880 | 1.00 | 48.60 |
| ATOM | 1392 | C | GLU | 179 | −3.685 | 24.797 | 44.629 | 1.00 | 44.17 |
| ATOM | 1393 | O | GLU | 179 | −4.041 | 25.192 | 43.526 | 1.00 | 46.42 |
| ATOM | 1394 | N | ALA | 180 | −2.511 | 25.132 | 45.163 | 1.00 | 44.61 |
| ATOM | 1395 | CA | ALA | 180 | −1.559 | 25.979 | 44.438 | 1.00 | 45.76 |
| ATOM | 1396 | CB | ALA | 180 | −0.359 | 26.289 | 45.300 | 1.00 | 43.35 |
| ATOM | 1397 | C | ALA | 180 | −1.129 | 25.217 | 43.197 | 1.00 | 47.11 |
| ATOM | 1398 | O | ALA | 180 | −0.914 | 25.792 | 42.133 | 1.00 | 48.59 |
| ATOM | 1399 | N | GLU | 181 | −1.028 | 23.903 | 43.352 | 1.00 | 47.73 |
| ATOM | 1400 | CA | GLU | 181 | −0.651 | 23.002 | 42.277 | 1.00 | 48.37 |
| ATOM | 1401 | CB | GLU | 181 | −0.452 | 21.600 | 42.874 | 1.00 | 50.12 |
| ATOM | 1402 | CG | GLU | 181 | 0.477 | 20.663 | 42.114 | 1.00 | 50.74 |
| ATOM | 1403 | CD | GLU | 181 | 0.533 | 19.276 | 42.751 | 1.00 | 51.08 |
| ATOM | 1404 | OE1 | GLU | 181 | −0.476 | 18.535 | 42.693 | 1.00 | 50.45 |
| ATOM | 1405 | OE2 | GLU | 181 | 1.584 | 18.933 | 43.324 | 1.00 | 49.78 |
| ATOM | 1406 | C | GLU | 181 | −1.792 | 23.000 | 41.238 | 1.00 | 49.22 |
| ATOM | 1407 | O | GLU | 181 | −1.559 | 23.210 | 40.058 | 1.00 | 48.41 |
| ATOM | 1408 | N | LEU | 182 | −3.021 | 22.754 | 41.696 | 1.00 | 50.42 |
| ATOM | 1409 | CA | LEU | 182 | −4.208 | 22.735 | 40.832 | 1.00 | 51.74 |
| ATOM | 1410 | CB | LEU | 182 | −5.466 | 22.494 | 41.651 | 1.00 | 49.82 |
| ATOM | 1411 | CG | LEU | 182 | −5.941 | 21.106 | 42.089 | 1.00 | 50.S6 |
| ATOM | 1412 | CD1 | LEU | 182 | −6.212 | 20.264 | 40.85S 1.00 | 49.40 | |
| ATOM | 1413 | CD2 | LEU | 182 | −4.901 | 20.464 | 42.954 | 1.00 | 48.77 |
| ATOM | 1414 | C | LEU | 182 | −4.411 | 24.036 | 40.072 | 1.00 | 54.20 |
| ATOM | 1415 | O | LEU | 182 | −4.818 | 24.041 | 38.910 | 1.00 | 53.28 |
| ATOM | 1416 | N | MET | 183 | −4.149 | 25.134 | 40.768 | 1.00 | 56.78 |
| ATOM | 1417 | CA | MET | 183 | −4.308 | 26.475 | 40.245 | 1.00 | 60.66 |
| ATOM | 1418 | CB | MET | 183 | −4.554 | 27.413 | 41.418 | 1.00 | 61.27 |
| ATOM | 1419 | CG | MET | 183 | −4.745 | 28.857 | 41.067 | 1.00 | 63.93 |
| ATOM | 1420 | SD | MET | 183 | −4.901 | 29.814 | 42.586 | 1.00 | 68.53 |
| ATOM | 1421 | CE | MET | 183 | −6.330 | 28.989 | 43.348 | 1.00 | 67.90 |
| ATOM | 1422 | C | MET | 183 | −3.071 | 26.916 | 39.482 | 1.00 | 64.03 |
| ATOM | 1423 | O | MET | 183 | −3.052 | 27.987 | 38.869 | 1.00 | 64.46 |
| ATOM | 1424 | N | GLN | 184 | −2.042 | 26.076 | 39.514 | 1.00 | 67.44 |
| ATOM | 1425 | CA | OLN | 184 | −0.777 | 26.383 | 38.860 | 1.00 | 71.23 |
| ATOM | 1426 | CB | GLN | 184 | 0.222 | 25.242 | 39.116 | 1.00 | 73.44 |
| ATOM | 1427 | CG | GLN | 184 | 1.697 | 25.659 | 39.291 | 1.00 | 76.77 |
| ATOM | 1428 | CD | GLN | 184 | 2.348 | 26.207 | 38.020 | 1.00 | 79.02 |
| ATOM | 1429 | OE1 | GLN | 184 | 1.893 | 27.204 | 37.442 | 1.00 | 79.20 |
| ATOM | 1430 | NE2 | GLN | 184 | 3.428 | 25.554 | 37.586 | 1.00 | 79.55 |
| ATOM | 1431 | C | GLN | 184 | −0.985 | 26.598 | 37.360 | 1.00 | 72.64 |
| ATOM | 1432 | O | GLN | 184 | −0.971 | 27.737 | 36.873 | 1.00 | 72.31 |
| ATOM | 1433 | N | ALA | 185 | −1.189 | 25.507 | 36.633 | 1.00 | 74.64 |
| ATOM | 1434 | CA | ALA | 185 | −1.387 | 25.593 | 35.195 | 1.00 | 76.72 |
| ATOM | 1435 | CB | ALA | 185 | −0.351 | 24.721 | 34.473 | 1.00 | 76.74 |
| ATOM | 1436 | C | ALA | 185 | −2.800 | 25.169 | 34.812 | 1.00 | 77.63 |
| ATOM | 1437 | O | ALA | 185 | −3.510 | 25.901 | 34.116 | 1.00 | 79.25 |
| ATOM | 1438 | O1 | HOH | 302 | 5.786 | 24.929 | 33.706 | 1.00 | 46.39 |
| ATOM | 1439 | O1 | HOH | 303 | 16.711 | 7.421 | 13.716 | 1.00 | 40.03 |
| ATOM | 1440 | O1 | HOH | 304 | −0.640 | 6.780 | 57.814 | 1.00 | 49.80 |
| ATOM | 1441 | O1 | HOH | 305 | −3.664 | 8.728 | 22.988 | 1.00 | 37.04 |
| ATOM | 1442 | O1 | HOH | 307 | −6.985 | 9.376 | 71.599 | 1.00 | 44.76 |
| ATOM | 1443 | O1 | HOH | 309 | 2.439 | 23.127 | 69.818 | 1.00 | 64.96 |
| ATOM | 1444 | O1 | HOH | 310 | −12.357 | 22.793 | 59.997 | 1.00 | 49.37 |
| ATOM | 1445 | O1 | HOH | 311 | −5.000 | 9.881 | 15.800 | 1.00 | 39.08 |
| ATOM | 1446 | O1 | HOH | 312 | −1.379 | 2.409 | 21.615 | 1.00 | 37.20 |
| ATOM | 1447 | O1 | HOH | 314 | 4.226 | 11.609 | 56.051 | 1.00 | 61.48 |
| ATOM | 1448 | O1 | HOH | 315 | 3.062 | 22.032 | 74.337 | 1.00 | 57.32 |
| ATOM | 1449 | O1 | HOH | 316 | 2.291 | 19.201 | 55.609 | 1.00 | 32.55 |
| ATOM | 1450 | O1 | HOH | 317 | −14.260 | 15.606 | 65.187 | 1.00 | 48.61 |
| ATOM | 1451 | O1 | HOH | 318 | 1.372 | 26.408 | 49.218 | 1.00 | 45.06 |
| ATOM | 1452 | O1 | HOH | 320 | −6.791 | 23.072 | 66.093 | 1.00 | 55.37 |
| ATOM | 1453 | O1 | HOH | 321 | −5.960 | 5.653 | 7.525 | 1.00 | 57.42 |
| ATOM | 1454 | O1 | HOH | 322 | 9.023 | 7.824 | 4.729 | 1.00 | 49.88 |
| ATOM | 1455 | O1 | HOH | 323 | −5.164 | 25.011 | 53.787 | 1.00 | 48.29 |
| ATOM | 1456 | O1 | HOH | 324 | −7.620 | 15.194 | 35.505 | 1.00 | 42.96 |
| ATOM | 1457 | O1 | HOH | 325 | 0.761 | 16.491 | 65.949 | 1.00 | 49.53 |
| ATOM | 1458 | O1 | HOH | 327 | −13.358 | 14.152 | 48.865 | 1.00 | 49.48 |
| ATOM | 1459 | O1 | HOH | 328 | 9.574 | 23.698 | 19.099 | 1.00 | 70.52 |
| ATOM | 1460 | O1 | HOH | 329 | −15.006 | 12.044 | 46.069 | 1.00 | 61.30 |
| ATOM | 1461 | O1 | HOH | 330 | 14.950 | 11.135 | 27.309 | 1.00 | 82.65 |
| ATOM | 1462 | O1 | HOH | 331 | −6.107 | 10.675 | 18.376 | 1.00 | 43.27 |
| ATOM | 1463 | O1 | HOH | 332 | −17.369 | 21.115 | 39.828 | 1.00 | 59.43 |
| ATOM | 1464 | O1 | HOH | 333 | 3.753 | 16.179 | 57.494 | 1.00 | 71.35 |
| ATOM | 1465 | O1 | HOH | 334 | −15.680 | 10.875 | 74.742 | 1.00 | 61.46 |
| ATOM | 1466 | O1 | HOH | 335 | −15.080 | 23.421 | 48.843 | 1.00 | 55.54 |
| ATOM | 1467 | O1 | HOH | 336 | −17.698 | 17.552 | 36.236 | 1.00 | 49.63 |
| ATOM | 1468 | O1 | HOH | 337 | −3.687 | 11.077 | 13.706 | 1.00 | 31.62 |
| ATOM | 1469 | O1 | HOH | 338 | 3.927 | 6.239 | 5.123 | 1.00 | 44.26 |
| ATOM | 1470 | O1 | HOH | 339 | 2.369 | 24.751 | 52.035 | 1.00 | 50.57 |
| ATOM | 1471 | O1 | HOH | 340 | −15.269 | 15.498 | 54.910 | 1.00 | 75.04 |
| ATOM | 1472 | O1 | HOH | 341 | 15.978 | 10.746 | 6.920 | 1.00 | 59.72 |
| ATOM | 1473 | O1 | HOH | 342 | −4.143 | 18.035 | 17.666 | 1.00 | 41.15 |
| ATOM | 1474 | O1 | HOH | 343 | −14.940 | 13.136 | 37.292 | 1.00 | 58.44 |
| ATOM | 1475 | O1 | HOH | 344 | 2.563 | 12.714 | 60.213 | 1.00 | 57.07 |
| ATOM | 1476 | O1 | HOH | 346 | 1.596 | 13.582 | 86.916 | 1.00 | 70.91 |
| ATOM | 1477 | O1 | HOH | 347 | 7.838 | 16.595 | 73.384 | 1.00 | 59.18 |
| ATOM | 1478 | O1 | HOH | 348 | −6.147 | 9.577 | 11.401 | 1.00 | 57.10 |
| ATOM | 1479 | O1 | HOH | 349 | −14.722 | 27.444 | 46.158 | 1.00 | 90.16 |
| ATOM | 1480 | O1 | HOH | 350 | −4.140 | 13.336 | 31.273 | 1.00 | 33.25 |
| ATOM | 1481 | O1 | HOH | 351 | −2.060 | 25.041 | 85.618 | 1.00 | 80.45 |
| ATOM | 1482 | O1 | HOH | 352 | −11.312 | 11.026 | 39.981 | 1.00 | 45.94 |
| ATOM | 1483 | O1 | HOH | 353 | −17.003 | 20.181 | 54.859 | 1.00 | 71.86 |
| ATOM | 1484 | O1 | HOH | 354 | −0.916 | 3.322 | 71.864 | 1.00 | 49.68 |
| ATOM | 1485 | O1 | HOH | 355 | −7.005 | 20.501 | 13.831 | 1.00 | 53.66 |
| ATOM | 1486 | O1 | HOH | 356 | −14.600 | 23.546 | 55.631 | 1.00 | 48.35 |
| ATOM | 1487 | O1 | HOH | 357 | −17.819 | 23.151 | 50.976 | 1.00 | 52.11 |
| ATOM | 1488 | O1 | HOH | 358 | 2.254 | 26.577 | 21.628 | 1.00 | 60.67 |
| ATOM | 1489 | O1 | HOH | 359 | 0.803 | 16.354 | 50.390 | 1.00 | 52.48 |
| ATOM | 1490 | O1 | HOH | 360 | 17.200 | 10.484 | 22.120 | 1.00 | 51.01 |
| ATOM | 1491 | O1 | HOH | 361 | −15.864 | 16.596 | 42.676 | 1.00 | 56.63 |
| ATOM | 1492 | O1 | HOH | 362 | 2.181 | 8.776 | 24.952 | 1.00 | 38.73 |
| ATOM | 1493 | O1 | HOH | 363 | −7.968 | 23.240 | 28.701 | 1.00 | 50.39 |
| ATOM | 1494 | O1 | HOH | 364 | −4.004 | 0.134 | 10.731 | 1.00 | 90.56 |
| ATOM | 1495 | O1 | HOH | 365 | −3.704 | 10.675 | 31.678 | 1.00 | 30.70 |
| ATOM | 1496 | O1 | HOH | 366 | −13.266 | 25.262 | 52.064 | 1.00 | 62.28 |
| ATOM | 1497 | O1 | HOH | 367 | −16.916 | 18.510 | 59.343 | 1.00 | 47.69 |
| ATOM | 1498 | O1 | HOH | 368 | −3.703 | 25.280 | 60.980 | 1.00 | 53.12 |
| ATOM | 1499 | O1 | HOH | 369 | −0.275 | 16.417 | 37.735 | 1.00 | 66.01 |
| ATOM | 1500 | O1 | HOH | 370 | −7.122 | 20.168 | 74.323 | 1.00 | 40.70 |
| ATOM | 1501 | O1 | HOH | 371 | 4.079 | 24.812 | 54.526 | 1.00 | 57.35 |
| ATOM | 1502 | O1 | HOH | 373 | −9.613 | 19.800 | 80.552 | 1.00 | 64.39 |
| ATOM | 1503 | O1 | HOH | 374 | −9.716 | 25.634 | 31.190 | 1.00 | 50.17 |
| ATOM | 1504 | O1 | HOH | 375 | −0.532 | 22.524 | 36.466 | 1.00 | 61.07 |
| ATOM | 1505 | O1 | HOH | 376 | −14.409 | 13.165 | 83.189 | 1.00 | 63.05 |
| ATOM | 1506 | O1 | HOH | 377 | −11.018 | 21.644 | 53.328 | 1.00 | 44.58 |
| ATOM | 1507 | O1 | HOH | 378 | 5.370 | 26.188 | 22.596 | 1.00 | 59.67 |
| ATOM | 1508 | O1 | HOH | 380 | −13.995 | 17.778 | 73.426 | 1.00 | 79.09 |
| ATOM | 1509 | O1 | HOH | 381 | 4.885 | 0.329 | 8.120 | 1.00 | 74.85 |
| ATOM | 1510 | O1 | HOH | 382 | 1.331 | 11.313 | 6.097 | 1.00 | 47.98 |
| ATOM | 1511 | O1 | HOH | 383 | 11.281 | 12.141 | 6.394 | 1.00 | 56.27 |
| ATOM | 1512 | O1 | HOH | 400 | −13.284 | 11.946 | 58.880 | 1.00 | 47.79 |
| ATOM | 1513 | O1 | HOH | 402 | −15.563 | 20.103 | 49.349 | 1.00 | 51.23 |
| ATOM | 1514 | O1 | HOH | 403 | 2.253 | 19.239 | 4.410 | 1.00 | 73.44 |
| ATOM | 1515 | O1 | HOH | 405 | 11.304 | 2.402 | 13.475 | 1.00 | 84.55 |
| ATOM | 1516 | O1 | HOH | 406 | −0.935 | 13.768 | 34.803 | 1.00 | 48.34 |
| ATOM | 1517 | O1 | HOH | 407 | −3.810 | 21.771 | 34.390 | 1.00 | 43.81 |
| ATOM | 1518 | O1 | HOH | 408 | −3.265 | 3.486 | 47.748 | 1.00 | 66.97 |
| ATOM | 1519 | O1 | HOH | 409 | 1.104 | 9.945 | 9.880 | 1.00 | 49.13 |
| ATOM | 1520 | O1 | HOH | 410 | 0.422 | 16.427 | 34.414 | 1.00 | 87.71 |
| ATOM | 1521 | O1 | HOH | 411 | −2.673 | 0.494 | 19.885 | 1.00 | 52.04 |
| ATOM | 1522 | O1 | HOH | 412 | −3.089 | 13.895 | 92.403 | 1.00 | 59.96 |
| ATOM | 1523 | O1 | HOH | 413 | 3.194 | 24.668 | 20.476 | 1.00 | 64.45 |
| ATOM | 1524 | O1 | HOH | 415 | −8.097 | 12.619 | 54.223 | 1.00 | 40.52 |
| ATOM | 1525 | O1 | HOH | 416 | 2.795 | 7.151 | 22.607 | 1.00 | 54.61 |
| ATOM | 1526 | O1 | HOH | 417 | 3.751 | 5.165 | 25.893 | 1.00 | 44.77 |
| ATOM | 1527 | O1 | HOH | 418 | 1.622 | 9.842 | 42.243 | 1.00 | 56.21 |
| ATOM | 1528 | O1 | HOH | 419 | 0.421 | 23.884 | 77.749 | 1.00 | 61.98 |
| ATOM | 1529 | O1 | HOH | 420 | 14.627 | 6.089 | 21.772 | 1.00 | 57.19 |
| ATOM | 1530 | O1 | HOH | 421 | 13.038 | 12.816 | 22.645 | 1.00 | 64.28 |
| ATOM | 1531 | O1 | HOH | 422 | −11.444 | 26.483 | 43.160 | 1.00 | 58.77 |
| ATOM | 1532 | O1 | HOH | 423 | 4.279 | 12.562 | 88.202 | 1.00 | 62.72 |
| ATOM | 1533 | O1 | HOH | 424 | −12.414 | 14.580 | 87.763 | 1.00 | 89.26 |
| ATOM | 1534 | O1 | HOH | 425 | −16.766 | 14.530 | 45.869 | 1.00 | 96.42 |
| ATOM | 1535 | O1 | HOH | 426 | 7.867 | 11.318 | 7.942 | 1.00 | 55.18 |
| ATOM | 1536 | O1 | HOH | 427 | 4.366 | 8.492 | 6.798 | 1.00 | 70.18 |
| ATOM | 1537 | O1 | HOH | 428 | −2.106 | 3.432 | 77.575 | 1.00 | 57.89 |
| ATOM | 1538 | O1 | HOH | 429 | 18.086 | 17.462 | 19.670 | 1.00 | 70.05 |
| ATOM | 1539 | O1 | HOH | 430 | −6.737 | 8.016 | 94.166 | 1.00 | 59.01 |
| ATOM | 1540 | O1 | HOH | 431 | 6.971 | 9.475 | 58.674 | 1.00 | 80.86 |
| ATOM | 1541 | O1 | HOH | 432 | −1.536 | 19.816 | 31.738 | 1.00 | 49.54 |
| ATOM | 1542 | O1 | HOH | 433 | 7.698 | 10.437 | 31.906 | 1.00 | 61.73 |
| ATOM | 1543 | O1 | HOH | 434 | 0.207 | 20.512 | 7.972 | 1.00 | 74.94 |
| ATOM | 1544 | O1 | HOH | 435 | 3.776 | 13.746 | 6.429 | 1.00 | 75.18 |
| ATOM | 1545 | O1 | HOH | 436 | −18.571 | 19.732 | 52.567 | 1.00 | 63.53 |
| ATOM | 1546 | O1 | HOH | 437 | −1.899 | 29.425 | 52.295 | 1.00 | 55.12 |
| ATOM | 1547 | O1 | HOH | 438 | −6.880 | 6.890 | 17.770 | 1.00 | 49.51 |
| ATOM | 1548 | O1 | HOH | 439 | −6.912 | 14.453 | 37.516 | 1.00 | 51.30 |
| ATOM | 1549 | O1 | EOH | 440 | 9.467 | 16.541 | 12.773 | 1.00 | 54.26 |
| ATOM | 1550 | O1 | HOH | 441 | 2.279 | 17.814 | 53.244 | 1.00 | 61.77 |
| ATOM | 1551 | O1 | HOH | 442 | −2.650 | 0.463 | 13.822 | 1.00 | 46.01 |
| ATOM | 1552 | O1 | HOH | 443 | −3.215 | 24.966 | 74.028 | 1.00 | 62.02 |
| ATOM | 1553 | O1 | HOH | 444 | −4.683 | 14.010 | 33.817 | 1.00 | 65.38 |
| ATOM | 1554 | O1 | HOH | 445 | −8.336 | 27.942 | 89.666 | 1.00 | 52.95 |
| ATOM | 1555 | O1 | HOH | 446 | −4.502 | 12.079 | 81.403 | 1.00 | 90.88 |
| ATOM | 1556 | O1 | HOH | 447 | −9.457 | 24.981 | 58.998 | 1.00 | 52.30 |
| ATOM | 1557 | O1 | HOH | 448 | −9.756 | 25.777 | 88.998 | 1.00 | 62.22 |
| ATOM | 1558 | O1 | HOH | 449 | 0.421 | 18.268 | 48.931 | 1.00 | 46.66 |
| ATOM | 1559 | O1 | HOH | 450 | 2.933 | 26.992 | 56.334 | 1.00 | 74.71 |
| ATOM | 1560 | O1 | HOH | 451 | 0.759 | 26.113 | 64.001 | 1.00 | 59.06 |
| ATOM | 1561 | O1 | HOH | 452 | 1.084 | 6.151 | 31.450 | 1.00 | 56.68 |
| ATOM | 1562 | O1 | HOH | 453 | 5.884 | 4.519 | 29.654 | 1.00 | 56.39 |
| ATOM | 1563 | O1 | HOH | 454 | −15.425 | 18.155 | 64.235 | 1.00 | 72.96 |
| ATOM | 1564 | O1 | HOH | 455 | −0.171 | 13.254 | 39.710 | 1.00 | 74.60 |
| ATOM | 1565 | O1 | HOH | 456 | 14.866 | 20.258 | 26.412 | 1.00 | 46.29 |
| ATOM | 1566 | O1 | HOH | 457 | −14.275 | 3.131 | 87.758 | 1.00 | 59.31 |
| ATOM | 1567 | O1 | HOH | 458 | −4.332 | 5.479 | 80.377 | 1.00 | 79.39 |
| ATOM | 1568 | O1 | HOH | 459 | −2.873 | 25.788 | 71.842 | 1.00 | 79.58 |
| ATOM | 1569 | O1 | HOH | 460 | −18.438 | 26.604 | 40.069 | 1.00 | 61.56 |
| ATOM | 1570 | O1 | HOH | 461 | −17.600 | 14.799 | 67.139 | 1.00 | 57.68 |
| ATOM | 1571 | O1 | HOH | 462 | 18.682 | 10.751 | 18.572 | 1.00 | 52.48 |
| ATOM | 1572 | O1 | HOH | 463 | −1.155 | 16.670 | 9.382 | 1.00 | 52.73 |
| ATOM | 1573 | O1 | HOH | 464 | −7.994 | 4.789 | 73.142 | 1.00 | 58.99 |
| ATOM | 1574 | O1 | HOH | 465 | 2.878 | 15.527 | 89.468 | 1.00 | 53.82 |
| ATOM | 1575 | O1 | HOH | 466 | −14.384 | 15.627 | 85.915 | 1.00 | 59.37 |
| ATOM | 1576 | O1 | HOH | 467 | 2.898 | 22.812 | 45.093 | 1.00 | 59.10 |
| ATOM | 1577 | O1 | HOH | 468 | −0.751 | 11.409 | 92.643 | 1.00 | 70.59 |
| ATOM | 1578 | O1 | HOH | 469 | 5.961 | 11.789 | 58.672 | 1.00 | 63.17 |
| ATOM | 1579 | O1 | HOH | 470 | 17.813 | 12.012 | 26.528 | 1.00 | 46.16 |
| ATOM | 1580 | O1 | HOH | 471 | −12.149 | 25.188 | 31.130 | 1.00 | 55.19 |
| ATOM | 1581 | O1 | HOH | 472 | −15.854 | 29.561 | 37.766 | 1.00 | 78.50 |
| ATOM | 1582 | O1 | HOH | 473 | 0.237 | 23.917 | 15.562 | 1.00 | 69.78 |
| ATOM | 1583 | O1 | HOH | 474 | 1.289 | 4.512 | 6.743 | 1.00 | 81.85 |
| ATOM | 1584 | O1 | HOH | 476 | −8.588 | 25.220 | 33.408 | 1.00 | 70.20 |
| ATOM | 1585 | O1 | HOH | 477 | 6.612 | 5.903 | 2.424 | 1.00 | 69.70 |
| ATOM | 1586 | O1 | HOH | 478 | −11.595 | 11.694 | 52.934 | 1.00 | 67.26 |
| ATOM | 1587 | O1 | HOH | 479 | −6.127 | 9.039 | 42.656 | 1.00 | 74.38 |
| ATOM | 1588 | O1 | HOH | 480 | 4.050 | 28.730 | 37.477 | 1.00 | 74.96 |
| ATOM | 1589 | O1 | HOH | 481 | 8.643 | 22.280 | 26.068 | 1.00 | 66.23 |
| ATOM | 1590 | O1 | HOH | 482 | 5.057 | 18.842 | 63.792 | 1.00 | 84.51 |
| ATOM | 1591 | O1 | HOH | 483 | 11.232 | 20.655 | 13.298 | 1.00 | 78.39 |
| ATOM | 1592 | O1 | HOH | 484 | 8.429 | 7.903 | 29.673 | 1.00 | 63.19 |
| ATOM | 1593 | O1 | HOH | 485 | 2.108 | 19.893 | 73.346 | 1.00 | 55.11 |
| ATOM | 1594 | O1 | HOH | 486 | 6.372 | 25.790 | 68.263 | 1.00 | 65.80 |
| ATOM | 1595 | O1 | HOH | 487 | −13.205 | 18.613 | 68.124 | 1.00 | 86.13 |
| ATOM | 1596 | O1 | HOH | 488 | −8.286 | 11.850 | 17.825 | 1.00 | 45.69 |
| ATOM | 1597 | O1 | HOH | 489 | 7.059 | 5.906 | 5.738 | 1.00 | 58.55 |
| ATOM | 1598 | O1 | HOH | 490 | 2.959 | 27.083 | 27.258 | 1.00 | 52.49 |
| ATOM | 1599 | O1 | HOH | 491 | 2.930 | 19.627 | 39.466 | 1.00 | 56.92 |
| ATOM | 1600 | O1 | HOH | 492 | 13.904 | 13.945 | 10.116 | 1.00 | 53.45 |
| ATOM | 1601 | O1 | HOH | 493 | 6.999 | 6.052 | 26.735 | 1.00 | 66.18 |
| ATOM | 1602 | O1 | HOH | 494 | 18.877 | 11.256 | 9.403 | 1.00 | 62.85 |
| ATOM | 1603 | O1 | HOH | 495 | −14.299 | 15.165 | 36.983 | 1.00 | 54.65 |
| ATOM | 1604 | O1 | HOH | 496 | −2.145 | 20.024 | 90.307 | 1.00 | 58.77 |
| ATOM | 1605 | O1 | HOH | 497 | 12.960 | 14.184 | 7.677 | 1.00 | 44.90 |
| ATOM | 1606 | O1 | HOH | 498 | −0.577 | 22.082 | 22.522 | 1.00 | 84.17 |
| ATOM | 1607 | O1 | HOH | 499 | −8.915 | 23.163 | 54.891 | 1.00 | 47.87 |
| ATOM | 1608 | O1 | HOH | 500 | −5.904 | 19.990 | 16.699 | 1.00 | 68.72 |
| ATOM | 1609 | O1 | HOH | 501 | 7.604 | 22.425 | 14.079 | 1.00 | 83.85 |
| ATOM | 1610 | O1 | HOH | 502 | −5.745 | 23.331 | 22.836 | 1.00 | 71.67 |
| ATOM | 1611 | O1 | HOH | 503 | 12.713 | 6.777 | 5.150 | 1.00 | 68.39 |
| ATOM | 1612 | O1 | HOH | 504 | −8.006 | 21.972 | 84.763 | 1.00 | 67.50 |
| ATOM | 1613 | O1 | HOH | 505 | −6.453 | 17.333 | 12.456 | 1.00 | 75.63 |
| ATOM | 1614 | O1 | HOH | 506 | −3.191 | 6.789 | 49.810 | 1.00 | 63.94 |
| ATOM | 1615 | O1 | HOH | 507 | 4.446 | 24.626 | 63.212 | 1.00 | 85.62 |
| ATOM | 1616 | O1 | HOH | 508 | 10.825 | 10.927 | 25.007 | 1.00 | 52.28 |
| ATOM | 1617 | O1 | HOH | 509 | 1.751 | 28.971 | 44.260 | 1.00 | 88.85 |
| ATOM | 1618 | O1 | HOH | 510 | 5.304 | 11.175 | 65.843 | 1.00 | 86.55 |
| ATOM | 1619 | O1 | HOH | 511 | 3.016 | 25.072 | 34.499 | 1.00 | 56.55 |
| ATOM | 1620 | O1 | HOH | 512 | −7.777 | 21.602 | 36.643 | 1.00 | 88.35 |
| ATOM | 1621 | O1 | HOH | 513 | 11.033 | 20.553 | 20.646 | 1.00 | 42.22 |
| ATOM | 1622 | O1 | HOH | 514 | 1.225 | 4.220 | 72.811 | 1.00 | 68.80 |
| ATOM | 1623 | O1 | HOH | 600 | −6.830 | 12.211 | 91.855 | 1.00 | 60.53 |
| ATOM | 1625 | 01 | HOH | 602 | −6.789 | 6.449 | 80.935 | 1.00 | 50.26 |
| ATOM | 1626 | O1 | HOH | 603 | −2.982 | 21.809 | 7.218 | 1.00 | 69.39 |
| ATOM | 1627 | O1 | HOH | 604 | 13.293 | 8.016 | 2.296 | 1.00 | 61.13 |
| ATOM | 1628 | O1 | HOH | 605 | 3.299 | 11.650 | 51.045 | 1.00 | 78.04 |
| ATOM | 1629 | O1 | HOH | 606 | −16.971 | 17.233 | 50.226 | 1.00 | 68.89 |
| ATOM | 1630 | O1 | HOH | 607 | 2.846 | 12.738 | 39.593 | 1.00 | 71.60 |
| ATOM | 1631 | O1 | HOH | 608 | −3.147 | −2.544 | 9.531 | 1.00 | 58.55 |
| ATOM | 1632 | O1 | HOH | 609 | −4.861 | 3.305 | 80.691 | 1.00 | 60.83 |
| ATOM | 1633 | O1 | HOH | 610 | 6.834 | 13.354 | 71.357 | 1.00 | 64.45 |
| ATOM | 1634 | O1 | HOH | 611 | 2.963 | 4.487 | 27.971 | 1.00 | 48.70 |
| ATOM | 1635 | O1 | HOH | 612 | −2.162 | 14.623 | 70.194 | 1.00 | 72.85 |
| ATOM | 1636 | O1 | HOH | 613 | −8.983 | 11.246 | 91.235 | 1.00 | 68.16 |
| ATOM | 1637 | O1 | HOH | 614 | −1.414 | 15.727 | 89.255 | 1.00 | 63.90 |
| ATOM | 1638 | O1 | HOH | 615 | 1.353 | 25.378 | 70.806 | 1.00 | 68.29 |
| ATOM | 1639 | O1 | HOH | 616 | 0.315 | 14.016 | 78.504 | 1.00 | 59.91 |
| ATOM | 1640 | O1 | HOH | 617 | 4.477 | 9.852 | 70.092 | 1.00 | 79.40 |
| ATOM | 1641 | O1 | HOH | 618 | −12.259 | 11.420 | 46.776 | 1.00 | 58.20 |
| ATOM | 1642 | O1 | HOH | 619 | 2.679 | 4.422 | 32.337 | 1.00 | 49.91 |
| ATOM | 1643 | O1 | HOH | 620 | −17.285 | 12.212 | 71.786 | 1.00 | 68.80 |
| ATOM | 1644 | O1 | HOH | 621 | −17.043 | 8.933 | 85.930 | 1.00 | 71.94 |
| ATOM | 1645 | O1 | HOH | 622 | −12.833 | 21.451 | 57.231 | 1.00 | 69.93 |
| ATOM | 1646 | O1 | HOH | 623 | −16.934 | 18.199 | 72.267 | 1.00 | 64.58 |
| ATOM | 1647 | O1 | HOH | 624 | −1.857 | 26.990 | 67.917 | 1.00 | 52.07 |
| ATOM | 1648 | O1 | HOH | 625 | −13.989 | 23.289 | 43.768 | 1.00 | 68.81 |
| ATOM | 1649 | O1 | HOH | 626 | −8.279 | 10.487 | 46.008 | 1.00 | 64.05 |
| ATOM | 1650 | O1 | HOH | 627 | −8.410 | 14.523 | 46.407 | 1.00 | 94.70 |
| ATOM | 1651 | 01 | HOH | 628 | −14.238 | 29.716 | 34.051 | 1.00 | 74.01 |
| ATOM | 1652 | O1 | HOH | 629 | 2.497 | 10.302 | 52.957 | 1.00 | 78.36 |
| ATOM | 1653 | O1 | HOH | 630 | 12.781 | 18.085 | 14.371 | 1.00 | 59.38 |
| ATOM | 1654 | O1 | HOH | 631 | −13.379 | 8.527 | 74.408 | 1.00 | 72.72 |
| ATOM | 1655 | O1 | HOH | 632 | −8.436 | 24.292 | 72.901 | 1.00 | 80.80 |
| ATOM | 1656 | O1 | HOH | 633 | −11.927 | 12.370 | 43.399 | 1.00 | 72.78 |
| ATOM | 1657 | O1 | HOH | 634 | 3.105 | 21.424 | 61.107 | 1.00 | 80.29 |
| ATOM | 1658 | O1 | HOH | 635 | 1.552 | 18.331 | 85.585 | 1.00 | 75.99 |
| ATOM | 1659 | O1 | HOH | 636 | −13.619 | 20.355 | 61.490 | 1.00 | 63.28 |
| ATOM | 1660 | O1 | HOH | 637 | −2.200 | 8.533 | 10.367 | 1.00 | 63.23 |
| ATOM | 1661 | O1 | HOH | 638 | 3.589 | 19.637 | 44.461 | 1.00 | 47.03 |
| ATOM | 1662 | O1 | HOH | 639 | −1.573 | 21.818 | 9.438 | 1.00 | 68.93 |
| ATOM | 1663 | O1 | HOH | 640 | 6.258 | 22.557 | 73.322 | 1.00 | 58.13 |
| ATOM | 1664 | O1 | HOH | 641 | −13.066 | 10.161 | 44.534 | 1.00 | 63.83 |
| ATOM | 1665 | O1 | HOH | 642 | −9.747 | 28.802 | 92.952 | 1.00 | 80.11 |
| ATOM | 1666 | O1 | HOH | 643 | −5.127 | 4.127 | 4.706 | 1.00 | 79.47 |
| ATOM | 1667 | O1 | HOH | 644 | −13.918 | 12.183 | 81.003 | 1.00 | 58.64 |
| ATOM | 1668 | O1 | HOH | 645 | 19.938 | 9.499 | 13.944 | 1.00 | 87.18 |
| ATOM | 1669 | HG | MER | 701 | 9.336 | 0.982 | 15.636 | 0.20 | 58.21 |
| ATOM | 1670 | HG | MER | 702 | 9.841 | 5.199 | 14.508 | 0.20 | 67.41 |
| ATOM | 1671 | HG | MER | 703 | −7.127 | 10.772 | 57.593 | 0.20 | 79.35 |
| ATOM | 1672 | C1 | DMA | 801 | −13.170 | 18.614 | 25.453 | 1.00 | 77.87 |
| ATOM | 1674 | C1′ | DMA | 801 | −11.027 | 17.896 | 26.233 | 1.00 | 77.70 |
| ATOM | 1675 | C2′ | DMA | 801 | −9.669 | 18.447 | 26.518 | 1.00 | 75.99 |
| ATOM | 1676 | C3′ | DMA | 801 | −8.688 | 17.714 | 25.612 | 1.00 | 72.89 |
| ATOM | 1677 | C4′ | DMA | 801 | −7.247 | 18.219 | 25.835 | 1.00 | 69.58 |
| ATOM | 1678 | C5′ | DMA | 801 | −6.263 | 17.478 | 24.914 | 1.00 | 65.32 |
| ATOM | 1679 | C6′ | DMA | 801 | −6.218 | 16.101 | 25.451 | 1.00 | 61.15 |
| ATOM | 1680 | C7′ | DMA | 801 | −5.365 | 15.188 | 24.661 | 1.00 | 59.83 |
| ATOM | 1681 | C8′ | DMA | 801 | −5.575 | 13.886 | 25.325 | 1.00 | 64.73 |
| ATOM | 1682 | C9′ | DMA | 801 | −4.915 | 12.693 | 24.717 | 1.00 | 64.11 |
| ATOM | 1683 | C10′ | DMA | 801 | −5.345 | 11.526 | 25.553 | 1.00 | 67.12 |
| ATOM | 1684 | O1 | DMA | 801 | −11.982 | 18.934 | 26.182 | 1.00 | 79.61 |
| ATOM | 1685 | O5 | DMA | 801 | −12.859 | 18.362 | 24.066 | 1.00 | 75.75 |

The following abbreviations are used in Table 2:
“Atom Type” refers to the element whose coordinates are provided. The first letter in the column defines the element. For example, that beginning with “O” indicates oxygen, “C” carbon, “N” nitrogen, and “S” sulfur. But “HG” Indicates mercury.
“A.A.” refers to amino acid. In this column, HOH indicates water molecule, MER does mercury atom, and DMA does the atom of detergent.
In the column of “A.A. No.”, 1-185 indicate 185 of amino acids in RRF, 302-645 do water molecules, 701-703 do mercury atoms, and 801 does the detergent.
“X, Y and Z” provide the Cartesian coordinates of the element.
“B” is a thermal factor that measures movement of the atom around its atom center.
“OCC” refers to occupancy and represents the degree that the atom occupies the particular coordinate. For example, 1.0 indicates that one atom is located on the coordinate. 0.2 indicates that 0.2 of atom is located.

While the present invention has been described with respect to certain preferred embodiments, other modifications and variations may be made without departing from the spirit and scope of the present invention as set forth in the following claims.

What is claimed is:

1. A crystal of ribosome recycling factor (RRF) or an RRF homolog comprising:
RRF or an RRF mutant or an RRF homolog, which has substantially the same structure as that of RRF and whose amino acid sequence is formed by substituting, or eliminating at least one amino acid residue in the RRF amino acid sequence or by adding at least one amino acid residue to the RRF sequence; and
a compound, which can bind to the hydrophobic cleft formed between two domains of RRF.

2. The crystal of RRF or an RRF homolog according to claim 1, wherein the compound is a compound having 3 to 12 carbon atoms on a straight chain alkyl group, or a compound having 3 to 12 carbon atoms on a straight chain alkyl group and a functional group which can form a non-hydrophobic bond.

3. The crystal of RRF or an RRF homolog according to claim 1, wherein the compound is decyl-□-D-maltopyranoside.

4. The crystal of RRF or an RRF homolog according to claim 1, wherein the RRF or RRF homolog has the three-dimensional structure specified by 0.2 nm of root mean square deviation (rmsd) between the coordinate of each atom on the RRF or RRF homolog and the corresponding coordinate of each atom described in table 2.

5. A crystallization method for RRF or an RRF homolog comprising the step of crystallizing RRF, an RRF mutant or an RRF homolog with a compound binding to the hydrophobic cleft formed between two domains of RRF, wherein the RRF mutant or RRF homolog has substantially the same structure as that of RRF and wherein the amino acid sequence of the RRF mutant or homolog is formed by substituting, or eliminating at least one amino acid residue in the RRF amino acid sequence or by adding at least one amino acid residue to the RRF sequence.

6. The crystallization method according to claim 5, wherein said compound comprises 3-12 carbon atoms on a straight chain alkyl group, or 3-12 carbon atoms on a straight chain alkyl group and a functional group which can form a non-hydrophobic bond.

7. The crystallization method according to claim 5, wherein said compound is decyl-β-D-maltopyranoside.

8. An inhibitor of RRF comprising the coiled coil domain of RRF or an RRF homolog wherein said coiled coil domain binds to the hydrophobic cleft of the RRF or RRF homolog.

9. A method for identifying an inhibitor of RRF comprising the steps of:
selecting or designing an inhibitor candidate compound or its fragment from a database using the three-dimensional structure of RRF defined by the coordinates of atoms in RRF described in table 2;
inserting the compound or fragment into the three-dimensional structure of RRF using a computer modeling method;
determining the fit of the inhibitor candidate with RRF; and
selecting as an inhibitor the compound or fragment which fits in such a way that it will inhibit the activity of RRF.

10. The method according to claim 9 further comprising the steps of modifying the compound or fragment using the three-dimensional structure of RRF and repeating the steps of inserting, determining and selecting as an inhibitor.

11. The method according to claim 9 further comprising the steps of designing a compound by linking two or more of the compounds or fragments wherein said compounds or fragments bind to different parts of RRF, and repeating the steps of inserting, determining and selecting as an inhibitor.

12. The method according to claim 9 further comprising the steps of:
preparing the inhibitor of RRF;
identifying whether the compound inhibits the activity of RRF in a prokaryote in vivo or in vitro; and
modifying the inhibitor of RRF using the three-dimensional structure of RRF, and repeating the steps of inserting, determining and selecting as an inhibitor.

13. The method according to claim 9, wherein the three-dimensional structure of RRF used in the analysis is the three-dimensional structure formed by the hydrophobic cleft and its peripheral or circumferential amino acid residues.

14. The method according to claim 13, wherein the inhibitor comprises an alkyl group which binds to the hydrophobic cleft of RRF and further comprising a functional group non-hydrophobically binding to the peripheral or circumferential residues near the hydrophobic cleft.

15. The method according to claim 9, wherein the step of determining the fit of the inhibitor with RRF comprises: determining the fit of the inhibitor with RRF when the dissociation constant of the interaction is 100 micromoles or less.

16. The method according to claim 9, wherein the three dimensional structure of RRF used for the analysis is that of the coiled coil domain of RRF.

17. The method according to claim 16 further comprising the steps of:
preparing an RRF mutant, in which at least one residue is substituted on the surface of the coiled coil domain of RRF;
preparing a prokaryote which expresses the RRF mutant; and
identifying whether the RRF mutant reduces the growth of said prokaryote
designing a compound comprising at least the substituted residue in the RRF;
preparing the compound;
identifying the inhibition activity of the compound.

18. An isolated polypeptide comprising substantially the same three-dimensional structure as that of the coiled coil domain of RRF.

19. The isolated polypeptide of claim 18 comprising sequences selected from the group consisting of SEQ ID NOS:1-4 or truncated versions thereof.

20. A method for identifying an inhibitor of RRF comprising the steps of:
preparing a mutant, in which at least one residue is substituted on the surface of the coiled coil domain of RRF;
checking the growth of a prokaryote expressing the mutant RRF;
identifying those mutants of RRF that cause said prokaryotes to have a reduced growth rate as containing mutation at residues involved in binding of RRF to the ribosome.

21. The method according to claim 20, wherein when the residues in the binding site or part are isolated by other residue(s) in the domain, the compound is designed by linking the residues in the binding site or part with linker in the designing step.
